(19) United States
(12) Patent Application Publication
(10) Pub. No.: US 2019/0216943 A1
(43) Pub. Date: Jul. 18, 2019

(54) TGF-BETA ANTAGONIST CONJUGATES

(71) Applicant: PreciThera, Inc., Montreal (CA)

(72) Inventors: Philippe CRINE, Montreal (CA); Susan SCHIAVI, Hopkinton, MA (US)

(21) Appl. No.: 16/324,501

(22) PCT Filed: Aug. 11, 2017
(86) PCT No.: PCT/CA2017/050956

§ 371 Date: Feb. 8, 2019

Related U.S. Application Data
(60) Provisional application No. 62/373,597, filed on Aug. 11, 2016.

(51) Int. Cl. A61K 47/68; A61K 9/00; A61P 19/08; A61K 31/4439; A61K 47/64

(52) U.S. Cl. CPC A61K 47/6845; A61K 9/0019; A61P 19/08; A61K 47/6803; A61K 47/645; A61K 47/6811; A61K 31/4439

(57) ABSTRACT

The present invention provides conjugates containing a (i) TGF-β antagonist, such as a TGF-β antagonist antibody, protein, peptide, or small molecule capable of inhibiting TGF-β signaling, bound to (ii) a bone-targeting moiety. The bone targeting moiety localizes the TGF-β antagonist to osseous tissue. 0 The conjugates described herein provide a therapeutic paradigm for the treatment of various diseases associated with elevated TGF-β signaling and elevated bone turnover, such as osteogenesis imperfecta and other bone pathologies.

### FIELD OF THE INVENTION

The present invention relates to the fields of peptide and protein therapy and provides therapeutic conjugates capable of localizing to osseous tissue and attenuating TGF-β signaling for the treatment of pathologies associated with elevated TGF-β signaling and bone turnover.

### BACKGROUND OF THE INVENTION

Transforming growth factor-β (TGF-β) is an important regulator of bone homeostasis, and the activity of this protein promotes a balance between bone building and degradation. Elevations in active TGF-β and downstream signaling are associated with a variety of pathologies. There remains a need for the development of therapeutic compounds capable of attenuating TGF-β signal transduction at the site of bone tissue.

### SUMMARY OF THE INVENTION

The invention provides therapeutic conjugates containing TGF-β antagonists, such as TGF-β antagonist peptides, bound to a targeting moiety that localizes to human bone tissue. These constructs can be used to treat a variety of pathologies, such as those associated with elevated TGF-β signaling and/or bone turnover. A wide array of TGF-β antagonists, such as TGF-β antagonist proteins, peptides, and antibodies, can be used in conjunction with the compositions and methods described herein. For instance, therapeutic conjugates of the invention can include TGF-β antagonists, such as antagonistic proteins, peptides, and antibodies, that bind and inhibit TGF-β. In some embodiments, the therapeutic conjugate contains a TGF-β antagonist, such as a protein, peptide, or antibody, that binds and inhibits a TGF-β receptor. Similarly, a variety of bone targeting moieties can be incorporated into the therapeutic conjugates described herein. Exemplary bone-targeting moieties include those that specifically bind proteins and minerals present in human bone tissue, such as collagen and hydroxyapatite, respectively. The therapeutic conjugates described herein can be administered to a patient (e.g., a mammalian patient, such as a human patient) for the treatment of a variety of pathological conditions in which TGF-β signaling is aberrantly regulated, such as conditions in which bone turnover is elevated relative to a healthy subject.

In a first aspect, the invention provides a conjugate containing a TGF-β antagonist bound to a targeting moiety. The targeting moiety can bind, for example, a protein, such as collagen, or mineral, such as hydroxyapatite, present in human bone tissue.

In some embodiments, the TGF-β antagonist binds TGF-β. In some embodiments, the TGF-β antagonist binds and neutralizes TGF-β, for instance, thereby suppressing TGF-β signal transduction. In some embodiments, the TGF-β antagonist comprises a protein, peptide, antibody, or small molecule, such as a protein, peptide, antibody, or small molecule that binds TGF-β. For instance, in some embodiments, the TGF-β antagonist is a protein, peptide, antibody, or small molecule, such as a protein, peptide, antibody, or small molecule that binds and inhibits the activity of TGF-β or a TGF-β receptor.

In some embodiments, the TGF-β antagonist is a protein, peptide, or antibody, such as a protein, peptide, or antibody that binds and inhibits the activity of TGF-β or a TGF-β receptor.

In some embodiments, the TGF-β antagonist is a peptide or antibody, such as a peptide or antibody that binds and inhibits the activity of TGF-β or a TGF-β receptor.

In some embodiments, the TGF-β antagonist is a small molecule, such as a small molecule that binds and inhibits the activity of TGF-β or a TGF-β receptor.

In some embodiments, the TGF-β antagonist is a peptide. In some embodiments, the peptide contains the amino acid sequence IDGVYDNAEYAERFMEENEGHIVDIHDFSLGSS (SEQ ID NO: 5), or a sequence having at least 85% sequence identity (e.g., at least 85%, 90%, 95%, 97%, 99%, or greater) thereto and/or having one or more conservative amino acid substitutions with respect to this sequence.

In some embodiments, the peptide contains the amino acid sequence WIWLDTNMGYRIYQEFEVT (SEQ ID NO: 1), or a sequence having at least 85% sequence identity (e.g., at least 85%, 90%, 95%, 97%, 99%, or greater) thereto and/or having one or more conservative amino acid substitutions with respect to this sequence. In some embodiments, the peptide contains the amino acid sequence of residues 21-1404 of SEQ ID NO: 2, or a sequence having at least 85% sequence identity (e.g., at least 85%, 90%, 95%, 97%, 99%, or greater) thereto and/or having one or more conservative amino acid substitutions with respect to this sequence. In some embodiments, the peptide contains the amino acid sequence of residues 21-1428 of SEQ ID NO: 2, or a sequence having at least 85% sequence identity (e.g., at least 85%, 90%, 95%, 97%, 99%, or greater) thereto and/or having one or more conservative amino acid substitutions with respect to this sequence. In some embodiments, the peptide contains the amino acid sequence of SEQ ID NO: 2, or a sequence having at least 85% sequence identity (e.g., at least 85%, 90%, 95%, 97%, 99%, or greater) thereto and/or having one or more conservative amino acid substitutions with respect to this sequence.

In some embodiments, the peptide contains the amino acid sequence WIWLDTNMGSRIYQEFEVT (SEQ ID NO: 3), or a sequence having at least 85% sequence identity (e.g., at least 85%, 90%, 95%, 97%, 99%, or greater) thereto and/or having one or more conservative amino acid substitutions with respect to this sequence. In some embodiments, the peptide contains the amino acid sequence of residues 21-1404 of SEQ ID NO: 4, or a sequence having at least 85% sequence identity (e.g., at least 85%, 90%, 95%, 97%, 99%, or greater) thereto and/or having one or more conservative amino acid substitutions with respect to this sequence. In some embodiments, the peptide contains the amino acid sequence of residues 21-1428 of SEQ ID NO: 4, or a sequence having at least 85% sequence identity (e.g., at least 85%, 90%, 95%, 97%, 99%, or greater) thereto and/or having one or more conservative amino acid substitutions with respect to this sequence. In some embodiments, the peptide contains the amino acid sequence of SEQ ID NO: 4, or a sequence having at least 85% sequence identity (e.g., at least 85%, 90%, 95%, 97%, 99%, or greater) thereto and/or having one or more conservative amino acid substitutions with respect to this sequence.

In some embodiments, the peptide contains the amino acid sequence of SEQ ID NO: 6, or a sequence having at least 85% sequence identity (e.g., at least 85%, 90%, 95%, 97%, 99%, or greater) thereto and/or having one or more conservative amino acid substitutions with respect to this sequence.

In some embodiments, the peptide contains the amino acid sequence of RKHFPETWIWLDTNMGYRIYQEFEV (SEQ ID NO: 7), or a sequence having at least 85% sequence identity (e.g., at least 85%, 90%, 95%, 97%, 99%, or greater) thereto and/or having one or more conservative amino acid substitutions with respect to this sequence.

In some embodiments, the peptide contains an amino acid sequence selected from the group consisting of ANFCLGPCPYIWSLDT (SEQ ID NO: 8), ANFCSGPCPYLRSADT (SEQ ID NO: 9), PYIWSLDTQY (SEQ ID NO: 10), PYLWSSDTQH (SEQ ID NO: 11), PYLRSADTTH (SEQ ID NO: 12), WSXD (SEQ ID NO: 13), and RSXD (SEQ ID NO: 14), wherein X represents any naturally occurring amino acid. In some embodiments, the peptide contains an amino acid sequence having at least 85% sequence identity (e.g., at least 85%, 90%, 95%, 97%, 99%, or greater) to one of these sequences and/or having one or more conservative amino acid substitutions with respect to one of these sequences.

In some embodiments, the peptide contains an amino acid sequence selected from the group consisting of TSLDATMIWTMM (SEQ ID NO: 15), SNPYSAFQVDIIVDI (SEQ ID NO: 16), TSLMIWTMM (SEQ ID NO: 17), TSLDASIIWAMMQN (SEQ ID NO: 18), SNPYSAFQVDITID (SEQ ID NO: 19), EAVLILQGPPYVSWL (SEQ ID NO: 20), and LDSLSFQLGLYLSPH (SEQ ID NO: 21). In some embodiments, the peptide contains an amino acid sequence having at least 85% sequence identity (e.g., at least 85%, 90%, 95%, 97%, 99%, or greater) to one of these sequences and/or having one or more conservative amino acid substitutions with respect to one of these sequences.

In some embodiments, the peptide contains an amino acid sequence selected from the group consisting of TSLDASIIWAMMQN (SEQ ID NO: 22), KRIWFIPRSSWYERA (SEQ ID NO: 23), KRIWFIPRSSW (SEQ ID NO: 24), KRIWFIPRSSW (SEQ ID NO: 25), and KRIWFIPRSSW (SEQ ID NO: 26). In some embodiments, the peptide contains an amino acid sequence having at least 85% sequence identity (e.g., at least 85%, 90%, 95%, 97%, 99%, or greater) to one of these sequences and/or having one or more conservative amino acid substitutions with respect to one of these sequences.

In some embodiments, the peptide contains the amino acid sequence of any one of SEQ ID NOs: 27-49. In some embodiments, the peptide contains an amino acid sequence having at least 85% sequence identity (e.g., at least 85%, 90%, 95%, 97%, 99%, or greater) to one of these sequences and/or having one or more conservative amino acid substitutions with respect to one of these sequences.

In some embodiments, the peptide contains the amino acid sequence of glycoprotein-A repetitions predominant protein (GARP) (SEQ ID NO: 50). In some embodiments, the peptide contains an amino acid sequence having at least 85% sequence identity (e.g., at least 85%, 90%, 95%, 97%, 99%, or greater) to this sequences and/or having one or more conservative amino acid substitutions with respect to this sequence.

In some embodiments, the TGF-β antagonist contains a peptide that binds a TGF-β receptor. In some embodiments, the TGF-β antagonist is a peptide that binds a TGF-β receptor. For instance, in some embodiments, the peptide contains an amino acid sequence selected from the group consisting of HANFCLGPCPYIWSL (SEQ ID NO: 51), FCLGPCPYIWSLDT (SEQ ID NO: 52), and HEPKGYHANFCLGPCP (SEQ ID NO: 53). In some embodiments, the peptide contains an amino acid sequence having at least 85% sequence identity (e.g., at least 85%, 90%, 95%, 97%, 99%, or greater) to one of these sequences and/or having one or more conservative amino acid substitutions with respect to one of these sequences.

In some embodiments, the TGF-β antagonist is an antibody or an antigen-binding fragment thereof that binds TGF-β, such as an isoform of TGF-β (e.g., TGF-β1, TGF-β2, and/or TGF-β3). In some embodiments, the antibody or antigen-binding fragment thereof contains one or more, or all, of the following complementarity determining regions (CDRs):
a. a CDR-H1 having the amino acid sequence SNVIS (SEQ ID NO: 327);
b. a CDR-H2 having the amino acid sequence GVIPIVDIANYAQRFKG (SEQ ID NO: 328);
c. a CDR-H3 having the amino acid sequence TLGLVLDAMDY (SEQ ID NO: 329);
d. a CDR-L1 having the amino acid sequence RASQSLGSSYLA (SEQ ID NO: 330);
e. a CDR-L2 having the amino acid sequence GASSRAP (SEQ ID NO: 331); and
f. a CDR-L3 having the amino acid sequence QQYADSPIT (SEQ ID NO: 332).

In some embodiments, the antibody or antigen-binding fragment thereof competitively inhibits the binding of TGF-β to an antibody or antigen binding fragment thereof that contains the following complementarity determining regions (CDRs):
a. a CDR-H1 having the amino acid sequence SNVIS (SEQ ID NO: 327);
b. a CDR-H2 having the amino acid sequence GVIPIVDIANYAQRFKG (SEQ ID NO: 328);
c. a CDR-H3 having the amino acid sequence TLGLVLDAMDY (SEQ ID NO: 329);
d. a CDR-L1 having the amino acid sequence RASQSLGSSYLA (SEQ ID NO: 330);
e. a CDR-L2 having the amino acid sequence GASSRAP (SEQ ID NO: 331); and
f. a CDR-L3 having the amino acid sequence QQYADSPIT (SEQ ID NO: 332).

In some embodiments, the antibody or antigen-binding fragment thereof contains one or more CDRs that have at least 85% sequence identity (e.g., at least 85%, 90%, 95%, 97%, 98%, 99%, or more, sequence identity) to the corresponding CDRs of SEQ ID NOs: 327-332. In some embodiments, the antibody or antigen-binding fragment thereof contains a set of six CDRs that each have at least 85% sequence identity (e.g., at least 85%, 90%, 95%, 97%, 98%, 99%, or more, sequence identity) to the foregoing CDRs.

In some embodiments, the antibody contains a heavy chain variable region having the amino acid sequence of QVQLVQSGAEVKKPGSSVKVSCKASGYTFSSNVISWVRQAPGQGLEWMGGVIPIVDIANY AQRFKGRVTITADESTSTTYMELSSLRSEDTAVYYCASTLGLVLDAMDYWGQGTLVTVSS (SEQ ID NO: 333), or a heavy chain variable region having an amino acid sequence that has at least 85% sequence identity (e.g., at least 85%, 90%, 95%, 97%, 98%, 99%, or more, sequence identity) to SEQ ID NO: 333. In some embodiments, the antibody or antigen-binding fragment thereof has a light chain variable region having the amino acid sequence of ETVLTQSPGTLSLSPGERATLSCRASQSLGSSYLAWYQQKPGQAPRLLIYGASSRAPGIP DRFSGSGSGTDFTLTISRLEPEDFAVYYCQQYADSPITFGQGTRLEIK (SEQ ID NO: 334), or a light chain variable region having an amino acid sequence that has at least 85% sequence identity (e.g., at least 85%, 90%, 95%, 97%, 98%, 99%, or more, sequence identity) to SEQ ID NO: 334. Antibodies containing the foregoing CDRs, as well as the above heavy chain variable region and light chain variable regions, are described, e.g., in U.S. Pat. No. 9,598,486, the disclosure of which is incorporated herein by reference in its entirety.

In some embodiments, the antibody or antigen-binding fragment thereof is a monoclonal antibody or antigen-binding fragment thereof, a polyclonal antibody or antigen-binding fragment thereof, a humanized antibody or antigen-binding fragment thereof, a bispecific antibody or antigen-binding fragment thereof, an optimized antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, or a tandem di-scFV.

In some embodiments, the antibody or antigen-binding fragment thereof is a humanized antibody or antigen-binding fragment thereof, such as a humanized antibody or antigen-binding fragment thereof of the 1D11 antibody, described herein.

In some embodiments, the antibody or antigen-binding fragment thereof is an optimized antibody or antigen-binding fragment thereof, such as an optimized variant of the 1D11 and/or GC1008 antibodies, described herein.

In some embodiments, the optimized antibody or antigen-binding fragment thereof is an affinity-matured antibody or antigen-binding fragment thereof, such as an affinity-matured variant of the 1D11 and/or GC1008 antibodies, described herein.

In some embodiments, the antibody is a single-chain molecule, such as a scFv, a diabody, or a triabody, among others described herein.

In some embodiments, the antibody is a scFv.

In some embodiments, the targeting moiety contains a peptide, such as a peptide that binds a protein present in human bone tissue. In some embodiments, the targeting moiety is a peptide, such as a peptide that binds a protein present in human bone tissue. In some embodiments, the protein present in human bone tissue is collagen. For instance, the peptide that binds the protein may contain the amino acid sequence of any one of SEQ ID NOs: 54-56. In some embodiments, the peptide that binds the protein contains the amino acid sequence of any one of SEQ ID NOs: 57-59. In some embodiments, the peptide that binds the protein contains the amino acid sequence of SEQ ID NO: 56. In some embodiments, the peptide that binds the protein contains an amino acid sequence having at least 85% sequence identity (e.g., at least 85%, 90%, 95%, 97%, 99%, or greater) to one of these sequences and/or having one or more conservative amino acid substitutions with respect to one of these sequences.

In some embodiments, the targeting moiety contains a peptide capable of binding a mineral present in human bone tissue, such as hydroxyapatite. In some embodiments, the peptide that binds the mineral contains the amino acid sequence of any one of SEQ ID NOs: 60-326. In some embodiments, the peptide that binds the mineral contains an amino acid sequence having at least 85% sequence identity (e.g., at least 85%, 90%, 95%, 97%, 99%, or greater) to one of these sequences and/or having one or more conservative amino acid substitutions with respect to one of these sequences.

In some embodiments, the targeting moiety capable of binding hydroxyapatite is a polyanionic peptide. The polyanionic peptide may contain, for instance, one or more amino acids bearing a side-chain substituent selected from the group consisting of carboxylate, sulfonate, phosphonate, and phosphate.

In some embodiments, the polyanionic peptide contains (e.g., consists of) one or more glutamate residues (e.g., 1-25 glutamate residues, or more, such as 1, 2, 3, 4, 5, 6, 7, 8, 9, 10, 11, 12, 13, 14, 15, 16, 17, 18, 19, 20, 21, 22, 23, 24, or 25, or more, glutamate residues). In some embodiments, the polyanionic peptide contains (e.g., consists of) from 3 to 20 glutamate residues (e.g., 3, 4, 5, 6, 7, 8, 9, 10, 11, 12, 13, 14, 15, 16, 17, 18, 19, or 20 glutamate residues). In some embodiments, the polyanionic peptide contains (e.g., consists of) from 5 to 15 glutamate residues (e.g., 5, 6, 7, 8, 9, 10, 11, 12, 13, 14, or 15 glutamate residues). In some embodiments, the polyanionic peptide contains (e.g., consists of) from 8 to 12 glutamate residues (e.g., 8, 9, 10, 11, or 12 glutamate residues). In some embodiments, the polyanionic peptide contains (e.g., consists of) 5 glutamate residues. In some embodiments, the polyanionic peptide contains (e.g., consists of) 6 glutamate residues. In some embodiments, the polyanionic peptide contains (e.g., consists of) 7 glutamate residues. In some embodiments, the polyanionic peptide contains (e.g., consists of) 8 glutamate residues. In some embodiments, the polyanionic peptide contains (e.g., consists of) 9 glutamate residues. In some embodiments, the polyanionic peptide contains (e.g., consists of) 10 glutamate residues. In some embodiments, the polyanionic peptide contains (e.g., consists of) 11 glutamate residues. In some embodiments, the polyanionic peptide contains (e.g., consists of) 12 glutamate residues. In some embodiments, the polyanionic peptide contains (e.g., consists of) 13 glutamate residues. In some embodiments, the polyanionic peptide contains (e.g., consists of) 14 glutamate residues. In some embodiments, the polyanionic peptide contains (e.g., consists of) 15 glutamate residues.

In some embodiments, the polyanionic peptide is a peptide of the formula Eₙ, wherein E designates a glutamate residue and n is an integer from 1 to 25. For instance, the polyanionic peptide may be of the formula E₁, E₂, F₃, E₄, F₅, E₆, E₇, E₈, E₉, E₁₀, E₁₁, E₁₂, E₁₃, E₁₄, E₁₅, E₁₆, E₁₇, E₁₈, E₁₉, E₂₀, E₂₁, E₂₂, E₂₃, E₂₄, or E₂₅. In some embodiments, the peptide is a peptide of the formula XₙEₘXₒEₚ, wherein E designates a glutamate residue, each X independently designates any naturally-occurring amino acid, m represents an integer from 1 to 25, and n and o each independently represent integers from 0 to 5, and p represents an integer from 1 to 10.

For instance, in some embodiments, the polyanionic peptide is a peptide of the formula E₂. In some embodiments, the polyanionic peptide is a peptide of the formula E₃. In some embodiments, the polyanionic peptide is a peptide of the formula E_{q}. In some embodiments, the polyanionic peptide is a peptide of the formula E₅. In some embodiments, the polyanionic peptide is a peptide of the formula E₆. In some embodiments, the polyanionic peptide is a peptide of the formula E₇. In some embodiments, the polyanionic peptide is a peptide of the formula E₈. In some embodiments, the polyanionic peptide is a peptide of the formula E₉. In some embodiments, the polyanionic peptide is a peptide of the formula E₁₀. In some embodiments, the polyanionic peptide is a peptide of the formula E₁₁. In some embodiments, the polyanionic peptide is a peptide of the formula E₁₂. In some embodiments, the polyanionic peptide is a peptide of the formula E₁₃. In some embodiments, the polyanionic peptide is a peptide of the formula E₁₄. In some embodiments, the polyanionic peptide is a peptide of the formula E₁₅. In some embodiments, the polyanionic peptide is a peptide of the formula E₁₆. In some embodiments, the polyanionic peptide is a peptide of the formula E₁₇. In some embodiments, the polyanionic peptide is a peptide of the formula E₁₈. In some embodiments, the polyanionic peptide is a peptide of the formula E₁₉. In some embodiments, the polyanionic peptide is a peptide of the formula E₂₀. In some embodiments, the polyanionic peptide is a peptide of the formula E₂₁. In some embodiments, the polyanionic peptide is a peptide of the formula E₂₂. In some embodiments, the polyanionic peptide is a peptide of the formula E₂₃. In some embodiments, the polyanionic peptide is a peptide of the formula E₂₄. In some embodiments, the polyanionic peptide is a peptide of the formula E₂₅.

In some embodiments, the polyanionic peptide is a peptide of the formula E₁₀.

In some embodiments, the glutamate residues are consecutive. In some embodiments, the glutamate residues are discontinuous.

In some embodiments, the polyanionic peptide contains (e.g., consists of) one or more aspartate residues (e.g., 1-25 aspartate residues, or more, such as 1, 2, 3, 4, 5, 6, 7, 8, 9, 10, 11, 12, 13, 14, 15, 16, 17, 18, 19, 20, 21, 22, 23, 24, or 25, or more, aspartate residues). In some embodiments, the polyanionic peptide contains (e.g., consists of) from 3 to 20 aspartate residues (e.g., 3, 4, 5, 6, 7, 8, 9, 10, 11, 12, 13, 14, 15, 16, 17, 18, 19, or 20 aspartate residues). In some embodiments, the polyanionic peptide contains (e.g., consists of) from 5 to 15 aspartate residues (e.g., 5, 6, 7, 8, 9, 10, 11, 12, 13, 14, or 15 aspartate residues). In some embodiments, the polyanionic peptide contains (e.g., consists of) from 8 to 12 aspartate residues (e.g., 8, 9, 10, 11, or 12 aspartate residues). In some embodiments, the polyanionic peptide contains (e.g., consists of) 5 aspartate residues. In some embodiments, the polyanionic peptide contains (e.g., consists of) 6 aspartate residues. In some embodiments, the polyanionic peptide contains (e.g., consists of) 7 aspartate residues. In some embodiments, the polyanionic peptide contains (e.g., consists of) 8 aspartate residues. In some embodiments, the polyanionic peptide contains (e.g., consists of) 9 aspartate residues. In some embodiments, the polyanionic peptide contains (e.g., consists of) 10 aspartate residues. In some embodiments, the polyanionic peptide contains (e.g., consists of) 11 aspartate residues. In some embodiments, the polyanionic peptide contains (e.g., consists of) 12 aspartate residues. In some embodiments, the polyanionic peptide contains (e.g., consists of) 13 aspartate residues. In some embodiments, the polyanionic peptide contains (e.g., consists of) 14 aspartate residues. In some embodiments, the polyanionic peptide contains (e.g., consists of) 15 aspartate residues.

In some embodiments, the polyanionic peptide is a peptide of the formula Dₙ, wherein D designates an aspartate residue and n is an integer from 1 to 25. For instance, the polyanionic peptide may be of the formula D₁, D₂, D₃, D₄, D₅, D₆, D₇, D₈, D₉, D₁₀, D₁₁, D₁₂, D₁₃, D₁₄, D₁₅, D₁₆, D₁₇, D₁₈, D₁₉, D₂₀, D₂₁, D₂₂, D₂₃, D₂₄, or D₂₅. In some embodiments, the peptide is a peptide of the formula XₙDₘXₒDₚ, wherein D designates an aspartate residue, each X independently designates any naturally-occurring amino acid, m represents an integer from 1 to 25, and n and o each independently represent integers from 0 to 5, and p represents an integer from 1 to 10.

For instance, in some embodiments, the polyanionic peptide is a peptide of the formula D₂. In some embodiments, the polyanionic peptide is a peptide of the formula D₃. In some embodiments, the polyanionic peptide is a peptide of the formula D₄. In some embodiments, the polyanionic peptide is a peptide of the formula D₅. In some embodiments, the polyanionic peptide is a peptide of the formula D₆. In some embodiments, the polyanionic peptide is a peptide of the formula D₇. In some embodiments, the polyanionic peptide is a peptide of the formula D₈. In some embodiments, the polyanionic peptide is a peptide of the formula D₉. In some embodiments, the polyanionic peptide is a peptide of the formula D₁₀. In some embodiments, the polyanionic peptide is a peptide of the formula D₁₁. In some embodiments, the polyanionic peptide is a peptide of the formula D₁₂. In some embodiments, the polyanionic peptide is a peptide of the formula D₁₃. In some embodiments, the polyanionic peptide is a peptide of the formula D₁₄. In some embodiments, the polyanionic peptide is a peptide of the formula D₁₅. In some embodiments, the polyanionic peptide is a peptide of the formula D₁₆. In some embodiments, the polyanionic peptide is a peptide of the formula D₁₇. In some embodiments, the polyanionic peptide is a peptide of the formula D₁₈. In some embodiments, the polyanionic peptide is a peptide of the formula D₁₉. In some embodiments, the polyanionic peptide is a peptide of the formula D₂₀. In some embodiments, the polyanionic peptide is a peptide of the formula D₂₁. In some embodiments, the polyanionic peptide is a peptide of the formula D₂₂. In some embodiments, the polyanionic peptide is a peptide of the formula D₂₃. In some embodiments, the polyanionic peptide is a peptide of the formula D₂₄. In some embodiments, the polyanionic peptide is a peptide of the formula D₂₅.

In some embodiments, the polyanionic peptide is a peptide of the formula D₁₀.

In some embodiments, the aspartate residues are consecutive. In some embodiments, the aspartate residues are discontinuous.

In some embodiments, the ratio of amino acids bearing a side-chain that is negatively-charged at physiological pH to the total quantity of amino acids in the polyanionic peptide is from about 0.5 to about 2.0.

In some embodiments, the TGF-β antagonist is bound to the targeting moiety directly, e.g., by a covalent bond, such as an amide bond, disulfide bridge, thioether bond, or carbon-carbon bond, among others. In some embodiments, the TGF-β antagonist is bound to the targeting moiety by way of a linker, such as a peptidic linker or a synthetic linker described herein.

In some embodiments, the TGF-β antagonist is bound to the N-terminus of a peptidic targeting moiety. For instance, in some embodiments, the C-terminus of a peptidic TGF-β antagonist is bound to the N-terminus of a peptidic moiety. In some embodiments, the TGF-β antagonist is bound to the C-terminus of the targeting moiety. For instance, in some embodiments, the N-terminus of a peptidic TGF-β antagonist is bound to the C-terminus of a peptidic moiety.

In some embodiments, the TGF-β antagonist is bound to the targeting moiety by way of an immunoglobulin Fc domain.

In some embodiments, the TGF-β antagonist is bound to the N-terminus of the immunoglobulin Fc domain and the targeting moiety is bound to the C-terminus of the immunoglobulin Fc domain. In some embodiments, the TGF-β antagonist is bound to the C-terminus of the immunoglobulin Fc domain and the targeting moiety is bound to the N-terminus of the immunoglobulin Fc domain. In some embodiments, the immunoglobulin is selected from the group consisting of human IgG, human IgA, human IgM, human IgE, and human IgD.

In another aspect, the invention provides a pharmaceutical composition containing the conjugate of any one of the above aspects or embodiments of the invention and a pharmaceutically acceptable excipient. In some embodiments, the conjugate is formulated for subcutaneous, intradermal, intramuscular, intraperitoneal, intravenous, intranasal, epidural, or oral administration.

In another aspect, the invention features a method of treating a human patient suffering from a disease associated with elevated TGF-β signaling by administering to the patient a therapeutically effective of a conjugate or pharmaceutical composition described herein. In some embodiments, the disease is a bone disease.

In some embodiments, the disease is osteogenesis imperfecta. For instance, the disease may be Type I osteogenesis imperfecta, Type II osteogenesis imperfecta, Type III osteogenesis imperfecta, Type IV osteogenesis imperfecta, Type V osteogenesis imperfecta, Type VI osteogenesis imperfecta, Type VII osteogenesis imperfecta, Type VIII osteogenesis imperfecta, Type IX osteogenesis imperfecta, Type X osteogenesis imperfecta, or Type XI osteogenesis imperfecta.

In some embodiments, the disease is renal osteodystrophy, hyperparathyroid induced bone disease, diabetic bone disease, osteoarthritis, and steroid induced bone disease, disuse osteoporosis, or Cerebral Palsy.

In another aspect, the invention features a method of treating a human patient suffering from a disease associated with elevated bone turnover by administering to the patient a therapeutically effective of a conjugate or pharmaceutical composition described herein. In some embodiments, the disease is McCune-Albright Syndrome, Gaucher Disease, Hyperoxaluria, Paget Disease of bone, or Juvenile Paget Disease. In some embodiments, the disease is metastatic bone cancer, such as a bone metastasis that is secondary to a cancer of the breast or prostate.

In some embodiments, the disease is osteoporosis, fibrous dysplasia, Calmurati-Engleman Disease, Marfan's Syndrome, osteoglophonic dysplasia, autosomal dominant osteopetrosis, osteoporosis-pseudoglioma syndrome, juvenile, geroderma osteodysplastica, osteogenesis imperfecta congenita, microcephaly, or cataracts. In some embodiments, the disease is pseudohypoparathyroidism, Cleidocranial Dysplasia, Dyskeratosis Congenita, Exudative Vitreoretinopathy 1, Schimmelpenning-Feuerstein-Mims Syndrome, Prader-Willi Syndrome, Achondrogenesis, Antley-Bixler Syndrome, Aspartylglucosaminuria, Celiac Disease, Cerebrooculofacioskeletal Syndrome 1, Lysinuric Protein Intolerance, neuropathy, dyskeratosis congenita, Ehlers-Danlos Syndrome, epiphyseal dysplasia, hyaline fibromatosis syndrome, Perrault Syndrome 1, hemochromatosis, homocystinuria (e.g., due to cystathionine beta-synthase deficiency), hypophosphatemic rickets with hypercalciuria, desbuquois dysplasia, multiple pterygium syndrome, lethal congenital contracture syndrome 1, mitochondrial DNA depletion Ssndrome 6 (hepatocerebral Type), Niemann-Pick Disease, osteopetrosis, porphyria, Rothmund-Thomson Syndrome, Wilson Disease, Dent Disease 1, occipital horn syndrome, hyperglycerolemia, hypophosphatemic rickets, Lowe Oculocerebrorenal Syndrome, renal tubulopathy, diabetes mellitus, cerebellar ataxia, vitamin D hydroxylation-deficient rickets, Warburg micro syndrome 1, Stuve-Wiedemann Syndrome, Blue Rubber Bleb Nevus syndrome, Singleton-Merten Syndrome, microcephalic osteodysplastic primordial dwarfism, dysosteosclerosis, Hallermann-Streiff Syndrome, Bruck Syndrome 1, multiple pterygium syndrome (e.g., X-Linked), spondylometaphyseal dysplasia with dentinogenesis imperfecta, Hall-Riggs Mental Retardation Syndrome, infantile multisystem neurologic disease with osseous fragility, acrocephalopolysyndactyly Type III, acroosteolysis, ACTH-independent macronodular adrenal hyperplasia, amino aciduria with mental deficiency, arthropathy, bone fragility (e.g., with craniosynostosis, ocular proptosis, hydrocephalus, and distinctive facial features), brittle cornea syndrome, cerebrotendinous xanthomatosis, Cri-Du-Chat Syndrome, dysplasia epiphysealis hemimelica, autosomal dominant Ehlers-Danlos Syndrome, familial osteodysplasia, Flynn-Aird Syndrome, geroderma osteodysplastica, glycogen storage disease Ia, Hutchinson-Gilford Progeria Syndrome, Infantile Systemic Hyalinosis, hypertrichotic osteochondrodysplasia, hyperzincemia with functional zinc depletion, hypophosphatasia, autosomal dominant hypophosphatemic rickets, X-linked recessive hypophosphatemic rickets, Lichtenstein Syndrome, macroepiphyseal dysplasia (e.g., with osteoporosis wrinkled skin, and agedappearance), Menkes Disease, Mental Retardation (e.g., X-Linked, Snyder-Robinson type), Jansen type metaphyseal chondrodysplasia, microspherophakia-metaphyseal dysplasia, morquio syndrome a, Morquio Syndrome B, ossified ear cartilages (e.g., with mental deficiency, muscle wasting, and osteocraniostenosis), osteoporosis and oculocutaneous hypopigmentation syndrome, osteoporosis-pseudoglioma syndrome, juvenile osteoporosis, osteosclerosis with ichthyosis and fractures, ovarian dysgenesis 1, ovarian dysgenesis 2, ovarian dysgenesis 3, ovarian dysgenesis 4, pituitary adenoma, polycystic lipomembranous osteodysplasia with sclerosing leukoencephalopathy, Prader-Willi Habitus, osteopenia, Okamoto type premature aging syndrome, Prieto X-linked mental retardation syndrome, pycnodysostosis, Pyle Disease, Reifenstein Syndrome, autosomal dominant distal renal tubular acidosis, Type 1 Schwartz-Jampel Syndrome, Type 2 Schwartz-Jampel Syndrome, Type 3 Schwartz-Jampel Syndrome, Type 4 Schwartz-Jampel Syndrome, X-linked spondyloepiphyseal dysplasia tarda, or Torg-Winchester Syndrome.

In some embodiments, the method includes administering the conjugate or pharmaceutical composition to the patient subcutaneously, intradermally, intramuscularly, intraperitoneally, intravenously, or orally, or by nasal or by epidural administration.

In another aspect, the invention features a kit containing a conjugate or pharmaceutical composition described herein as well as a package insert that instructs a user of the kit to treat a human patient suffering from a disease associated with elevated TGF-β signaling or elevated bone turnover (such as any of the foregoing diseases or conditions) by administering to the patient a therapeutically effective amount of the conjugate.

### Definitions

As used herein, the term “about” refers to a value that is within 10% above or below the value being described. For instance, the phrase “about 50 nM” refers to a value between and including 45 nM and 55 nM.

As used herein, the term “affinity” refers to the strength of a binding interaction between two molecules, such as a ligand and a receptor. The term “K_{d}”, as used herein, is intended to refer to the dissociation constant, which can be obtained, for example, from the ratio of the rate constant for the dissociation of the two molecules KO to the rate constant for the association of the two molecules (kₐ) and is expressed as a molar concentration (M). K_{d} values for peptide-protein interactions can be determined, e.g., using methods established in the art. Methods that can be used to determine the K_{d} of a peptide-protein interaction include surface plasmon resonance, e.g., through the use of a biosensor system such as a BIACORE® system, as well as fluorescence anisotropy and polarization methods and calorimetry techniques known in the art, such as isothermal titration calorimetry (ITC).

An “affinity-matured antibody” as used herein is an antibody or a fragment thereof with one or more amino acid substitutions in a variable region, such as the heavy chain variable region or light chain variable region, which results in improved affinity of the antibody for an antigen (e.g., TGF-β) as compared to a reference antibody, such as 1D11 or GC1008 described herein, that lacks the one or more amino acid substitutions. Methods of affinity-maturation of antibodies and antigen-binding fragments thereof are known in the art and are described, for instance, in Daugherty et al., Protein Engineering, Design and Selection 11:825-832 (1998); Yang et al., Journal of Molecular Biology 254:392-403 (1995); Lippow et al., Nature Biotechnology 25:1171-1176 (2007); Gram et al., Proceedings of the National Academy of Sciences of the United States of America 89:3576-3580 (1992); and Hawkins et al., Journal of Molecular Biology 226:889-896 (1992), the disclosures of each of which are incorporated herein by reference as they pertain to techniques for preparing affinity-matured antibodies and antigen-binding fragments thereof.

As used herein, the term “antibody” refers to an immunoglobulin molecule that specifically binds to, or is immunologically reactive with, a particular antigen, and includes polyclonal, monoclonal, genetically engineered, optimized (e.g., affinity-matured), and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen binding fragments of antibodies, including, for example, Fab′, F(ab′)₂, Fab, Fv, rIgG, and scFv fragments. Unless otherwise indicated, the term “monoclonal antibody” (mAb) is meant to include both intact molecules, as well as antibody fragments (including, for example, Fab and F(ab′)₂ fragments) that are capable of specifically binding to a target protein. As used herein, the Fab and F(ab′)₂ fragments refer to antibody fragments that lack the Fc fragment of an intact antibody. Examples of these antibody fragments are described herein.

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be, for example, a Fab, F(ab′)₂, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment containing two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment that consists of a V_{H} domain (see, e.g., Ward et al., Nature 341:544-546, 1989); (vii) a dAb which consists of a V_{H} or a V_{L} domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “anti-TGF-β antibody” refers to a protein or peptide-containing molecule that includes at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework region, or any portion thereof, that is capable of specifically binding to TGF-β, such as a TGF-β1, TGF-β2, or TGF-β3 isoform thereof. Anti-TGF-β antibodies also include antibody-like protein scaffolds, such as the tenth fibronectin type III domain (¹⁰Fn3), which contains BC, DE, and FG structural loops similar in structure and solvent accessibility to antibody CDRs. The tertiary structure of the ¹⁰Fn3 domain resembles that of the variable region of the IgG heavy chain, and one of skill in the art can graft, for example, the CDRs of an anti-TGF-β monoclonal antibody onto the fibronectin scaffold by replacing residues of the BC, DE, and FG loops of ¹⁰Fn3 with residues from the CDRH-1, CDRH-2, or CDRH-3 regions of an anti-TGF-β monoclonal antibody.

As used herein, the term “bispecific antibody” refers to, for example, a monoclonal, often a human or humanized antibody that is capable of binding at least two different antigens. For instance, one of the binding specificities can be directed toward TGF-β and the other can specifically bind a different antigen.

As used herein, the term “bone turnover” refers to the dual processes of resorption (e.g., by osteoclasts) and redeposition (e.g., by osteoblasts) of calcium and other minerals that comprise bone tissue. In healthy individuals, the net effect of these processes is the maintenance of a constant mineral balance. In normal growing bones, the mineral deposition is in equilibrium with the mineral resorption, whereas in certain pathological conditions, bone resorption exceeds bone deposition. As used herein, the term “elevated bone turnover” in the context of a patient suffering from a pathological disease or condition refers to an increase in the rate of bone resorption and redeposition relative to a reference level, such as the rate of bone resorption and redeposition in a healthy subject not suffering from the disease or condition or the rate of resorption and redeposition in the subject of interest as measured prior to the subject being diagnosed with the disease or condition. Methods for assessing bone turnover include, for instance, measuring the concentration of one or more biomarkers of bone turnover in a subject, such as serum and bone alkaline phosphatase, serum osteocalcin (sOC), serum type I collagen C-telopeptide breakdown products (sCTX), urinary free-deoxypyridinoline (ufDPD), and urinary cross-linked N-telopeptides of type I collagen (uNTX) and comparing the concentration of the one or more biomarkers to that of a healthy subject, as described, for instance, in Braga et al. Bone 34:1013-1016 (2004), the disclosure of which is incorporated herein by reference as it pertains to biomarkers for assessing bone turnover.

As used herein in the context of conjugates, the term “bound to” refers to the covalent joining of one molecule, such as an antibody, protein, polypeptide, or domain thereof (e.g., a TGF-β antagonist antibody, protein, polypeptide, or domain thereof), to another molecule, such as another antibody, protein, polypeptide, or domain thereof (e.g., a bone-targeting moiety, such as an antibody, protein, polypeptide, or domain thereof that binds collagen or hydroxyapatite). Two molecules that are “bound to” one another as described herein may be directly bound to one another, for instance, without an intervening linker. Alternatively, two molecules that are “bound to” one another may be bound by way of a linker. Exemplary linkers include synthetic linkers containing coupling moieties listed in Table 9, herein, as well as peptidic linkers, such as those that contain one or more glycine, serine, and/or threonine residues. Additional examples of linkers that may be used in conjunction with the compositions and methods described herein include immunoglobulin Fc domains, as well as fragments thereof.

As used herein, the term “complementarity determining region” (CDR) refers to a hypervariable region found both in the light chain and the heavy chain variable domains of an antibody. The more highly conserved portions of variable domains are referred to as framework regions (FRs). The amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may contain modifications in these hybrid hypervariable positions. The variable domains of native heavy and light chains each contain four framework regions that primarily adopt a β-sheet configuration, connected by three CDRs, which form loops that connect, and in some cases form part of, the β-sheet structure. The CDRs in each chain are held together in close proximity by the framework regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest, National Institute of Health, Bethesda, Md., 1987). As used herein, numbering of immunoglobulin amino acid residues is performed according to the immunoglobulin amino acid residue numbering system of Kabat et al., unless otherwise indicated.

As used herein, the term “conjugate” refers to a molecule containing two or more regions from distinct sources that are ligated together (e.g., by a covalent bond) to form a single compound.

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in table 1 below.

TABLE 1
Representative physicochemical properties of naturally-occurring
amino acids

| | | | | Electrostatic | |
|---|---|---|---|---|---|
| | 3 | | Side- | character at | |
| | Letter | 1 Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term “diabody” refers to a bivalent antibody containing two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabody” refers to trivalent antibodies containing three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structures, peptides configured in this way typically trimerize so as to position the V_{H} and V_{L} domains of neighboring peptide chains spatially proximal to one another (see, for example, Holliger et al., Proc. Natl. Acad. Sci. USA 90:6444-48, 1993).

As used herein, a “dual variable domain immunoglobulin” (“DVD-Ig”) refers to an antibody that combines the target-binding variable domains of two monoclonal antibodies via linkers to create a tetravalent, dual-targeting single agent (see, for example, Gu et al., Meth. Enzymol., 502:25-41, 2012).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “framework region” or “FW region” includes amino acid residues that are adjacent to the CDRs of an antibody or antigen-binding fragment thereof. FW region residues may be present in, for example, human antibodies, humanized antibodies, monoclonal antibodies, antibody fragments, Fab fragments, single chain antibody fragments, scFv fragments, antibody domains, and bispecific antibodies, among others.

As used herein, the term “human antibody” refers to an antibody in which substantially every part of the protein (for example, all CDRs, framework regions, C_{L}, C_{H} domains (e.g., C_{H}1, C_{H}2, C_{H}3), hinge, and V_{L} and V_{H} domains) is substantially non-immunogenic in humans, with only minor sequence changes or variations. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598).

As used herein, the term “humanized” antibody refers to a non-human antibody that contains minimal sequences derived from non-human immunoglobulin. In general, a humanized antibody contains substantially all of at least one, and typically two, variable domains, in which all or substantially all of the CDR regions correspond to those of a non-human immunoglobulin. All or substantially all of the FW regions may also be those of a human immunoglobulin sequence. The humanized antibody can also contain at least a portion of an immunoglobulin constant region (Fc), typically that of a human immunoglobulin consensus sequence. Methods of antibody humanization are known in the art and have been described, for example, in Riechmann et al., Nature 332:323-7, 1988; U.S. Pat. Nos. 5,530,101; 5,585,089; 5,693,761; 5,693,762; and 6,180,370.

As used herein, the term “mineral” in the context of a bone-targeting moiety refers to an inorganic ion, complex, or compound, comprised of inorganic elements, that is present in bone. Exemplary minerals include, without limitation, Ca²⁺, PO₄³⁻, OH⁻, and other trace inorganic elements. The mineral can include, for instance, such compounds as crystalline, nanocrystalline or amorphous hydroxyapatite (Ca₁₀(PO₄)₆(OH)₂), calcium carbonate, and calcium phosphates with solubility behavior, under acidic and basic conditions, similar to that of hydroxyapatite, including, but not limited to, dicalcium phosphate, tricalcium phosphate, octacalcium phosphate or calcium phosphates.

As used herein, the term “monoclonal antibody” refers to an antibody that is derived from a single clone, including any eukaryotic, prokaryotic, or phage clone, and not the method by which it is produced.

As used herein, the term “neutralize” refers to the reduction or prevention of the activity of a molecule due to the action of an antagonistic substance. For instance, a substance capable of neutralizing TGF-β, such as a TGF-β antagonist antibody, protein, peptide, or small molecule described herein, is one that is capable of suppressing TGF-β signaling and/or capable of suppressing the effects of TGF-β on a particular cell type, such as an osteoblast or osteoclast, for instance, as described herein. Exemplary methods of determining the extent to which a substance neutralizes TGF-β include the osteoblast viability assays, osteoblast mineralization assays, collagen deposition assays, and alkaline phosphatase activity assays described in the Examples, below.

As used herein, the term “optimized antibody” refers to an antibody that features one or more amino acid substitutions, deletions, and/or insertions relative to a reference antibody sequence, such as the sequence of a reference antibody described herein (e.g., anti-TGF-β antibody 1D11 or GC1008), that result in an improvement in one or more pharmacological properties of the antibody. Exemplary features of an optimized antibody that may be improved relative to a reference antibody from which the optimized antibody is prepared include, without limitation, enhanced target affinity (e.g., affinity for TGF-β or one or more isoforms thereof), heightened target specificity, reduced aggregation propensity in aqueous solution, enhanced yield from recombinant expression, reduced immunogenicity, and improved thermal stability, among others. Examples of alterations in the amino acid sequence of a reference antibody that may result in an optimized antibody include those that replace amino acids that are prone to post-translational modification, such as cysteine residues that are sensitive to disulfide bond formation, as well as asparagine and glutamine residues susceptible to deamination and glycosylation, with isosteric amino acids of higher chemical stability. Conservative amino acid substitutions that can be used to effectuate one or more of the foregoing improvements are described in Table 1, below. Optimized antibodies can be developed, for instance, by a service specializing therein, such as ADIMAB™ (Lebanon, N.H.), and methods that can be used to produce optimized antibodies are described, for example, in WO 2009/036379 and U.S. Pat. No. 9,354,228, the disclosures of each of which are incorporated herein by reference as they pertain to techniques for preparing optimized antibodies from a reference antibody.

As used herein, a “peptide” refers to a single-chain polyamide containing a plurality of amino acid residues, such as naturally-occurring and/or non-natural amino acid residues, that are consecutively bound by amide bonds. Examples of peptides include shorter fragments of full-length proteins, such as full-length naturally-occurring proteins.

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

As used herein, the term “pharmaceutical composition” refers to a mixture containing a therapeutic compound, such as a conjugate described herein, to be administered to a subject, such as a mammal, e.g., a human, in order to prevent, treat or control a particular disease or condition (such as a disease or condition associated with elevated TGF-β activity or elevated bone turnover described herein) affecting or that may affect the mammal.

As used herein, the term “pharmaceutically acceptable” refers to the suitability of a carrier or vehicle for use in mammals, including humans, without undue toxicity, incompatibility, instability, irritation, allergic response, and the like, commensurate with a reasonable benefit/risk ratio.

As used herein, the term “polyanionic peptide” refers to a peptide that has a net negative charge at physiological pH as assessed by determining the quantity of amino acid residues within the peptide that have side-chains that are negatively charged at physiological pH, such as aspartate and glutamate residues as described in Table 1, above. Polyanionic peptides contain two or more amino acid residues that have a side-chain that exhibits a formal −1 charge at physiological pH and/or one or more amino acid residues that have a side-chain that exhibits a formal −2 charge or less. The formal charge of an amino acid residue at a particular pH, such as physiological pH (7.4) can be determined using the Henderson-Hasselbalch equation, pH=pKa+log[A−]/[HA], as applied to the side-chain functional group of the amino acid of interest, wherein “HA” designates the protonated form of the side-chain substituent and “A-” designates the deprotonated form of the side-chain substituent. It will be appreciated by one of skill in the art that the Henderson-Hasselbalch equation may be applied multiple times to the same amino acid for those that contain side-chains that undergo more than one ionization at the pH of interest (e.g., pH of 7.4), such as those that contain a phosphate substituent, among others. The formal charge of an amino acid as described herein refers to the charge of the predominant form (i.e., the form present in the highest quantity at chemical equilibrium) of the amino acid side chain substituent (e.g., “HA” or “A-”) as determined by the Henderson-Hasselbalch equation.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject (e.g., a human subject, such as a human subject suffering from a disease or condition associated with elevated TGF-β activity or elevated bone turnover as described herein).

As used herein, the term “scFv” refers to a single chain Fv antibody in which the variable domains of the heavy chain and the light chain from an antibody have been joined to form one chain. scFv fragments contain a single polypeptide chain that includes the variable region of an antibody light chain (V_{L}) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the V_{L} and V_{H} regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody.

As used herein, the phrases “specifically binds” and “binds” refer to a binding reaction which is determinative of the presence of a particular protein, mineral, or other particular compound in a heterogeneous population of proteins and other biological molecules that is recognized, e.g., by a ligand with particularity. A ligand (e.g., a protein, peptide, or small molecule) that specifically binds to a protein will bind to the protein, e.g., with a K_{D} of less than 100 μM. For example, a peptide (e.g., a TGF-β-binding peptide, a collagen-binding peptide, or a hydroxyapatite-binding peptide) that specifically binds to a protein (e.g., TGF-β) may bind to the protein with a K_{D} of up to 1 μM (e.g., between 1 pM and 1 μM). A variety of assay formats may be used to determine the affinity of a ligand (e.g., a peptide, such as a TGF-β-binding peptide, collagen-binding peptide, or hydroxyapatite-binding peptide) for a specific protein (e.g., TGF-β or collagen) or mineral (e.g., hydroxyapatite). For example, solid-phase ELISA assays are routinely used to identify ligands that specifically bind a particular protein. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

As used herein, the terms “subject” and “patient” are interchangeable and refer to an organism that receives treatment for a particular disease or condition as described herein. Examples of subjects and patients include mammals, such as humans, receiving treatment for diseases or conditions, such as conditions associated with elevated TGF-β activity or elevated bone turnover.

As used herein, the term “targeting moiety” refers to a compound, such as a peptide, that specifically binds an endogenous component that is expressed in a particular tissue type. For instance, bone-targeting moieties described herein contain a compound, such as a peptide, that specifically binds to an endogenous component of osseous tissue. In the context of bone-targeting moieties, the endogenous component of osseous tissue may be, for example, a protein, such as collagen, or a mineral, such as hydroxyapatite. Due to their specific binding affinity, targeting moieties can be capable of localizing a compound of interest, such as a TGF-β antagonist, to a particular tissue of interest, such as bone.

As used herein, the term “TGF-β antagonist” refers to a compound (e.g., a protein, peptide, antibody, or small molecule) capable of inhibiting TGF-β signaling. A TGF-β antagonist may contain a protein, peptide, or antibody and, optionally, one or more non-peptidic molecules. A TGF-β antagonist may contain, consist of, or consist essentially of a TGF-β-binding protein, peptide, antibody, or small molecule, which refers to a protein, peptide, antibody, or small molecule capable of binding TGF-β. Alternatively, a TGF-β antagonist may contain, consist of, or consist essentially of a protein, peptide, antibody, or small molecule that binds a TGF-β receptor so as to inhibit the ability of TGF-β to bind the receptor, thereby attenuating TGF-β signaling. Exemplary TGF-β antagonists that bind TGF-β and exemplary TGF-β antagonists that bind TGF-β receptors are known in the art and are described herein.

As used herein, the term “TGF-β signaling” refers to the endogenous signal transduction cascade by which TGF-β potentiates the intracellular activity of the TGF-β receptor so as to effect one or more biological responses. TGF-β signaling encompasses the TGF-β-mediated stimulation of a TGF-β receptor and concomitant phosphorylation and activation of receptor-associated Smad proteins. TGF-β signaling includes the translocation of one or more Smad transcription factors to the nucleus, for example, by way of an interaction between a Smad protein and nucleoporins. TGF-β signaling encompasses the release of one or more Smad protein from Smad Anchor for Receptor Activation (SARA), which sequesters Smad proteins in the cytoplasm and prevents their translocation into the nucleus. As used herein, the term “elevated TGF-β activity” in the context of a patient suffering from a pathological disease or condition refers to an increase in TGF-β signaling relative to a reference level, such as TGF-β signaling in a healthy subject not suffering from the disease or condition or TGF-β signaling in the subject of interest as measured prior to the subject being diagnosed with the disease or condition. Methods for assessing TGF-β signaling are known in the art and include, for instance, measuring the extent of transcription of a gene of interest under the control of a promoter regulated by a transcription factor (e.g., a Smad protein) that is activated by the TGF-β signal transduction cascade, as well as measuring the concentration or relative level of one or more phosphorylated Smad transcription factors.

As used herein, the term “therapeutically effective amount” of a therapeutic agent, such as a conjugate described herein, refers to an amount that is sufficient, when administered to a subject suffering from or susceptible to a disease, disorder, and/or condition, (e.g., a disease, disorder, and/or condition associated with elevated TGF-β activity and/or bone turnover as described herein) to treat, prevent, and/or delay the onset of one or more symptom(s) of the disease, disorder, and/or condition.

As used herein, the terms “treat” or “treatment” in the context of a subject suffering from a disease or condition associated with elevated TGF-β activity and/or bone turnover refer to treatment, for instance, by administration of a conjugate containing a TGF-β antagonist and a bone-targeting moiety as described herein, with the intention of alleviating a phenotype associated with the disease or condition. For instance, exemplary forms of treatment include administration of a conjugate, such as a conjugate described herein, to a subject suffering from a bone disorder, such as osteogenesis imperfecta (e.g., osteogenesis imperfecta of Types I-XI) so as to reduce the progression of the disease or attenuate the severity of one or more symptoms associated with the disease, such as the propensity of the subject to suffer from recurring bone fractures.

### BRIEF DESCRIPTION OF THE FIGURES

FIG. 1 is a graph demonstrating the effect of anti-TGF-β antibody GC1008 conjugated to a decaaspartate hydroxyapatite-binding peptide on the viability of cultured mouse calvarial pre-osteoblasts (MC3T3-E1) as assessed by analyzing the incorporation of 3-(4,5-dimethyl-2-thiazolyl)-2,5-diphenyl-2H-tetrazolium bromide (MTT, SIGMA-ALDRICH™) into viable cells at various time points. Values along the y-axis designate the absorbance of the cultured cells at 595 nm, indicative of the reduction of the tetrazolium dye to a formazan by mitochondrial reductases present within viable cells. Values along the x-axis represent the time point during the cell culture period at which the absorbance measurement was recorded. MC3T3-E1 cells were cultured in the presence of ascorbic acid (AA) and β-glycerophosphate ((3GP). As described in the Examples below, TGF-β exerted a dose-dependent adverse effect on osteoblast viability. Cell viability was rescued in a dose-dependent fashion upon the addition of anti-TGF-β antibody GC1008 conjugated to decaaspartate (represented as “D10 Tagged Ab” in FIG. 1).

FIG. 2A provides a series of images demonstrating the effects of anti-TGF-β antibody GC1008 conjugated to a decaaspartate hydroxyapatite-binding peptide on mineralization by osteoblast cultures obtained by the induced differentiation of MC3T3 cells as assessed by silver phosphate precipitation and silver deposition (Von Kossa staining, as described in the Examples below). FIG. 2B is a table demonstrating the effects of anti-TGF-β antibody GC1008 conjugated to a decaaspartate hydroxyapatite-binding peptide on mineralization by MC3T3-E1 osteoblast cultures as assessed by Von Kossa staining. FIG. 2B reports quantitatively the percentage of mineralized area observed for the conditions shown in FIG. 2A, as well as two additional culture conditions.

FIG. 3 is a graph demonstrating the effect of anti-TGF-β antibody GC1008 conjugated to a decaaspartate hydroxyapatite-binding peptide on calcium deposition by MC3T3-E1 osteoblast cultures. Values along the y-axis represent the percentage of calcium deposition observed for the conditions specified along the x-axis relative to the quantity of calcium deposition recorded for MC3T3-E1 osteoblasts cultured in the presence of ascorbic acid and 13GP alone.

FIG. 4 is a graph demonstrating the effect of anti-TGF-β antibody GC1008 conjugated to a decaaspartate hydroxyapatite-binding peptide on collagen deposition by MC3T3-E1 osteoblast cultures. Values along the y-axis represent the percentage of collagen deposition observed for the conditions specified along the x-axis relative to the quantity of calcium deposition recorded for MC3T3-E1 osteoblasts cultured in the presence of ascorbic acid and 13GP alone.

FIG. 5 is a graph demonstrating the effect of anti-TGF-β antibody GC1008 conjugated to a decaaspartate hydroxyapatite-binding peptide on alkaline phosphatase activity in MC3T3-E1 osteoblast cultures as assessed by spectrophotometric monitoring the hydrolysis of a p-nitrophenylphosphate substrate. Values along the y-axis represent alkaline phosphatase activity in terms of activity units per milligram of protein.

FIG. 6 is a graph demonstrating the relative binding affinities of anti-TGF-β antibody 1D11 alone and GC1008 conjugated to a decaaspartate hydroxyapatite-binding peptide for hydroxyapatite. Values along the y-axis represent the percentage of antibody bound to hydroxyapatite crystals following incubation and centrifugation, as described in the Examples below.

FIG. 7A is a surface plasmon resonance sensorgram illustrating the binding of anti-TGF-β antibody 1D11, both in its unmodified form (“No D10 tag”) and in the form of its humanized variant (GC1008) conjugated to a decaaspartate tag (“D10-Tagged Ab”), to TGF-β isoform TGF-β1. FIG. 7B is a surface plasmon resonance sensorgram illustrating the binding of anti-TGF-β antibody 1D11, both in its unmodified form (“No D10 tag”) and in the form of its humanized variant (GC1008) conjugated to a decaaspartate tag (“D10-Tagged Ab”), to TGF-β isoform TGF-β2. FIG. 7C is a surface plasmon resonance sensorgram illustrating the binding of anti-TGF-β antibody 1D11, both in its unmodified form (“No D10 tag”) and in the form of its humanized variant (GC1008) conjugated to a decaaspartate tag (“D10-Tagged Ab”), to TGF-β isoform TGF-β3.

### DETAILED DESCRIPTION

The invention provides therapeutic conjugates containing a TGF-β antagonist, such as a TGF-β antagonist protein, peptide, antibody, or small molecule, bound to a targeting moiety capable of localizing the TGF-β antagonist to osseous tissue, such as human bone. The conjugates described herein may contain a TGF-β antagonist that directly binds and inhibits TGF-β. In some embodiments, the conjugates contain a TGF-β antagonist that binds a TGF-β receptor, thereby impeding the ability of TGF-β to bind the receptor and potentiate signal transduction.

The present invention is based in part on the discovery that diseases associated with elevated TGF-β activity and/or elevated bone turnover can not only be treated with TGF-β antagonists, but the therapeutic efficacy of these compounds can be improved by localizing these functional agents to the site of the pathological bone tissue. This can be achieved by conjugating the TGF-β antagonist to a bone-targeting moiety, such as a collagen-binding domain or a hydroxyapatite-binding domain. The sections that follow provide a description of various TGF-β antagonists and bone-targeting moieties that can be incorporated into a therapeutic conjugate.

### TGF-β Antagonists

TGF-β antagonists that can be used in conjunction with the compositions and methods described herein include TGF-β antagonist peptides, such as those that bind TGF-β and inhibit TGF-β signal transduction. Exemplary peptides that bind TGF-β and inhibit TGF-β signaling include the ectodomain of the TGF-β co-receptor, CD109. This peptide is described in detail, for instance, in U.S. Pat. No. 7,173,002 and in US 2012/0079614, the disclosures of each of which are incorporated herein by reference in their entirety. This 1428-residue peptide, as well as fragments thereof, have been shown to inhibit TGF-β signaling in mammalian cells. Active forms of this peptide may contain a tyrosine (SEQ ID NO: 2) or serine (SEQ ID NO: 4) residue at position 703 within the CD109 sequence. Additionally, fragments of CD109, such as those containing the amino acid sequence of residues 21-1404 or 21-1428, may be used as TGF-β antagonist peptides in the context of the conjugates and methods described herein. Other fragments of CD109, such as those containing the amino acid sequence WIWLDTNMGYRIYQEFEVT (SEQ ID NO: 1) or WIWLDTNMGSRIYQEFEVT (SEQ ID NO: 3), which correspond to positions 694-712 of SEQ ID NO: 2 and SEQ ID NO: 4, respectively, may be used as TGF-β antagonists in the conjugates and methods described herein, as these sequences may contain a putative TGF-β binding site. Additional fragment of the CD109 peptide that can be used as a TGF-β antagonist peptide in the conjugates and methods described herein contain the amino acid sequence IDGVYDNAEYAERFMEENEGHIVDIHDFSLGSS (SEQ ID NO: 5), which corresponds to residues 651-683 of SEQ ID NO: 2, which may also contain a putative TGF-β binding site.

Additional fragments of CD109 that can be used in the conjugates and methods described herein include a 161-residue portion of this protein that has the amino acid sequence TMENVVHELELYNTGYYLGMFMNSFAVFQECGLWVLTDANLTKDYIDGVYDNAEYAERFM EENEGHIVDIHDFSLGSSPHVRKHFPETWIWLDTNMGSRIYQEFEVTVPDSITSWVATGF VISEDLGLGLTTTPVELQAFQPFFIFLNLPYSVIRGEEFAL (SEQ ID NO: 6). Additional peptidic fragments of CD109 that can be used in the conjugates and methods described herein may comprise at least 10, 15, 25, 50, 75, 100, 250, 500, 750, 1000, 1250, 1400 or more contiguous amino acids of SEQ ID NO:2. Exemplary CD109 fragments that may be used in conjunction with the compositions and methods described herein include those that contain a putative TGF-β binding site, such as peptides containing the amino acid sequence RKHFPETWIWLDTNMGYRIYQEFEV (SEQ ID NO: 7), which corresponds to residues 687-711 of SEQ ID NO: 2.

In addition to the above, peptide antagonists of TGF-β useful in conjunction with the compositions and methods described herein include those containing an amino acid sequence having at least 85% sequence identity (e.g., at least 85%, 90%, 95%, 97%, 99%, or greater) to one of the foregoing sequences and/or having one or more conservative amino acid substitutions with respect to one of the foregoing sequences.

The foregoing antagonistic TGF-β peptides are summarized in Table 2, below.

TABLE 2
Exemplary TGF-β antagonist peptide sequences based on CD109

| SEQ ID | |
|---|---|
| NO. | Amino acid sequence |
| 1 | WIWLDTNMGYRIYQEFEVT |
| 2 | MQGPPLLTAAHLLCVCTAALAVAPGPRFLVTAPGIIRPGGNVTIGVELLEHCPSQVT |
| | VKAELLKTASNLTVSVLEAEGVFEKGSFKTLTLPSLPLNSADEIYELRVTGRTQDEIL |
| | FSNSTRLSFETKRISVFIQTDKALYKPKQEVKFRIVTLFSDFKPYKTSLNILIKDPKSNL |
| | IQQWLSQQSDLGVISKTFQLSSHPILGDWSIQVQVNDQTYYQSFQVSEYVLPKFEV |
| | TLQTPLYCSMNSKHLNGTITAKYTYGKPVKGDVTLTFLPLSFWGKKKNITKTFKING |
| | SANFSFNDEEMKNVMDSSNGLSEYLDLSSPGPVEILTTVTESVTGISRNVSTNVFFK |
| | QHDYIIEFFDYTTVLKPSLNFTATVKVTRADGNQLTLEERRNNVVITVTQRNYTEYW |
| | SGSNSGNQKMEAVQKINYTVPQSGTFKIEFPILEDSSELQLKAYFLGSKSSMAVHSL |
| | FKSPSKTYIQLKTRDENIKVGSPFELVVSGNKRLKELSYMVVSRGQLVAVGKQNST |
| | MFSLTPENSWTPKACVIVYYIEDDGEIISDVLKIPVQLVFKNKIKLYWSKVKAEPSEK |
| | VSLRISVTQPDSIVGIVAVDKSVNLMNASNDITMENVVHELELYNTGYYLGMFMNSF |
| | AVFQECGLWVLTDANLTKDYIDGVYDNAEYAERFMEENEGHIVDIHDFSLGSSPHV |
| | RKHFPETWIWLDTNMGYRIYQEFEVTVPDSITSWVATGFVISEDLGLGLTTTPVELQ |
| | AFQPFFIFLNLPYSVIRGEEFALEITIFNYLKDATEVKVIIEKSDKFDILMTSNEINATGH |
| | QQTLLVPSEDGATVLFPIRPTHLGEIPITVTALSPTASDAVTQMILVKAEGIEKSYSQS |
| | ILLDLTDNRLQSTLKTLSFSFPPNTVTGSERVQITAIGDVLGPSINGLASLIRMPYGCG |
| | EQNMINFAPNIYILDYLTKKKQLTDNLKEKALSFMRQGYQRELLYQREDGSFSAFGN |
| | YDPSGSTWLSAFVLRCFLEADPYIDIDQNVLHRTYTWLKGHQKSNGEFWDPGRVIH |
| | SELQGGNKSPVTLTAYIVTSLLGYRKYQPNIDVQESIHFLESEFSRGISDNYTLALITY |
| | ALSSVGSPKAKEALNMLTWRAEQEGGMQFWVSSESKLSDSWQPRSLDIEVAAYA |
| | LLSHFLQFQTSEGIPIMRWLSRQRNSLGGFASTQDTTVALKALSEFAALMNTERTNI |
| | QVTVTGPSSPSPLAVVQPTAVNISANGFGFAICQLNVVYNVKASGSSRRRRSIQN |
| | QEAFDLDVAVKENKDDLNHVDLNVCTSFSGPGRSGMALMEVNLLSGFMVPSEAIS |
| | LSETVKKVEYDHGKLNLYLDSVNETQFCVNIPAVRNFKVSNTQDASVSIVDYYEPR |
| | RQAVRSYNSEVKLSSCDLCSDVQGCRPCEDGASGSHHHSSVIFIFCFKLLYFMEL |
| | WL |
| 3 | WIWLDTNMGSRIYQEFEVT |
| 4 | MQGPPLLTAAHLLCVCTAALAVAPGPRFLVTAPGIIRPGGNVTIGVELLEHCPSQVT |
| | VKAELLKTASNLTVSVLEAEGVFEKGSFKTLTLPSLPLNSADEIYELRVTGRTQDEIL |
| | FSNSTRLSFETKRISVFIQTDKALYKPKQEVKFRIVTLFSDFKPYKTSLNILIKDPKSNL |
| | IQQWLSQQSDLGVISKTFQLSSHPILGDWSIQVQVNDQTYYQSFQVSEYVLPKFEV |
| | TLQTPLYCSMNSKHLNGTITAKYTYGKPVKGDVTLTFLPLSFWGKKKNITKTFKING |
| | SANFSFNDEEMKNVMDSSNGLSEYLDLSSPGPVEILTTVTESVTGISRNVSTNVFFK |
| | QHDYIIEFFDYTTVLKPSLNFTATVKVTRADGNQLTLEERRNNVVITVTQRNYTEYW |
| | SGSNSGNQKMEAVQKINYTVPQSGTFKIEFPILEDSSELQLKAYFLGSKSSMAVHSL |
| | FKSPSKTYIQLKTRDENIKVGSPFELVVSGNKRLKELSYMVVSRGQLVAVGKQNST |
| | MFSLTPENSWTPKACVIVYYIEDDGEIISDVLKIPVQLVFKNKIKLYWSKVKAEPSEK |
| | VSLRISVTQPDSIVGIVAVDKSVNLMNASNDITMENVVHELELYNTGYYLGMFMNSF |
| | AVFQECGLWVLTDANLTKDYIDGVYDNAEYAERFMEENEGHIVDIHDFSLGSSPHV |
| | RKHFPETWIWLDTNMGSRIYQEFEVTVPDSITSWVATGFVISEDLGLGLTTTPVELQ |
| | AFQPFFIFLNLPYSVIRGEEFALEITIFNYLKDATEVKVIIEKSDKFDILMTSNEINATGH |
| | QQTLLVPSEDGATVLFPIRPTHLGEIPITVTALSPTASDAVTQMILVKAEGIEKSYSQS |
| | ILLDLTDNRLQSTLKTLSFSFPPNTVTGSERVQITAIGDVLGPSINGLASLIRMPYGCG |
| | EQNMINFAPNIYILDYLTKKKQLTDNLKEKALSFMRQGYQRELLYQREDGSFSAFGN |
| | YDPSGSTWLSAFVLRCFLEADPYIDIDQNVLHRTYTWLKGHQKSNGEFWDPGRVIH |
| | SELQGGNKSPVTLTAYIVTSLLGYRKYQPNIDVQESIHFLESEFSRGISDNYTLALITY |
| | ALSSVGSPKAKEALNMLTWRAEQEGGMQFWVSSESKLSDSWQPRSLDIEVAAYA |
| | LLSHFLQFQTSEGIPIMRWLSRQRNSLGGFASTQDTTVALKALSEFAALMNTERTNI |
| | QVTVTGPSSPSPLAVVQPTAVNISANGFGFAICQLNVVYNVKASGSSRRRRSIQNQ |
| | EAFDLDVAVKENKDDLNHVDLNVCTSFSGPGRSGMALMEVNLLSGFMVPSEAISLS |
| | ETVKKVEYDHGKLNLYLDSVNETQFCVNIPAVRNFKVSNTQDASVSIVDYYEPRRQ |
| | AVRSYNSEVKLSSCDLCSDVQGCRPCEDGASGSHHHSSVIFIFCFKLLYFMELWL |
| 5 | IDGVYDNAEYAERFMEENEGHIVDIHDFSLGSS |
| 6 | TMENVVHELELYNTGYYLGMFMNSFAVFQECGLWVLTDANLTKDYIDGVYDNAEY |
| | AERFMEENEGHIVDIHDFSLGSSPHVRKHFPETWIWLDTNMGSRIYQEFEVTVPDSI |
| | TSWVATGFVISEDLGLGLTTTPVELQAFQPFFIFLNLPYSVIRGEEFAL |
| 7 | RKHFPETWIWLDTNMGYRIYQEFEV |

In addition to the above, peptide antagonists capable of binding TGF-β for use with the compositions and methods described herein include those described in U.S. Pat. No. 7,723,473, the disclosure of which is incorporated herein by reference in its entirety, as well as peptide antagonists of TGF-β containing an amino acid sequence having at least 85% sequence identity (e.g., at least 85%, 90%, 95%, 97%, 99%, or greater) to one of these sequences and/or having one or more conservative amino acid substitutions with respect to one of these sequences. These TGF-β antagonists specifically bind to TGF-β receptors, which include type I, type II, type III and type V receptors. It has been shown that these peptides, some of which correspond in sequence to amino acid numbers 41-65 of TGF-β₁, TGF-β₂, and TGF-β₃, inhibit the binding of TGF-β₁, TGF-β₂, and TGF-β₃, to TGF-β receptors. These peptides have been shown to attenuate TGF-β-induced growth inhibition and TGF-β-induced expression of PAI-1. It has also been shown that the W/RXXD motif found within these peptide sequences determines the specificity of activity of the antagonist peptide. These TGF-β antagonist peptides are summarized in Table 3, below.

TABLE 3
Exemplary TGF-β antagonist peptides

| SEQ ID | |
|---|---|
| NO. | Amino acid sequence |
| 8 | ANFCLGPCPYIWSLDT |
| 9 | ANFCSGPCPYLRSADT |
| 10 | PYIWSLDTQY |
| 11 | PYLWSSDTQH |
| 12 | PYLRSADTTH |
| 13 | WSXD x = any AA |
| 14 | RSXD x = any AA |

Additional peptidic antagonists of TGF-β that can be used in conjunction with the compositions and methods described herein include peptide antagonists described in U.S. Pat. No. 7,057,013, the disclosure of which is incorporated herein by reference in its entirety, as well as peptide antagonists of TGF-β containing an amino acid sequence having at least 85% sequence identity (e.g., at least 85%, 90%, 95%, 97%, 99%, or greater) to one of these sequences and/or having one or more conservative amino acid substitutions with respect to one of these sequences. These TGF-β antagonist peptides are based on the structure of TGF-β or a TGF-β receptor, and were designed so as to disrupt the binding of endogenous TGF-β to a TGF-β receptor for the purposes of attenuating TGF-β signaling. These synthetic peptides are summarized in Tables 4 and 5, below.

TABLE 4
Exemplary TGF-β antagonist peptides that bind
TGF-β

| SEQ ID | |
|---|---|
| NO. | Amino acid sequence |
| 15 | TSLDATMIWTMM |
| 16 | SNPYSAFQVDIIVDI |
| 17 | TSLMIWTMM |
| 18 | TSLDASIIWAMMQN |
| 19 | SNPYSAFQVDITID |
| 20 | EAVLILQGPPYVSWL |
| 21 | LDSLSFQLGLYLSPH |

TABLE 5
Exemplary TGF-β antagonist peptides that bind
a TGF-β receptor

| SEQ ID | |
|---|---|
| NO. | Amino acid sequence |
| 51 | HANFCLGPCPYIWSL |
| 52 | FCLGPCPYIWSLDT |
| 53 | HEPKGYHANFCLGPCP |

Additional peptidic antagonists of TGF-β that can be used in conjunction with the compositions and methods described herein include peptide antagonists described in US 2009/0263410, the disclosure of which is incorporated herein by reference in its entirety, as well as peptide antagonists of TGF-β containing an amino acid sequence having at least 85% sequence identity (e.g., at least 85%, 90%, 95%, 97%, 99%, or greater) to one of these sequences and/or having one or more conservative amino acid substitutions with respect to one of these sequences. These peptides are summarized in Table 6, below.

TABLE 6
Exemplary TGF-β antagonist peptides that bind
TGF-β

| SEQ ID | |
|---|---|
| NO. | Amino acid sequence |
| 22 | TSLDASIIWAMMQN |
| 23 | KRIWFIPRSSWYERA |
| 24 | KRIWFIPRSSW |
| 25 | KRIWFIPRSSW (Amidated at C-terminus) |
| 26 | KRIWFIPRSSW (Acetylated at N-terminal K |
| | and amidated at C-terminus) |

Additional peptidic antagonists of TGF-β that can be used in conjunction with the compositions and methods described herein include peptide antagonists described in US 2011/0294734, the disclosure of which is incorporated herein by reference in its entirety, as well as peptide antagonists of TGF-β containing an amino acid sequence having at least 85% sequence identity (e.g., at least 85%, 90%, 95%, 97%, 99%, or greater) to one of these sequences and/or having one or more conservative amino acid substitutions with respect to one of these sequences. These peptides are summarized in Table 7, below.

TABLE 7
Exemplary TGF-β antagonist peptides

| SEQ ID | |
|---|---|
| NO. | Amino acid sequence |
| 27 | HANFCLGPCPYIWSL |
| 28 | FCLGPCPYIWSLDT |
| 29 | TSLDATMIWTMM |
| 30 | SNPYSAFQVDIIVDI |
| 31 | TSLMIWTMM |
| 32 | TSLDASIIWAMMQN |
| 33 | SNPYSAFQVDITID |
| 34 | EAVLILQGPPYVSWL |
| 35 | LDSLSFQLGLYLSPH |
| 36 | HEPKGYHANFCLGPCPYIWSLDT |
| 37 | WHKYFLRRPLSVRTR |
| 38 | RFFTRFPWHYHASRL |
| 39 | RKWFLQHRRMPVSVL |
| 40 | SGRRHLHRHHIFSLP |
| 41 | RLAHSHRHRSHVALT |
| 42 | PPYHRFWRGHRHAVQ |
| 43 | KRIWFIPRSSWYERA |
| 44 | MPLSRYWWLFSHRPR |
| 45 | KRIWFIPRSSWYER |
| 46 | KRIWFIPRSSWY |
| 47 | KRIWFIPRSSW |
| 48 | KRIWFIPRSSW (amidated at C-terminus) |
| 49 | KRIWFIPRSSW (acetylated at N-terminal K |
| | and amidated at C-terminus) |

Additional TGF-β antagonists useful in conjunction with the compositions and methods described herein include glycoprotein-A repetitions predominant protein (GARP), as well as well as peptide antagonists of TGF-β containing an amino acid sequence having at least 85% sequence identity (e.g., at least 85%, 90%, 95%, 97%, 99%, or greater) to this protein and/or having one or more conservative amino acid substitutions with respect to this protein. The antagonistic activity of this protein is described in detail, for example, in Wang et al., Molecular Biology of the Cell 23:1129-1139 (2012), the disclosure of which is incorporated herein by reference in its entirety. The amino acid sequence of GARP is shown below.

Glycoprotein-A repetitions predominant protein
(GARP):
(SEQ ID NO: 50)
MRPQILLLLALLTLGLAAQHQDKVPCKMVDKKVSCQVLGLLQVPSVLPPD
TETLDLSGNQLRSILASPLGFYTALRHLDLSTNEISFLQPGAFQALTHLE
HLSLAHNRLAMATALSAGGLGPLPRVTSLDLSGNSLYSGLLERLLGEAPS
LHTLSLAENSLTRLTRHTFRDMPALEQLDLHSNVLMDIEDGAFEGLPRLT
HLNLSRNSLTCISDFSLQQLRVLDLSCNSIEAFQTASQPQAEFQLTWLDL
RENKLLHFPDLAALPRLIYLNLSNNLIRLPTGPPQDSKGIHAPSEGWSAL
PLSAPSGNASGRPLSQLLNLDLSYNEIELIPDSFLEHLTSLCFLNLSRNC
LRTFEARRLGSLPCLMLLDLSHNALETLELGARALGSLRTLLLQGNALRD
LPPYTFANLASLQRLNLQGNRVSPCGGPDEPGPSGCVAFSGITSLRSLSL
VDNEIELLRAGAFLHTPLTELDLSSNPGLEVATGALGGLEASLEVLALQG
NGLMVLQVDLPCFICLKRLNLAENRLSHLPAWTQAVSLEVLDLRNNSFSL
LPGSAMGGLETSLRRLYLQGNPLSCCGNGWLAAQLHQGRVDVDATQDLIC
RFSSQEEVSLSHVRPEDCEKGGLKNINLIIILTFILVSAILLTTLAACCC
VRRQKFNQQYKA

Examples of additional TGF-β antagonists useful in conjunction with the compositions and methods described herein include monoclonal and polyclonal antibodies directed against one or more isoforms of TGF-β (U.S. Pat. No. 5,571,714 and PCT patent application WO 97/13844), TGF-β receptors, fragments thereof, derivatives thereof and antibodies directed against TGF-β receptors (U.S. Pat. Nos. 5,693,607, 6,008,011, 6,001,969 and 6,010,872 and PCT patent applications WO 92/00330, WO 93/09228, WO 95/10610 and WO 98/48024); latency associated peptide (WO 91/08291), large latent TGF-β (WO 94/09812), fetuin (U.S. Pat. No. 5,821,227), decorin and other proteoglycans such as biglycan, fibromodulin, lumican and endoglin (U.S. Pat. Nos. 5,583,103, 5,654,270, 5,705,609, 5,726,149, 5,824,655 5,830,847, 6,015,693 and PCT patent applications WO 91/04748, WO 91/10727, WO 93/09800 and WO 94/10187).

Particular TGF-β antagonists useful in conjunction with the compositions and methods described herein include anti-TGF-β antibody 1D11, as well as antigen-binding fragments thereof and human, humanized, and chimeric variants thereof. Anti-TGF-β antibody GC1008, a humanized variant of 1D11, is described in U.S. Pat. No. 9,958,486, the disclosure of which is incorporated herein by reference in its entirety. Anti-TGF-β antibody GC1008 contains the following complementarity determining regions (CDRs):
a. a CDR-H1 having the amino acid sequence SNVIS (SEQ ID NO: 327);
b. a CDR-H2 having the amino acid sequence GVIPIVDIANYAQRFKG (SEQ ID NO: 328);
c. a CDR-H3 having the amino acid sequence TLGLVLDAMDY (SEQ ID NO: 329);
d. a CDR-L1 having the amino acid sequence RASQSLGSSYLA (SEQ ID NO: 330);
e. a CDR-L2 having the amino acid sequence GASSRAP (SEQ ID NO: 331); and
f. a CDR-L3 having the amino acid sequence QQYADSPIT (SEQ ID NO: 332).

Anti-TGF-β antibody GC1008 contains a heavy chain variable region having the sequence of SEQ ID NO: 333, and a light chain variable region having the amino acid sequence of SEQ ID NO: 334, shown below:

GC1008 Heavy chain variable region amino acid
sequence
(SEQ ID NO: 333)
QVQLVQSGAEVKKPGSSVKVSCKASGYTFSSNVISWVRQAPGQGLEWMGG
VIPIVDIANYAQRFKGRVTITADESTSTTYMELSSLRSEDTAVYYCASTL
GLVLDAMDYWGQGTLVTVSS
GC1008 Light chain variable region amino acid
sequence
(SEQ ID NO: 334)
ETVLTQSPGTLSLSPGERATLSCRASQSLGSSYLAWYQQKPGQAPRLLIY
GASSRAPGIPDRFSGSGSGTDFTLTISRLEPEDFAVYYCQQYADSPITFG
QGTRLEIK

Anti-TGF-β antagonists useful in conjunction with the compositions and methods described herein include antibodies and antigen-binding fragments thereof containing one or more, or all, of the CDRs of GC1008, as well as those containing a set of CDRs that each have at least 85% sequence identity (e.g., at least 85%, 90%, 95%, 97%, 98%, 99%, or more, sequence identity) to the CDRs of GC1008, shown above. Exemplary anti-TGF-β antagonists useful in conjunction with the compositions and methods described herein include monoclonal antibodies and antigen-binding fragments thereof, polyclonal antibodies and antigen-binding fragments thereof, humanized antibodies and antigen-binding fragments thereof, bispecific antibodies and antigen-binding fragments thereof, optimized antibodies and antigen-binding fragments thereof (e.g., affinity-matured antibodies and antigen-binding fragments thereof), dual-variable immunoglobulin domains, single-chain Fv molecules (scFvs), diabodies, triabodies, nanobodies, antibody-like protein scaffolds, Fv fragments, Fab fragments, F(ab′)₂ molecules, and tandem di-scFVs, among others, such as those that have one or more, or all, of the CDRs of GC1008, as well as those containing a set of CDRs that each have at least 85% sequence identity (e.g., at least 85%, 90%, 95%, 97%, 98%, 99%, or more, sequence identity) to the CDRs of GC1008, shown above.

Additionally, antibodies and antigen-binding fragments thereof that may be used in conjunction with the compositions and methods described herein include those that bind the same epitope on TGF-β as murine antibody 1D11, its humanized counterpart, GC1008, and antibodies or antigen-binding fragments thereof that have the same set of CDRs as 1D11 and GC1008. Exemplary methods that can be used to determine whether an antibody or antigen-binding fragment thereof binds the same epitope on TGF-β as a reference antibody, such as 1D11 or GC1008, include competitive binding experiments, such as competitive ELISA experiments or other competitive binding assays known in the art. An antibody or antigen-binding fragment thereof is considered to bind the same epitope on TGF-β as a reference antibody, such as 1D11 or GC1008, if the antibody or antigen-binding fragment thereof competitively inhibits the binding of TGF-β to the reference antibody. Competitive binding experiments that can be used to determine whether an antibody or antigen-binding fragment thereof binds to the same epitope on TGF-β as a reference antibody or antigen-binding fragment thereof are described, for instance, in Nagata et al., Journal of Immunological Methods 292:141-155 (2004), the disclosure of which is incorporated herein by reference in its entirety.

Thus, antibodies and antigen-binding fragments thereof useful in conjunction with the compositions and methods described herein include those that competitively inhibit the binding of TGF-β to an antibody or antigen-binding fragment thereof that contains the following CDRs:
a. a CDR-H1 having the amino acid sequence SNVIS (SEQ ID NO: 327);
b. a CDR-H2 having the amino acid sequence GVIPIVDIANYAQRFKG (SEQ ID NO: 328);
c. a CDR-H3 having the amino acid sequence TLGLVLDAMDY (SEQ ID NO: 329);
d. a CDR-L1 having the amino acid sequence RASQSLGSSYLA (SEQ ID NO: 330);
e. a CDR-L2 having the amino acid sequence GASSRAP (SEQ ID NO: 331); and
f. a CDR-L3 having the amino acid sequence QQYADSPIT (SEQ ID NO: 332).

Antibodies and antigen-binding fragments thereof that may be used with the compositions and methods described herein include those that competitively inhibit the binding of TGF-β to an antibody or antigen-binding fragment thereof having the heavy chain variable region set forth in SEQ ID NO: 333 and/or the light chain variable region set forth in SEQ ID NO: 334.

Further examples of such antagonists include somatostatin (WO 98/08529), mannose-6-phosphate or mannose-1-phosphate (U.S. Pat. No. 5,520,926), prolactin (PCT patent application WO 97/40848), insulin-like growth factor II (PCT patent application WO 98/17304), IP-10 (PCT patent application WO97/00691), arg-gly-asp containing peptides (U.S. Pat. No. 5,958,411 and PCT patent application WO 93/10808 and), extracts of plants, fungi and bacteria (European patent application 813875, Japanese patent application 8119984 and U.S. Pat. No. 5,693,610), antisense oligonucleotides (U.S. Pat. Nos. 5,683,988, 5,772,995, 5,821,234 and 5,869,462 and PCT patent application WO 94/25588), and a host of other proteins involved in TGF-β signaling, including SMADs and MADs (European patent application EP 874046, PCT patent applications WO 97/31020, WO 97/38729, WO 98/03663, WO 98/07735, WO 98/07849, WO 98/45467, WO 98/53068, WO 98/55512, WO 98/56913, WO 98/53830, and WO 99/50296, and U.S. Pat. Nos. 5,834,248, 5,807,708 and 5,948,639) and Ski and Sno (G. Vogel, Science, 286:665 (1999) and Stroschein et al., Science, 286:771-74 (1999)) and fragments and derivatives of any of the above molecules that retain the ability to inhibit the activity of TGF-β.

Additional examples of TGF-β antagonists include small molecules that inhibit TGF-β signal transduction. These agents can be classified on the basis of the core molecular scaffolds of these molecules. For example, TGF-β signaling inhibitors may contain a dihydropyrrlipyrazole, imidazole, pyrazolopyridine, pyrazole, imidazopyridine, triazole, pyridopyrimidine, pyrrolopyrazole, isothiazole or oxazole functionality as the core structural fragment of the molecule. Some non-limiting examples of small molecule inhibitors of TGF-β signaling include ALKS inhibitor II (also referred to as E-616452), LY364947 (also referred to as ALKS Inhibitor I, TbR-I Inhibitor, Transforming Growth Factor-b Type I Receptor Kinase Inhibitor), A83-01, and DMH1, known in the art. Other examples of small molecule TGF-β antagonists that can be used in conjunction with the compositions and methods described herein include SB431542 (4-(5-Benzol[1,3]dioxol-5-yl-4-pyridin-2-yl-1H-imidazol-2-yl)-benzamide hydrate, 4-[4-(1,3-Benzodioxol-5-yl)-5-(2-pyridinyl)-1H-imidazol-2-yl]-benzamide hydrate, 4-[4-(3,4-Methylenedioxyphenyl)-5-(2-pyridyl)-1H-imidazol-2-yl]-benzamide hydrate, an Alk5 inhibitor), Galunisertib (LY2157299, an Alk5 inhibitor), LY2109761 (4-[2-[4-(2-pyridin-2-yl-5,6-dihydro-4H-pyrrolo[1,2-b]pyrazol-3-yl)quinolin-7-yl]oxyethyl]morpholine, an Alk5/TGFβRII inhibitor), SB525334 (6-[2-tert-butyl-5-(6-methylpyridin-2-yl)-1H-imidazol-4-yl]quinoxaline, an Alk5 inhibitor), GW788388 (N-(oxan-4-yl)-4-[4-(5-pyridin-2-yl-1H-pyrazol-4-yl)pyridin-2-yl]benzamide, an Alk5 inhibitor), K02288 (3-[6-amino-5-(3,4,5-trimethoxyphenyl)pyridin-3-yl]phenol, an Alk4/Alk5 inhibitor), SD-208 (2-(5-chloro-2-fluorophenyl)-N-pyridin-4-ylpteridin-4-amine, an Alk5 inhibitor), EW-7197 (N-((4-([1,2,4]triazolo[1,5-a]pyridin-6-yl)-5-(6-methylpyridin-2-yl)-1H-imidazol-2-yl)methyl)-2-fluoroaniline, an Alk4/Alk5 inhibitor), and LDN-212854 (5-[6-[4-(1-Piperazinyl)phenyl]pyrazolo[1,5-a]pyrimidin-3-yl]-quinoline, an Alk4/Alk5 inhibitor).

Additional examples of small molecule TGF-β antagonists include those that bind TGF-β receptors, such as 2-(3-(6-Methylpyridin-2-yl)-1H-pyrazol-4-yl)-1,5 napththyridine, [3-(Pyridin-2-yl)-4-(4-quinoyl)]-1H-pyrazole, and 3-(6-Methylpyridin-2-yl)-4-(4-quinolyl)-1-phenylthiocarbamoyl-1H-pyrazole. Other small molecule inhibitors include, but are not limited to, SB-431542, (4-[4-(1,3-Benzodioxol-5-yl)-5-(2-pyridinyl)-1H-imidazol-2-yl]-benzamide, described in Halder et al., Neoplasia 7(5):509-521 (2005)), SM16, a small molecule inhibitor of TGFβ receptor ALKS, the structure of which is shown below (Fu, K et al., Arteriosclerosis, Thrombosis and Vascular Biology 28(4):665 (2008)), SB-505124 (an Alk4/Alk5 inhibitor, structure shown below, described in Dacosta Byfield, S., et al., Molecular Pharmacology 65:744-752 (2004)), and 6-bromo-indirubin-3′-oxime (described in U.S. Pat. No. 8,298,825), the disclosures of each of which are incorporated herein by reference.

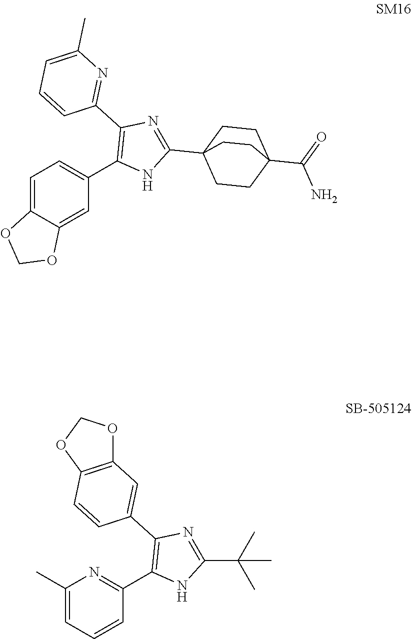 [SMILES: Cc1cccc(-c2nc(C34CCC(C(N)=O)(CC3)CC4)[nH]c2-c2ccc3c(c2)OCO3)n1 ; Cc1cccc(-c2[nH]c(C(C)(C)C)nc2-c2ccc3c(c2)OCO3)n1]

Additional examples of small molecule TGF-β antagonists include, without limitation, those that are described in, e.g., Callahan, J. F. et al., J. Med. Chem. 45:999-1001 (2002); Sawyer, J. S. et al., J. Med. Chem. 46:3953-3956 (2003); Gellibert, F. et al., J. Med. Chem. 47:4494-4506 (2004); Tojo, M. et al., Cancer Sci. 96:791-800 (2005); Valdimarsdottir, G. et al., APMIS 113:773-389 (2005); Petersen et al., Kidney International 73:705-715 (2008); Yingling, J. M. et al., Nature Rev. Drug Disc. 3:1011-1022 (2004); Byfield, S. D. et al., Mol. Pharmacol., 65:744-752 (2004); Dumont, N, et al., Cancer Cell 3:531-536 (2003); WO 2002/094833; WO 2004/026865; WO 2004/067530; WO 209/032667; WO 2004/013135; WO 2003/097639; WO 2007/048857; WO 2007/018818; WO 2006/018967; WO 2005/039570; WO 2000/031135; WO 1999/058128; U.S. Pat. Nos. 6,509,318; 6,090,383; 6,419,928; 7,223,766; 6,476,031; 6,419,928; 7,030,125; 6,943,191; US 2005/0245520; US 2004/0147574; US 2007/0066632; US 2003/0028905; US 2005/0032835; US 2008/0108656; US 2004/015781; US 2004/0204431; US 2006/0003929; US 2007/0155722; US 2004/0138188; and US 2009/0036382, the disclosures of each which are incorporated by reference as they pertain to TGF-β antagonists.

### Collagen-Binding Domains and Hydroxyapatite-Binding Domains

A variety of collagen-binding domains and hydroxyapatite binding domains can be used in conjunction with the compositions and methods described herein. For instance, a variety of peptides with collagen-binding activity have been described in U.S. Pat. No. 8,450,272, the disclosure of which is incorporated herein by reference in its entirety. Exemplary collagen-binding peptides described therein are summarized below.

(SEQ ID NO: 54)
Pro Val Tyr Pro Ile Gly Thr Glu Lys Glu Pro
Asn Asn Ser Lys Glu Thr Ala Ser Gly Pro Ile Val
Pro Gly Ile Pro Val Ser Gly Thr Ile Glu Asn Thr
Ser Asp Gln Asp Tyr Phe Tyr Phe Asp Val Ile Thr
Pro Gly Glu Val Lys Ile Asp Ile Asn Lys Leu Gly
Tyr Gly Gly Ala Thr Trp Val Val Tyr Asp Glu Asn
Asn Asn Ala Val Ser Tyr Ala Thr Asp Asp Gly Gln
Asn Leu Ser Gly Lys Phe Lys Ala Asp Lys Pro Gly
Arg Tyr Tyr Ile His Leu Tyr Met Phe Asn Gly Ser
Tyr Met Pro Tyr Arg Ile Asn Ile Glu Gly Ser Val
Gly Arg
(SEQ ID NO: 55)
Glu Ile Lys Asp Leu Ser Glu Asn Lys Leu Pro Val
Ile Tyr Met His Val Pro Lys Ser Gly Ala Leu Asn
Gln Lys Val Val Phe Tyr Gly Lys Gly Thr Tyr Asp
Pro Asp Gly Ser Ile Ala Gly Tyr Gln Trp Asp Phe
Gly Asp Gly Ser Asp Phe Ser Ser Glu Gln Asn Pro
Ser His Val Tyr Thr Lys Lys Gly Glu Tyr Thr Val
Thr Leu Arg Val Met Asp Ser Ser Gly Gln Met Ser
Glu Lys Thr Met Lys Ile Lys Ile Thr Asp Pro Val
Tyr Pro Ile Gly Thr Glu Lys Glu Pro Asn Asn Ser
Lys Glu Thr Ala Ser Gly Pro Ile Val Pro Gly Ile
Pro Val Ser Gly Thr Ile Glu Asn Thr Ser Asp Gln
Asp Tyr Phe Tyr Phe Asp Val Ile Thr Pro Gly Glu
Val Lys Ile Asp Ile Asn Lys Leu Gly Tyr Gly Gly
Ala Thr Trp Val Val Tyr Asp Glu Asn Asn Asn Ala
Val Ser Tyr Ala Thr Asp Asp Gly Gln Asn Leu Ser
Gly Lys Phe Lys Ala Asp Lys Pro Gly Arg Tyr Tyr
Ile His Leu Tyr Met Phe Asn Gly Ser Tyr Met Pro
Tyr Arg Ile Asn Ile Glu Gly Ser Val Gly Arg

Collagen-binding peptides useful in conjunction with the conjugates and methods described herein also include those having at least 85% sequence identity (e.g., at least 85%, 90%, 95%, 97%, 99%, or greater) to one of the foregoing sequences and/or having one or more conservative amino acid substitutions with respect to one of these sequences.

Additionally, collagen-binding peptides derived from human glycoprotein VI (GPVI) have been described, for instance, in U.S. Pat. No. 8,084,577, the disclosure of which is incorporated herein by reference in its entirety. Collagen-binding domains of GPVI can be incorporated into conjugates described herein, for instance, using the synthetic chemistry or protein expression methodologies described below. The sequence of the collagen-binding domain of GPVI is described below:

(SEQ ID NO: 56)
Gln Ser Gly Pro Leu Pro Lys Pro Ser Leu Gln Ala
Leu Pro Ser Ser Leu Val Pro Leu Glu Lys Pro Val
Thr Leu Arg Cys Gln Gly Pro Pro Gly Val Asp Leu
Tyr Arg Leu Glu Lys Leu Ser Ser Ser Arg Tyr Gln
Asp Gln Ala Val Leu Phe Ile Pro Ala Met Lys Arg
Ser Leu Ala Gly Arg Tyr Arg Cys Ser Tyr Gln Asn
Gly Ser Leu Trp Ser Leu Pro Ser Asp Gln Leu Glu
Leu Val Ala Thr Gly Val Phe Ala Lys Pro Ser Leu
Ser Ala Gln Pro Gly Pro Ala Val Ser Ser Gly Gly
Asp Val Thr Leu Gln Cys Gln Thr Arg Tyr Gly Phe
Asp Gln Phe Ala Leu Tyr Lys Glu Gly Asp Pro Ala
Pro Tyr Lys Asn Pro Glu Arg Trp Tyr Arg Ala Ser
Phe Pro Ile Ile Thr Val Thr Ala Ala His Ser Gly
Thr Tyr Arg Cys Tyr Ser Phe Ser Ser Arg Asp Pro
Tyr Leu Trp Ser Ala Pro Ser Asp Pro Leu Glu Leu
Val Val Thr

Collagen-binding peptides useful in conjunction with the conjugates and methods described herein also include those having at least 85% sequence identity (e.g., at least 85%, 90%, 95%, 97%, 99%, or greater) to the foregoing GPVI-derived sequence and/or having one or more conservative amino acid substitutions with respect to this sequence.

Additionally, collagen-binding peptides derived from human fibronectin can be incorporated into the conjugates described herein (e.g., peptides of about 340 residues corresponding to the amino acid sequence between and including Ala260 and Trp599 of human fibronectin) have been described in detail in WO 2000/049159, the disclosure of which is incorporated herein by reference in its entirety.

Collagen-binding peptides useful in conjunction with the conjugates and methods described herein also include those having at least 85% sequence identity (e.g., at least 85%, 90%, 95%, 97%, 99%, or greater) to the foregoing fibronectin-derived sequence and/or having one or more conservative amino acid substitutions with respect to this sequence.

Collagen-binding peptides derived from bone sialoprotein can be incorporated into the conjugates described herein. Such peptide have been described in detail in WO 2005/082941, the disclosure of which is incorporated herein by reference in its entirety. Exemplary sequences derived from the N-terminal domain of bone sialoprotein that bind collagen are summarized below:

(SEQ ID NO: 57)
NGVFKYRPRYFLYKHAYFYPPLKRFPVQ
(SEQ ID NO: 58)
NGVFKYRPRYFLYK
(SEQ ID NO: 59)
HAYFYPPLKRFPVQ

Collagen-binding peptides useful in conjunction with the conjugates and methods described herein also include those having at least 85% sequence identity (e.g., at least 85%, 90%, 95%, 97%, 99%, or greater) to one of the foregoing sequences and/or having one or more conservative amino acid substitutions with respect to these sequences.

In addition to the above, hydroxyapatite-binding domains that can be incorporated into conjugates described herein have been identified, for instance, using phage display techniques. Such peptides are described, for example, in U.S. Pat. No. 8,022,040, the disclosure of which is incorporated herein by reference in its entirety. Exemplary hydroxyapatite-binding domains described therein are summarized in Table 8, below.

TABLE 8
Exemplary hydroxyapatite-binding peptides

| SEQ ID NO. | Amino acid sequence |
|---|---|
| 60 | RPHTITN |
| 61 | QSSYNPI |
| 62 | QTHARHQ |
| 63 | ETRTQLL |
| 64 | HHQRSPA |
| 65 | LQKSPSL |
| 66 | PPKDSRG |
| 67 | SAKKVFS |
| 68 | SQHSTQD |
| 69 | TIHSKPA |
| 70 | TKDWLPS |
| 71 | ANPPLSL |
| 72 | AKQTVPV |
| 73 | ATFSPPL |
| 74 | DQYWGLR |
| 75 | EPNHTRF |
| 76 | HMLAQTF |
| 77 | IGYPVLP |
| 78 | KLSAWSF |
| 79 | MYPLPAP |
| 80 | FTLPTIR |
| 81 | SMAAKSS |
| 82 | SMYDTHS |
| 83 | STLASMR |
| 84 | TLMTTPP |
| 85 | WLPPRTQ |
| 86 | RTPLQPLEDFRP |
| 87 | NTTTDIPSPSQF |
| 88 | TLDKYTRLLSRY |
| 89 | YPIMSHTCCHGV |
| 90 | YEPAAAE |
| 91 | ANPYHRH |
| 92 | ASGPTNV |
| 93 | QNYLLPK |
| 94 | GTQTPQP |
| 95 | HSTGPTR |
| 96 | LSKNPLL |
| 97 | LSKNPLL |
| 98 | KLHASLA |
| 99 | PLTQPSH |
| 100 | PHNPGKL |
| 101 | PTTMTRW |
| 102 | VHLTHGQ |
| 103 | TLAPTFR |
| 104 | VHPRPSL |
| 105 | TLLRTQV |
| 106 | SSPPRVY |
| 107 | SSVPGRP |
| 108 | LPFQPPI |
| 109 | IQHQAKT |
| 110 | LPRDLHATPQQI |
| 111 | LTPTMFNMHGVL |
| 112 | SIPKMIPTESLL |
| 113 | SFQSMSLMTLVV |
| 114 | TQTWPQSSSHGL |
| 115 | YELQMP |
| 116 | AMSQTMTAAIEK |
| 117 | GSAGLKYPLYKS |
| 118 | INFQFLKPSTTR |
| 119 | RHTLPLH |
| 120 | NFAMNLR |
| 121 | NFAMNLR |
| 122 | NPQMQRS |
| 123 | NPQMQRS |
| 124 | NPQMQRS |
| 125 | NYPTLKS |
| 126 | NYPTLKS |
| 127 | QNPRQIY |
| 128 | QNPRQIY |
| 129 | QNPRQIY |
| 130 | QNPRQIY |
| 131 | ETYARPL |
| 132 | ETVCASS |
| 133 | KPMQFVH |
| 134 | KPMQFVH |
| 135 | PAKQKAH |
| 136 | PTTWGHL |
| 137 | PTTWGHL |
| 138 | SASGTPS |
| 139 | SSYEYHA |
| 140 | SSYEYHA |
| 141 | STQAHPW |
| 142 | TVLGTFP |
| 143 | WYPNHLA |
| 144 | TTYNSPP |
| 145 | MTSQTLR |
| 146 | WPANKLSTKSMY |
| 147 | WPANKLSTKSMY |
| 148 | NPYHPTIPQSVH |
| 149 | DKLHRLA |
| 150 | QPGLWPS |
| 151 | ESLKSIS |
| 152 | GSCPPKK |
| 153 | GSLFKAL |
| 154 | HQWDHKY |
| 155 | LSAPMEY |
| 156 | MKVHERS |
| 157 | FVNLLGQ |
| 158 | PIDAFFD |
| 159 | PPNMARA |
| 160 | PTNKPHT |
| 161 | SPNNTRE |
| 162 | SPEMKPR |
| 163 | SSSMAKM |
| 164 | TDHPPKA |
| 165 | TLAFQTA |
| 166 | APLSLSL |
| 167 | HYPTVNF |
| 168 | QHNFRGASSSAP |
| 169 | HQFPXSNLVWKP |
| 170 | LSLRASAATDFQ |
| 171 | MQFTPAPSPSDH |
| 172 | SVFLPTRHSPDL |
| 173 | SVSVGMKPSPRP |
| 174 | SVSVGMKPSPRP |
| 175 | SVSVGMKPSPRP |
| 176 | SVSVGMKPSPRP |
| 177 | SVSVGMNAESA |
| 178 | RHTLPLH |
| 179 | NPQMQRS |
| 180 | NYPTLKS |
| 181 | NYPTLKS |
| 182 | DMRQQRS |
| 183 | QNPRQIY |
| 184 | QNPRQIY |
| 185 | QNPRQIY |
| 186 | QNPRQIY |
| 187 | QNPRQIY |
| 188 | QNPRQIY |
| 189 | QNPRQIY |
| 190 | QNPRQIY |
| 191 | QNPRQIY |
| 192 | QTHSSLW |
| 193 | ETYQQPL |
| 194 | ETYARPL |
| 195 | GTSRLFS |
| 196 | LTQTLQY |
| 197 | KAFDKHG |
| 198 | RPMQFVH |
| 199 | KPMQFVH |
| 200 | KPMQFIH |
| 201 | PAKQKAH |
| 202 | SASGTPS |
| 203 | SSHHHRH |
| 204 | SSYEYHA |
| 205 | TGPTSLS |
| 206 | LRAFPSLPHTVT |
| 207 | NPRSQAT |
| 208 | HRLGHMS |
| 209 | LLPLKFK |
| 210 | LPSIHNL |
| 211 | KATITGM |
| 212 | PDIPLSR |
| 213 | PSMKHWR |
| 214 | SAKGRAD |
| 215 | SRTGAHH |
| 216 | SKTSSTS |
| 217 | SPNNPRE |
| 218 | TLQRMGQ |
| 219 | TMTNMAK |
| 220 | TTLSPRT |
| 221 | TTKNFNK |
| 222 | YPKALRN |
| 223 | VVKSNGE |
| 224 | ITGAY |
| 225 | LPLTPLP |
| 226 | HSMPHMGTYLLT |
| 227 | MQFTPAPSPSDH |
| 228 | MPQTLVLPRSLL |
| 229 | SSTQVQHTLLQT |
| 230 | SWPLYSRDSGLG |
| 231 | SVSVGTEAESXA |
| 232 | SVSVGMKPSPRP |
| 233 | SVSVGMKPSPRP |
| 234 | SVSVGMKPSPRP |
| 235 | SVSVGMNAESYG |
| 236 | THPVVFEDERLF |
| 237 | TLPSPLALLTVH |
| 238 | WPTYLNPSSLKA |
| 239 | ASHNPKL |
| 240 | PAKQKAH |
| 241 | PAKQKAH |
| 242 | SASGTPS |
| 243 | TRFYDSL |
| 244 | QNPRQIY |
| 245 | QNPRQIY |
| 246 | QNPRQIY |
| 247 | QNPRQIY |
| 248 | TGPTSLS |
| 249 | TGPTSLS |
| 250 | NPQMQRS |
| 251 | NPQMQRS |
| 252 | NPQMQRS |
| 253 | NPQMQRS |
| 254 | NPQMQRS |
| 255 | KPMQFVH |
| 256 | SSYEYHA |
| 257 | STQAHPW |
| 258 | GTSRLFS |
| 259 | NYPTLKS |
| 260 | NYPTLKS |
| 261 | NYPTLKS |
| 262 | NYPTLKS |
| 263 | NYPTLKS |
| 264 | NYPTLKS |
| 265 | NYPTLKS |
| 266 | HAPVQPN |
| 267 | NPYHPTIPQSVH |
| 268 | NPYHPTIPQSVH |
| 269 | NPYHPTIPQSVH |
| 270 | NPYHPTIPQSVH |
| 271 | HQFISPEPFLIS |
| 272 | SPNFSWLPLGTTSPNFS |
| 273 | WLPLGTT |
| 274 | SVSVGMKPSPRP |
| 275 | SVSVGMKPSPRP |
| 276 | TPLTSPSLVRPQ |
| 277 | TPLSYLKGLVTV |
| 278 | NPMIMNQ |
| 279 | NPMIMNQ |
| 280 | NITQLGS |
| 281 | HTLLSTT |
| 282 | HTLLSTT |
| 283 | HTLLSTT |
| 284 | LGPGKAF |
| 285 | LGPGKAF |
| 286 | LGPGKAF |
| 287 | LGPGKAF |
| 288 | KTSSWAN |
| 289 | KMNHMPN |
| 290 | SLLTPWL |
| 291 | TLGLPML |
| 292 | TGLAKT |
| 293 | IRLIS |
| 294 | LGPGKAF |
| 295 | LGPGKAF |
| 296 | LGPGKAF |
| 297 | DLNYFTLSSKRE |
| 298 | DLNYFTLSSKRE |
| 299 | TMGFTAPRFPHY |
| 300 | TMGFTAPRFPHY |
| 301 | TMGFTAPRFPHY |
| 302 | TMGFTAPRFPHY |
| 303 | HTLLSTT |
| 304 | HTLLSTT |
| 305 | LASTTHV |
| 306 | LGPGKAF |
| 307 | LGPGKAF |
| 308 | LGPGKAF |
| 309 | SLLTPWL |
| 310 | NERQMEL |
| 311 | NKPLSTL |
| 312 | HTLLSTT |
| 313 | LKPFSGA |
| 314 | LGPGKAF |
| 315 | LGPGKAF |
| 316 | LGPGKAF |
| 317 | LGPGKAF |
| 318 | STSAKHW |
| 319 | TMGFTAPRFPHY |
| 320 | TMGFTAPRFPHY |
| 321 | TMGFTAPRFPHY |
| 322 | TMGFTAPRFPHY |
| 323 | TMGFTAPRFPHY |
| 324 | TMGFTAPRFPHY |
| 325 | TMGFTAPRFPHY |
| 326 | CNYPTLKSC |

Hydroxyapatite-binding peptides useful in conjunction with the conjugates and methods described herein also include those having at least 85% sequence identity (e.g., at least 85%, 90%, 95%, 97%, 99%, or greater) to one of the foregoing sequences and/or having one or more conservative amino acid substitutions with respect to these sequences.

Exemplary targeting moieties that can be used to localize a TGF-β antagonist to hydroxyapatite, and thus ossesous tissue, include polyanionic peptides, such as those that contain one or more amino acids bearing a side-chain substituent selected from the group consisting of carboxylate, sulfonate, phosphonate, and phosphate. For instance, hydroxyapatite-binding targeting moieties include those that feature a plurality of consecutive or discontinuous aspartate or glutamate residues. Polyanionic peptides can bind hydroxyapatite by virtue, for instance, of electrostatic interactions between negatively charged substituents within the peptide, such as one or more carboxylate, sulfonate, phosphonate, or phosphate substituents, among others, to positively charged calcium ions present within hydroxyapatite.

In some embodiments, the polyanionic peptide contains (e.g., consists of) one or more glutamate residues (e.g., 1-25 glutamate residues, or more, such as 1, 2, 3, 4, 5, 6, 7, 8, 9, 10, 11, 12, 13, 14, 15, 16, 17, 18, 19, 20, 21, 22, 23, 24, or 25, or more, glutamate residues). In some embodiments, the polyanionic peptide contains (e.g., consists of) from 3 to 20 glutamate residues (e.g., 3, 4, 5, 6, 7, 8, 9, 10, 11, 12, 13, 14, 15, 16, 17, 18, 19, or 20 glutamate residues). In some embodiments, the polyanionic peptide contains (e.g., consists of) from 5 to 15 glutamate residues (e.g., 5, 6, 7, 8, 9, 10, 11, 12, 13, 14, or 15 glutamate residues). In some embodiments, the polyanionic peptide contains (e.g., consists of) from 8 to 12 glutamate residues (e.g., 8, 9, 10, 11, or 12 glutamate residues). In some embodiments, the polyanionic peptide contains (e.g., consists of) 5 glutamate residues. In some embodiments, the polyanionic peptide contains (e.g., consists of) 6 glutamate residues. In some embodiments, the polyanionic peptide contains (e.g., consists of) 7 glutamate residues. In some embodiments, the polyanionic peptide contains (e.g., consists of) 8 glutamate residues. In some embodiments, the polyanionic peptide contains (e.g., consists of) 9 glutamate residues. In some embodiments, the polyanionic peptide contains (e.g., consists of) 10 glutamate residues. In some embodiments, the polyanionic peptide contains (e.g., consists of) 11 glutamate residues. In some embodiments, the polyanionic peptide contains (e.g., consists of) 12 glutamate residues. In some embodiments, the polyanionic peptide contains (e.g., consists of) 13 glutamate residues. In some embodiments, the polyanionic peptide contains (e.g., consists of) 14 glutamate residues. In some embodiments, the polyanionic peptide contains (e.g., consists of) 15 glutamate residues.

The polyanionic peptide may be a peptide of the formula Eₙ, wherein E designates a glutamate residue and n is an integer from 1 to 25. For instance, the polyanionic peptide may be of the formula E₁, E₂, E₃, E₄, E₅, E₆, E₇, E₈, E₉, E₁₀, E₁₁, E₁₂, E₁₃, E₁₄, E₁₅, E₁₆, E₁₇, E₁₈, E₁₉, E₂₀, E₂₁, E₂₂, E₂₃, E₂₄, or E₂₅. In some embodiments, the peptide is a peptide of the formula XₙEₘXₒEₚ, wherein E designates a glutamate residue, each X independently designates any naturally-occurring amino acid, m represents an integer from 1 to 25, and n and o each independently represent integers from 0 to 5, and p represents an integer from 1 to 10.

In some embodiments, the polyanionic peptide contains (e.g., consists of) one or more aspartate residues (e.g., 1-25 aspartate residues, or more, such as 1, 2, 3, 4, 5, 6, 7, 8, 9, 10, 11, 12, 13, 14, 15, 16, 17, 18, 19, 20, 21, 22, 23, 24, or 25, or more, aspartate residues). In some embodiments, the polyanionic peptide contains (e.g., consists of) from 3 to 20 aspartate residues (e.g., 3, 4, 5, 6, 7, 8, 9, 10, 11, 12, 13, 14, 15, 16, 17, 18, 19, or 20 aspartate residues). In some embodiments, the polyanionic peptide contains (e.g., consists of) from 5 to 15 aspartate residues (e.g., 5, 6, 7, 8, 9, 10, 11, 12, 13, 14, or 15 aspartate residues). In some embodiments, the polyanionic peptide contains (e.g., consists of) from 8 to 12 aspartate residues (e.g., 8, 9, 10, 11, or 12 aspartate residues). In some embodiments, the polyanionic peptide contains (e.g., consists of) 5 aspartate residues. In some embodiments, the polyanionic peptide contains (e.g., consists of) 6 aspartate residues. In some embodiments, the polyanionic peptide contains (e.g., consists of) 7 aspartate residues. In some embodiments, the polyanionic peptide contains (e.g., consists of) 8 aspartate residues. In some embodiments, the polyanionic peptide contains (e.g., consists of) 9 aspartate residues. In some embodiments, the polyanionic peptide contains (e.g., consists of) 10 aspartate residues. In some embodiments, the polyanionic peptide contains (e.g., consists of) 11 aspartate residues. In some embodiments, the polyanionic peptide contains (e.g., consists of) 12 aspartate residues. In some embodiments, the polyanionic peptide contains (e.g., consists of) 13 aspartate residues. In some embodiments, the polyanionic peptide contains (e.g., consists of) 14 aspartate residues. In some embodiments, the polyanionic peptide contains (e.g., consists of) 15 aspartate residues.

The polyanionic peptide may be a peptide of the formula Dₙ, wherein D designates an aspartate residue and n is an integer from 1 to 25. For instance, the polyanionic peptide may be of the formula D₁, D₂, D₃, D₄, D₅, D₆, D₇, D₈, D₉, D₁₀, D₁₁, D₁₂, D₁₃, D₁₄, D₁₅, D₁₆, D₁₇, D₁₈, D₁₉, D₂₀, D₂₁, D₂₂, D₂₃, D₂₄, or D₂₅. In some embodiments, the peptide is a peptide of the formula XₙDₘXₒDₚ, wherein D designates an aspartate residue, each X independently designates any naturally-occurring amino acid, m represents an integer from 1 to 25, and n and o each independently represent integers from 0 to 5, and p represents an integer from 1 to 10.

In some embodiments, the aspartate residues are consecutive. In some embodiments, the aspartate residues are discontinuous.

In some embodiments, the ratio of amino acids bearing a side-chain that is negatively-charged at physiological pH to the total quantity of amino acids in the polyanionic peptide is from about 0.5 to about 2.0.

### Peptide Synthesis Techniques

Systems and processes for performing solid phase peptide synthesis of conjugates described herein include those that are known in the art and have been described, for instance, in U.S. Pat. Nos. 9,169,287; 9,388,212; 9,206,222; 6,028,172; and 5,233,044, among others, the disclosures of each of which are incorporated herein by reference as they pertain to protocols and techniques for the synthesis of peptides on solid support. Solid phase peptide synthesis is a known process in which amino acid residues are added to peptides that have been immobilized on a solid support, such as a polymeric resin (e.g., a hydrophilic resin, such as a polyethylene-glycol-containing resin, or hydrophobic resin, such as a polystyrene-based resin).

Peptides, such as those containing protecting groups at amino, hydroxy, thiol, and carboxy substituents, among others, may be bound to a solid support such that the peptide is effectively immobilized on the solid support. For example, the peptides may be bound to the solid support via their C termini, thereby immobilizing the peptides for subsequent reaction in at a resin-liquid interface.

The process of adding amino acid residues to immobilized peptides can include exposing a deprotection reagent to the immobilized peptides to remove at least a portion of the protection groups from at least a portion of the immobilized peptides. The deprotection reagent exposure step can be configured, e.g., such that side-chain protection groups are preserved, while N-termini protection groups are removed. For instance, an exemplary amino protecting may contain fluorenylmethyloxycarbonyl (Fmoc). A deprotection reagent containing piperidine (e.g., a piperidine solution in an appropriate organic solvent, such as dimethyl formamide (DMF)) may be exposed to the immobilized peptides such that the Fmoc protecting groups are removed from at least a portion of the immobilized peptides. Other protecting groups suitable for the protection of amino substituents include, for instance, the tert-butyloxycarbonyl (Boc) moiety. A deprotection reagent comprising a strong acid, such as trifluoroacetic acid (TFA) may be exposed to immobilized peptides containing a Boc-protected amino substituent so as to remove the Boc protecting group by an ionization process. In this way, peptides can be protected and deprotected at specific sites, such as at one or more side-chains or at the N- or C-terminus of an immobilized peptide so as to append chemical functionality regioselectively at one or more of these positions. This can be used, for instance, to derivative a side-chain of an immobilized peptide, or to synthesize a peptide, e.g., from the C-terminus to the N-terminus.

The process of adding amino acid residues to immobilized peptides can include, for instance, exposing protected, activated amino acids to the immobilized peptides such that at least a portion of the activated amino acids are bonded to the immobilized peptides to form newly-bonded amino acid residues. For example, the peptides may be exposed to activated amino acids that react with the deprotected N-termini of the peptides so as to elongate the peptide chain by one amino acid. Amino acids can be activated for reaction with the deprotected peptides by reaction of the amino acid with an agent that enhances the electrophilicity of the carbonyl carbon of the amino acid. For example, phosphonium and uronium salts can, in the presence of a tertiary base (e.g., diisopropylethylamine (DIPEA) and triethylamine (TEA), among others), convert protected amino acids into activated species (for example, BOP, PyBOP, HBTU, and TBTU all generate HOBt esters). Other reagents can be used to help prevent racemization that may be induced in the presence of a base. These reagents include carbodiimides (for example, DCC or WSCDI) with an added auxiliary nucleophile (for example, 1-hydroxy-benzotriazole (HOBt), 1-hydroxy-azabenzotriazole (HOAt), or HOSu) or derivatives thereof. Another reagent that can be utilized to prevent racemization is TBTU. The mixed anhydride method, using isobutyl chloroformate, with or without an added auxiliary nucleophile, can also be used, as well as the azide method, due to the low racemization associated with this reagent. These types of compounds can also increase the rate of carbodiimide-mediated couplings, as well as prevent dehydration of Asn and Gln residues. Typical additional reagents include also bases such as N,N-diisopropylethylamine (DIPEA), triethylamine (TEA) or N-methylmorpholine (NMM). These reagents are described in detail, for instance, in U.S. Pat. No. 8,546,350, the disclosure of which is incorporated herein in its entirety.

Cyclic peptides can be synthesized using solid-phase peptide synthesis techniques. For instance, a side-chain substituent, such as an amino, carboxy, hydroxy, or thiol moiety can be covalently bound to a resin, leaving the N-terminus and C-terminus of the amino acid exposed in solution. The N- or C-terminus can be chemically protected, for instance, while reactions are carried out that elongate the peptide chain. The termini of the peptide can then be selectively deprotected and coupled to one another while the peptide is immobilized by way of the side-chain linkage to the resin. Techniques and reagents for the synthesis of head-to-tail cyclic peptides are known in the art and are described, for instance, in U.S. Pat. Nos. 9,388,212 and 7,589,170, the disclosures of which are incorporated herein by reference in their entirety.

### Linkers for Conjugate Synthesis

### Synthetic Linkers

A variety of linkers can be used to covalently couple reactive residues within peptidic a TGF-β antagonist and a bone-targeting moiety to one another, for instance, so as to form a conjugate as described herein. Exemplary linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for chemical coupling). Examples of linkers useful for the synthesis of conjugates described herein include those that contain electrophiles, such as Michael acceptors (e.g., maleimides), activated esters, electron-deficient carbonyl compounds, and aldehydes, among others, suitable for reaction with nucleophilic substituents present within antibodies, antigen-binding fragments, proteins, peptides, and small molecules, such as amine and thiol moieties. For instance, linkers suitable for the synthesis of therapeutic conjugates include, without limitation, alkyl, cycloalkyl, and heterocycloalkyl linkers, such as open-chain ethyl, propyl, butyl, hexyl, heptyl, octyl, nonyl, or decyl chains, cyclohexyl groups, cyclopentyl groups, cyclobutyl groups, cyclopropyl groups, piperidinyl groups, morpholino groups, or others containing two reactive moieties (e.g., halogen atoms, aldehyde groups, ester groups, acyl chloride groups, acyl anhydride groups, tosyl groups, mesyl groups, or brosyl groups, among others, that can be displaced by reactive nucleophilic atoms present within a TGF-β antagonist peptide and/or bone-targeting moiety), aryl or heteroaryl linkers, such as benzyl, napthyl, or pyridyl groups containing two halomethyl groups that can be displaced by reactive nucleophilic atoms present within a TGF-β antagonist peptide and/or bone-targeting moiety. Exemplary linkers include succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, for instance, Liu et al., 18:690-697, 1979, the disclosure of which is incorporated herein by reference as it pertains to linkers for chemical conjugation. Additional linkers include the non-cleavable maleimidocaproyl linkers, which are described by Doronina et al., Bioconjugate Chem. 17:14-24, 2006, the disclosure of which is incorporated herein by reference as it pertains to linkers for chemical conjugation.

Additional linkers through which one component of a conjugate may be bound to another as described herein include linkers that are covalently bound to one component of the conjugate (e.g., a TGF-β antagonist, such as an antibody, protein, peptide, or small molecule) on one end of the linker and, on the other end of the linker, contain a chemical moiety formed from a coupling reaction between a reactive substituent present on the linker and a reactive substituent present within the other component of the conjugate (e.g., bone-targeting moiety described herein). Exemplary reactive substituents that may be present within a component of the conjugate include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. Linkers useful in conjunction with the conjugates described herein include, without limitation, linkers containing chemical moieties formed by coupling reactions as depicted in Table 9 below. Curved lines designate points of attachment to each component of the conjugate.

TABLE 9
Exemplary chemical moieties formed by coupling reactions in the formation of TGF-β antagonist
conjugates

| Exemplary | |
|---|---|
| Coupling Reactions | Chemical Moiety Formed by Coupling Reactions |
| [3 + 2] Cycloaddition | 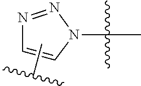 |
| [3 + 2] Cycloaddition | 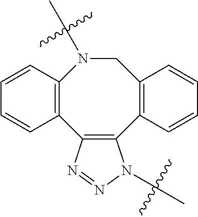 [SMILES: *N1Cc2ccccc2-c2c(nnn2*)-c2ccccc21] |
| [3 + 2] Cycloaddition, Esterification | 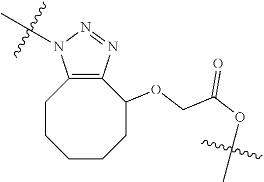 [SMILES: *OC(=O)COC1CCCCCc2c1nnn2*] |
| [3 + 2] Cycloaddition, Esterification | 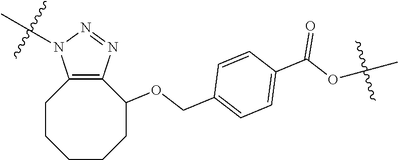 [SMILES: *OC(=O)c1ccc(COC2CCCCCc3c2nnn3*)cc1] |
| [3 + 2] Cycloaddition, Esterification | 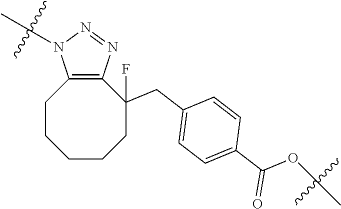 [SMILES: *OC(=O)c1ccc(CC2(F)CCCCCc3c2nnn3*)cc1] |
| [3 + 2] Cycloaddition, Esterification | 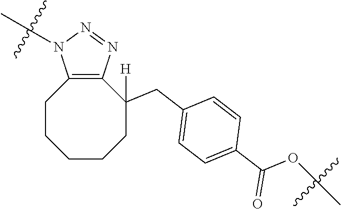 [SMILES: *OC(=O)c1ccc(CC2CCCCCc3c2nnn3*)cc1] |
| [3 + 2] Cycloaddition, Esterification | 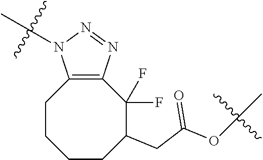 [SMILES: *OC(=O)CC1CCCCc2c(nnn2*)C1(F)F] |
| [3 + 2] Cycloaddition, Esterification | 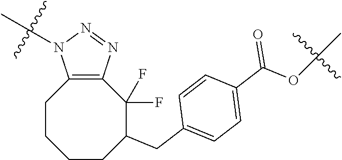 [SMILES: *OC(=O)c1ccc(CC2CCCCc3c(nnn3*)C2(F)F)cc1] |
| [3 + 2] Cycloaddition, Esterification | 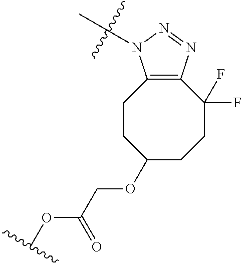 [SMILES: *OC(=O)COC1CCc2c(nnn2*)C(F)(F)CC1] |
| [3 + 2] Cycloaddition, Esterification | 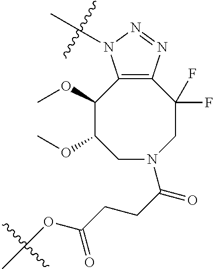 [SMILES: *OC(=O)CCC(=O)N1C[C@H](OC)[C@@H](OC)c2c(nnn2*)C(F)(F)C1] |
| [3 + 2] Cycloaddition, Esterification | 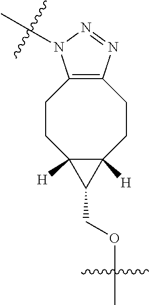 [SMILES: *OC[C@@H]1[C@@H]2CCc3c(nnn3*)CC[C@@H]21] |
| [3 + 2] Cycloaddition, Esterification | 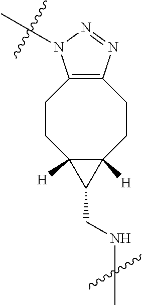 [SMILES: *NC[C@@H]1[C@@H]2CCc3c(nnn3*)CC[C@@H]21] |
| [3 + 2] Cycloaddition, Esterification | 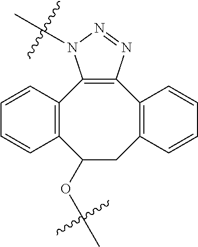 [SMILES: *OC1Cc2ccccc2-c2nnn(*)c2-c2ccccc21] |
| [3 + 2] Cycloaddition, Etherification | 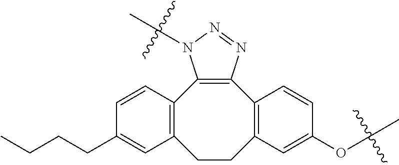 [SMILES: *Oc1ccc2c(c1)CCc1cc(CCCC)ccc1-c1c-2nnn1*] |
| [3 + 2] Cycloaddition | 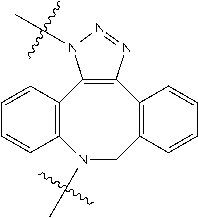 [SMILES: *N1Cc2ccccc2-c2nnn(*)c2-c2ccccc21] |
| Michael addition | 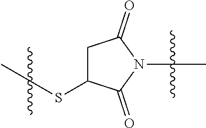 [SMILES: *SC1CC(=O)N(*)C1=O] |
| Michael addition | 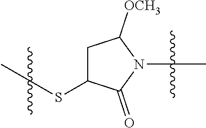 [SMILES: *SC1CC(OC)N(*)C1=O] |
| Imine condensation, Amidation | 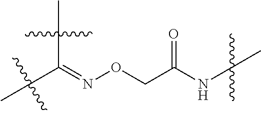 [SMILES: *NC(=O)CON=C(*)*] |
| Imine condensation | 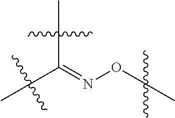 [SMILES: *ON=C(*)*] |
| Disulfide formation | 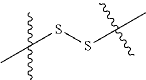 [SMILES: *SS*] |
| Thiol alkylation | 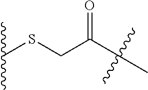 [SMILES: *SCC(*)=O] |
| Condensation, Michael addition | 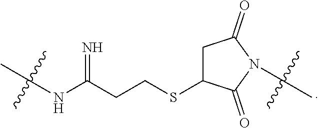 [SMILES: *NC(=N)CCSC1CC(=O)N(*)C1=O] |

### Peptidic Linkers

In addition to the synthetic linkers described above, the binding of one component of a conjugate to another as described herein can be effectuated by way of a peptide linker. TGF-β antagonists and conjugates thereof composed of proteinogenic amino acids in which one or more components are joined by a peptide linker can be prepared, for instance, by expressing a nucleic acid encoding the linker in combination with the components of the conjugate. Exemplary peptide linkers include those that contain one or more glycine residues. Such linkers may be sterically flexible due to the ability of glycine to access a variety of torsional angles. For instance, peptide linkers useful in conjunction with the compositions and methods described herein include polyglycine, such as GGG (SEQ ID NO: 335). Additional examples of peptidic linkers include those that also contain one or more polar amino acids, such as serine or threonine. For instance, linkers useful in conjunction with the compositions and methods described herein include those that contain one or more repeats of the peptide GGGGS (SEQ ID NO: 336). Additional linkers include GGGGSGGGGSGGGGSG (SEQ ID NO: 337), as well as those that contain one or more cationic or anionic residues, such as a lysine, arginine, aspartate, or glutamate residue.

### Methods for the Expression of Conjugates in Host Cells

In addition to synthetic chemistry techniques such as those described above, conjugates described herein (e.g., protein conjugates wherein the TGF-β antagonist is bound to a bone-targeting moiety by one or more peptide bonds) can be expressed in host cells, for instance, by delivering to the host cell a nucleic acid encoding the conjugate protein. The sections that follow describe a variety of established techniques that can be used for the purposes of delivering nucleic acids encoding therapeutic conjugates described herein to a host cell for the purposes of expressing the conjugate protein.

### Transfection Techniques

Techniques that can be used to introduce a polynucleotide, such as nucleic acid encoding a TGF-β antagonist peptide describe herein, into a cell (e.g., a mammalian cell, such as a human cell) are well known in the art. For instance, electroporation can be used to permeabilize mammalian cells (e.g., human cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of cells of interest include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human cell. Squeeze-poration is described in detail, e.g., in Sharei et al., Journal of Visualized Experiments 81:e50980 (2013), the disclosure of which is incorporated herein by reference.

Lipofection represents another technique useful for transfection of cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for instance, by direct fusion of the liposome with the cell membrane or by endocytosis of the complex. Lipofection is described in detail, for instance, in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane include activated dendrimers (described, e.g., in Dennig, Topics in Current Chemistry 228:227 (2003), the disclosure of which is incorporated herein by reference) and diethylaminoethyl (DEAE)-dextran, the use of which as a transfection agent is described in detail, for instance, in Gulick et al., Current Protocols in Molecular Biology 40:1:9.2:9.2.1 (1997), the disclosure of which is incorporated herein by reference. Magnetic beads are another tool that can be used to transfect cells in a mild and efficient manner, as this methodology utilizes an applied magnetic field in order to direct the uptake of nucleic acids. This technology is described in detail, for instance, in US 2010/0227406, the disclosure of which is incorporated herein by reference.

Another useful tool for inducing the uptake of exogenous nucleic acids by cells is laserfection, a technique that involves exposing a cell to electromagnetic radiation of a particular wavelength in order to gently permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Incorporation of Genes by Gene Editing Techniques

In addition to the above, a variety of tools have been developed that can be used for the incorporation of exogenous genes, e.g., exogenous genes encoding a TGF-β antagonist peptide or conjugate described herein, into cells, such as a human cell. One such method that can be used for incorporating polynucleotides encoding a TGF-β antagonist or conjugate described herein into cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In some instances, these excision sites may be terminal repeats or inverted terminal repeats. Once excised from the transposon, the gene encoding a TGF-β antagonist peptide or conjugate can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene encoding the TGF-β antagonist peptide or conjugate to the DNA of the mammalian cell genome completes the incorporation process. In some cases, the transposon may be a retrotransposon, such that the gene encoding the TGF-β antagonist peptide or conjugate is first transcribed to an RNA product and then reverse-transcribed to DNA before incorporation in the mammalian cell genome. Exemplary transposon systems include the piggyback transposon (described in detail in, e.g., WO 2010/085699) and the sleeping beauty transposon (described in detail in, e.g., US 2005/0112764), the disclosures of each of which are incorporated herein by reference as they pertain to transposons for use in gene delivery to a cell of interest, such as a mammalian cell (e.g., a human cell).

Another tool for the integration of genes encoding TGF-β antagonist peptides or conjugates described herein into the genome of a cell, such as a human cell, is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a sequence of interest by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a particular sequence and localize the Cas9 nuclease to this site. In this manner, highly site-specific cas9-mediated DNA cleavage can be engendered in a foreign polynucleotide because the interaction that brings cas9 within close proximity of the DNA molecule of interest is governed by RNA:DNA hybridization. As a result, one can theoretically design a CRISPR/Cas system to cleave any DNA molecule of interest. This technique has been exploited in order to edit eukaryotic genomes (Hwang et al., Nature Biotechnology 31:227 (2013)) and can be used as an efficient means of site-specifically editing cell genomes in order to cleave DNA prior to the incorporation of a gene encoding a gene. The use of CRISPR/Cas to modulate gene expression has been described in, for instance, U.S. Pat. No. 8,697,359, the disclosure of which is incorporated herein by reference as it pertains to the use of the CRISPR/Cas system for genome editing. Alternative methods for site-specifically cleaving genomic DNA prior to the incorporation of a gene of interest in a cell include the use of zinc finger nucleases (ZFNs) and transcription activator-like effector nucleases (TALENs). Unlike the CRISPR/Cas system, these enzymes do not contain a guiding polynucleotide to localize to a specific sequence. Sequence specificity is instead controlled by DNA binding domains within these enzymes. The use of ZFNs and TALENs in genome editing applications is described, e.g., in Urnov et al., Nature Reviews Genetics 11:636 (2010); and in Joung et al., Nature Reviews Molecular Cell Biology 14:49 (2013), the disclosure of each of which are incorporated herein by reference as they pertain to compositions and methods for genome editing.

Additional genome editing techniques that can be used to incorporate polynucleotides encoding a conjugate described herein into the genome of a cell of interest, such as a mammalian cell, include the use of ARCUS™ meganucleases that can be rationally designed so as to site-specifically cleave genomic DNA. The use of these enzymes for the incorporation of genes encoding a TGF-β antagonist peptide or conjugate described herein into the genome of a mammalian cell (e.g., a human cell) is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a gene of interest into the nuclear DNA of a cell, such as a mammalian cell (e.g., a human cell). These single-chain nucleases have been described extensively in, for example, U.S. Pat. Nos. 8,021,867 and 8,445,251, the disclosures of each of which are incorporated herein by reference as they pertain to compositions and methods for genome editing.

### Viral Vectors for Nucleic Acid Delivery

Viral genomes provide a rich source of vectors that can be used for the efficient delivery of exogenous genes encoding TGF-β antagonist peptides and conjugates described herein into the genome of a cell (e.g., a mammalian cell, such as a human cell). Viral genomes are particularly useful vectors for gene delivery because the polynucleotides contained within such genomes are typically incorporated into the genome of a cell by generalized or specialized transduction. These processes occur as part of the natural viral replication cycle, and do not require added proteins or reagents in order to induce gene integration. Examples of viral vectors include AAV, retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding TGF-β antagonist peptides described herein to a mammalian cell (e.g., a human cell) include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the disclosure of which is incorporated herein by reference as it pertains to viral vectors for use in gene delivery.

### Methods of Therapeutic Treatment

Conjugates described herein can be administered to a mammalian subject (e.g., a human) suffering from a disease associated with elevated TGF-β activity or elevated bone turnover in order to improve the condition of the patient by attenuating TGF-β signaling specifically at the site of bone tissue. Conjugates of the invention can be administered to a subject, e.g., via any of the routes of administration described herein, such as subcutaneously, intradermally, intramuscularly, intraperitoneally, intravenously, or orally, or by nasal or by epidural administration. Conjugates described herein can be formulated with excipients, biologically acceptable carriers, and may be optionally conjugated to, admixed with, or co-administered separately (e.g., sequentially) with additional therapeutic agents.

Diseases and conditions that can be treated using the conjugates described herein include osteogenesis imperfecta (OI), such as Type I osteogenesis imperfecta, Type II osteogenesis imperfecta, Type III osteogenesis imperfecta, Type IV osteogenesis imperfecta, Type V osteogenesis imperfecta, Type VI osteogenesis imperfecta, Type VII osteogenesis imperfecta, Type VIII osteogenesis imperfecta, Type XI osteogenesis imperfecta, Type X osteogenesis imperfecta, or Type XI osteogenesis imperfecta. These conditions are described, e.g., in Forlino, Nat. Rev. Endo. 7:540 (2011), the disclosure of which is incorporated herein by reference. Osteogenesis imperfecta encompasses a group of congenital bone disorders characterized by deficiencies in one or more proteins involved in bone matrix deposition or homeostasis. Though phenotypes vary among OI types, common symptoms include incomplete ossification of bones and teeth, reduced bone mass, brittle bones, and pathologic fractures. Type-I collagen is one of the most abundant connective tissue proteins in both calcified and non-calcified tissues. Accurate synthesis, post-translational modification, and secretion of type-I collagen are necessary for proper tissue development, maintenance, and repair. Most mutations identified in individuals with 01 result in reduced synthesis of type-I collagen, or incorrect synthesis and/or processing of type-I collagen.

In addition to mutations to the type-I collagen gene, other mutations in genes that participate in the intracellular trafficking and processing of collagens have been identified in OI affected individuals. These genes include molecular chaperones, such as FK506 binding protein 10 (FKBPIO) and heat shock protein 47 (HSP47) (Alanay et al., 2010; Christiansen et al., 2010; Kelley et al., 2011). Additional mutations have been identified in intermolecular collagen cross-linking genes, such as procollagen-lysine, 2-oxoglutarate 5-dioxygenase 2 (PLOD2), and in members of the collagen prolyl hydroxylase family of genes, including leucine proline-enriched proteoglycan (leprecan) (LEPRE1), peptidylprolyl isomerase B (cyclophilin B) (CYPB), and cartilage associated protein (CRTAP) (Morello et al., 2006; Cabral et al., 2007; Baldridge et al., 2008; van Dijk et al., 2009; Choi et al., 2009; Barnes et al., 2010; Pyott et al., 2011). Mutations aside, proteins such as bone morphogenetic protein (BMP) and transforming growth factor β (TGFP) and their respective receptors are thought to participate in the various OI phenotypes, though the exact mechanisms of their actions are unknown (Gebken et al., 2000). In an embodiment, TGF expression is regulated by molecules that bind type-I and type-II collagen. In certain embodiment, a small leucine rich proteoglycan (SLRP) regulates TGF expression. In a specific embodiment, decorin regulates TGFP synthesis. In a certain embodiment, decorin does not bind type-I or type-II collagen in which the 3-hydroxyproline site is absent at position 986 of the type-I and/or type-II collagen molecules.

The vertebrate skeleton is comprised of bone, which is a living, calcified tissue that provides structure, support, protection, and a source of minerals for regulating ion transport. Bone is a specialized connective tissue that is comprised of both cellular and acellular components. The acellular extracellular matrix (ECM) contains both collagenous and non-collagenous proteins, both of which participate in the calcification process. A correctly secreted and aligned ECM is critical for proper bone formation. Pathology results when any of the ECM proteins are absent, malformed or misaligned, as is evidenced in osteogenesis imperfecta.

Under normal homeostatic conditions, osteoblasts and osteoclasts work in unison to maintain bone integrity. Pathology results when bone deposition and bone resorption become uncoupled. For example, osteopetrosis is a bone disease characterized by overly dense, hard bone that is a result of unresorptive osteoclasts, while osteoporosis is a bone disorder characterized by brittle, porous bones which can result from increased osteoclast activity. Elevated TGF-β signaling in osseous tissue can lead to heightened osteoclast activity relative to osteoblast activity, which can in turn lead to osteogenesis imperfecta and promote the aberrant bone resorption associated with this condition. Osteoclasts are thus a useful target for therapeutic intervention. The conjugates described herein can be used to modulate bone resorption by attenuating TGF-β signaling, thereby attenuating osteoclast activity and enhancing osteoblast viability, thereby restoring bone turnover homeostasis.

Several methods can be used to measure and characterize the structure, density, and quality of bone, including histology and histomorphometry, atomic force microscopy, confocal Raman microscopy, nanoindentation, three-point bending test, X-ray imaging, and micro computed tomography (μ-CT). Using these exemplary techniques, for instance, one of skill in the art can monitor the progression of treatment and the effectiveness of therapy. For instance, an improvement in bone integrity, a slowing of bone resorption, and a restoration of homeostasis of bone turnover as determined by one or more of the above methods (or other methods known in the art) can be indicators of effective therapeutic treatment.

Additional disease and conditions that can be treated with the conjugates described herein include, for instance, renal osteodystrophy, hyperparathyroid induced bone disease, diabetic bone disease, osteoarthritis, steroid induced bone disease, disuse osteoporosis, and Cerebral Palsy, McCune-Albright Syndrome, Gaucher Disease, Hyperoxaluria, Paget Disease of bone, and Juvenile Paget Disease, metastatic bone cancer (e.g., wherein the metastasis is a secondary metastasis to breast cancer or prostate cancer), osteoporosis, fibrous dysplasia, Calmurati-Engleman Disease, Marfan's Syndrome, osteoglophonic dysplasia, autosomal dominant osteopetrosis, osteoporosis, osteoporosis-pseudoglioma syndrome, juvenile, geroderma osteodysplastica, osteogenesis imperfecta congenita, microcephaly, cataracts, pseudohypoparathyroidism, Cleidocranial Dysplasia, Dyskeratosis Congenita, Exudative Vitreoretinopathy 1, Schimmelpenning-Feuerstein-Mims Syndrome, Prader-Willi Syndrome, Achondrogenesis, Antley-Bixler Syndrome, Aspartylglucosaminuria, Celiac Disease, Cerebrooculofacioskeletal Syndrome 1, Lysinuric Protein Intolerance, neuropathy, dyskeratosis congenita, Ehlers-Danlos Syndrome, epiphyseal dysplasia, hyaline fibromatosis syndrome, Perrault Syndrome 1, hemochromatosis, homocystinuria (e.g., due to cystathionine beta-synthase deficiency), hypophosphatemic rickets with hypercalciuria, desbuquois dysplasia, multiple pterygium syndrome, lethal congenital contracture syndrome 1, mitochondrial DNA depletion Ssndrome 6 (hepatocerebral Type), Niemann-Pick Disease, osteopetrosis, porphyria, Rothmund-Thomson Syndrome, Wilson Disease, Dent Disease 1, occipital horn syndrome, hyperglycerolemia, hypophosphatemic rickets, Lowe Oculocerebrorenal Syndrome, renal tubulopathy, diabetes mellitus, cerebellar ataxia, vitamin D hydroxylation-deficient rickets, Warburg micro syndrome 1, Stuve-Wiedemann Syndrome, Blue Rubber Bleb Nevus syndrome, Singleton-Merten Syndrome, microcephalic osteodysplastic primordial dwarfism, dysosteosclerosis, Hallermann-Streiff Syndrome, Bruck Syndrome 1, multiple pterygium syndrome (e.g., X-Linked), spondylometaphyseal dysplasia with dentinogenesis imperfecta, Hall-Riggs Mental Retardation Syndrome, infantile multisystem neurologic disease with osseous fragility, acrocephalopolysyndactyly Type III, acroosteolysis, ACTH-independent macronodular adrenal hyperplasia, amino aciduria with mental deficiency, arthropathy, bone fragility (e.g., with craniosynostosis, ocular proptosis, hydrocephalus, and distinctive facial features), brittle cornea syndrome, cerebrotendinous xanthomatosis, Cri-Du-Chat Syndrome, dysplasia epiphysealis hemimelica, autosomal dominant Ehlers-Danlos Syndrome, familial osteodysplasia, Flynn-Aird Syndrome, geroderma osteodysplastica, glycogen storage disease Ia, Hutchinson-Gilford Progeria Syndrome, Infantile Systemic Hyalinosis, hypertrichotic osteochondrodysplasia, hyperzincemia with functional zinc depletion, hypophosphatasia, autosomal dominant hypophosphatemic rickets, X-linked recessive hypophosphatemic rickets, Lichtenstein Syndrome, macroepiphyseal dysplasia (e.g., with osteoporosis wrinkled skin, and agedappearance), Menkes Disease, Mental Retardation (e.g., X-Linked, Snyder-Robinson type), Jansen type metaphyseal chondrodysplasia, microspherophakia-metaphyseal dysplasia, morquio syndrome a, Morquio Syndrome B, ossified ear cartilages (e.g., with mental deficiency, muscle wasting, and osteocraniostenosis), osteoporosis and oculocutaneous hypopigmentation syndrome, osteoporosis-pseudoglioma syndrome, juvenile osteoporosis, osteosclerosis with ichthyosis and fractures, ovarian dysgenesis 1, ovarian dysgenesis 2, ovarian dysgenesis 3, ovarian dysgenesis 4, pituitary adenoma, polycystic lipomembranous osteodysplasia with sclerosing leukoencephalopathy, Prader-Willi Habitus, osteopenia, Okamoto type premature aging syndrome, Prieto X-linked mental retardation syndrome, pycnodysostosis, Pyle Disease, Reifenstein Syndrome, autosomal dominant distal renal tubular acidosis, Type 1 Schwartz-Jampel Syndrome, Type 2 Schwartz-Jampel Syndrome, Type 3 Schwartz-Jampel Syndrome, Type 4 Schwartz-Jampel Syndrome, X-linked spondyloepiphyseal dysplasia tarda, and Torg-Winchester Syndrome.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Administration of a TGF-β Antagonist Conjugated to a Bone-Targeting Collagen-Binding Domain for the Treatment of Osteogenesis Imperfecta

Using the compositions and methods described herein, a physician of skill in the art can administer to a patient (e.g., a human patient) a conjugate containing a TGF-β antagonist peptide bound to a bone-targeting moiety to treat a disease associated with elevated TGF-β activity and/or elevated bone turnover relative to a healthy individual not suffering from the disease. For instance, a physician of skill in the art may assess a patient suffering from osteogenesis imperfecta by first evaluating the concentration of one or more biomarkers of bone turnover, such as serum and bone alkaline phosphatase, serum osteocalcin (sOC), serum type I collagen C-telopeptide breakdown products (sCTX), urinary free-deoxypyridinoline (ufDPD), and urinary cross-linked N-telopeptides of type I collagen (uNTX). A finding that one or more of these biomarkers is elevated may signal an elevated bone turnover rate, indicating that the patient may be particularly well suited for treatment with a TGF-β antagonist capable of localizing to bone tissue. A physician of skill in the art may additionally assess the patient's frequency of, and propensity for, bone fracture so as to monitor the progression of the disease during the course of treatment. The physician may administer to the patient a therapeutically effective amount (e.g., an amount sufficient to attenuate TGF-β signaling and/or to reduce bone turnover) of a conjugate containing a TGF-β antagonist bound to a bone-targeting moiety, for instance, by way of a human Fc domain (e.g., a human IgG, IgE, IgM, IgA, or IgD Fc domain). The TGF-β antagonist may be any antagonist described herein, such as, for instance, a CD109 peptide or fragment thereof as described herein. The bone-targeting moiety may be any bone-targeting moiety described herein, such as, for instance, a collagen-binding domain or a hydroxyapatite-binding domain as described herein.

The conjugate may be administered to the subject in one or more doses (e.g., 1, 2, 3, 4, 5, 6, 7, 8, 9, 10, 15, 20, 25, 30, or more) per a specified time interval, such as one or more doses per day, per week, per month, or per year. The patient may be evaluated between doses so as to monitor the effectiveness of the therapy and to increase or reduce the dosage based on the patient's response. For instance, a reduction in the incidence of bone fractures, an improved ability of the patient to walk, and/or a reduction in the concentration of one or more biomarkers of bone turnover in a sample isolated from the patient may indicate that the therapy is effectively treating the condition.

The therapy may be administered to the patient by a variety of routes of administration, for instance, as determined by a physician of skill in the art. For example, the therapy may be administered to the patient in one or more repeat doses subcutaneously, intradermally, intramuscularly, intraperitoneally, intravenously, or orally, or by nasal or by epidural administration.

Prior to the conclusion of therapy, the physician may prescribe progressively lower doses of the conjugate to the patient so as to gradually reduce the concentration of the therapy. The therapy may involve only a single dosing of the therapeutic conjugate. Alternatively, the therapy may continue, for instance, for a period of days, weeks, months, or years prior to completion.

### Example 2. Effects of Anti-TGF-β Antibody Conjugated to Decaaspartate Bone-Targeting Peptide on Viability, Mineralization, Collagen Deposition, and Alkaline Phosphatase Activity in Cultured Osteoblasts

### Osteoblast Cell Culture Assay

In vitro culturing of osteoblast cells is a convenient and useful method for the study of osteoblast activity that allows experimentation on biological processes related to bone formation in ways that are not always achievable in living organisms. Established cell lines provide a stable, homogeneous and reproducible model to investigate factors affecting osteoblast signaling, differentiation and mineralization. Cell culture models that produce an abundant, collagen- and noncollagenous protein-rich extracellular matrix—such as occurs in the MC3T3-E1 mouse pre-osteoblast cell line model—are particularly useful in that they allow fundamental questions about matrix mineralization to be queried in vitro. This work utilized the widely accepted cell culture model that is thought to closely resemble in many ways bone formation and mineralization in vivo, particularly with regard to extracellular matrix secretion, assembly and mineralization. This osteoblast cell culture system has been used for decades by many research groups, and has recently been further validated by a direct comparison to bone structure, composition and particularly mineralization (Ref. 1). Here, MC3T3-E1 osteoblasts were utilized to assess the inhibitory potential of TGF-β on mineralization of these bone cell cultures.

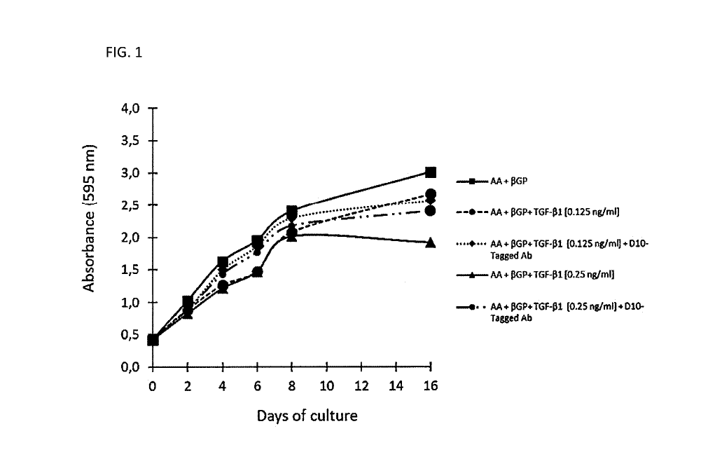

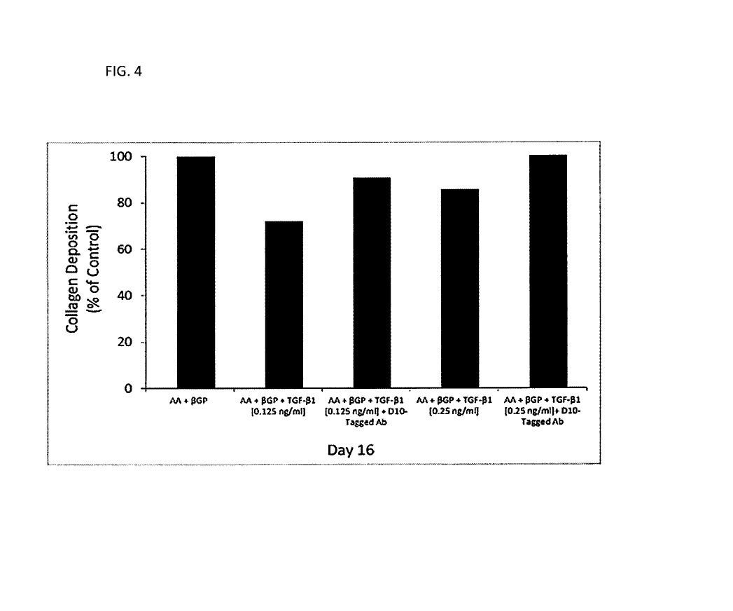

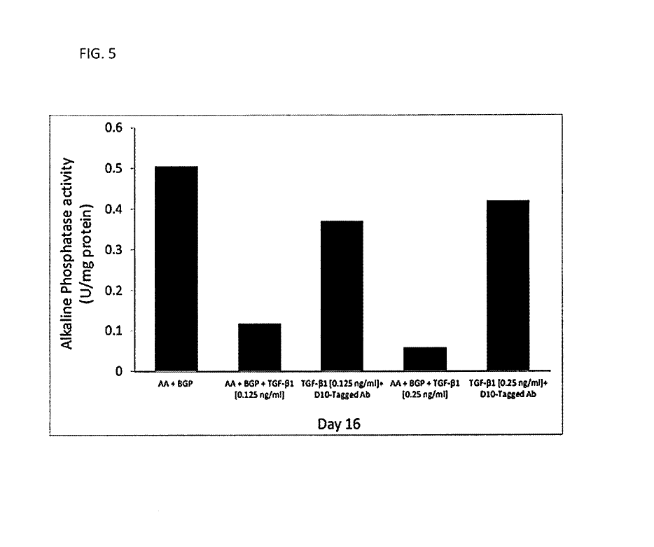

Cell cultures treated with TGF-β showed excellent maintenance of cell survival and activity (FIG. 1). The treated osteoblasts were viable and healthy, expressing variably markers of osteoblast differentiation, notably the biomarkers alkaline phosphatase and collagen commonly used to identify osteoblast activity, along with mineralization potential (FIGS. 1, 4, 5). Treatment of the cultures with TGF-β reduced the early osteoblast marker alkaline phosphatase activity compared to controls, a reduction that was rescued by the addition of neutralizing D10-Tagged antibody against TGF-β (FIG. 5). Collagen secretion and assembly is another important marker of osteoblast progression and differentiation, and cultures treated with TGF-β showed high levels of collagenous extracellular matrix assembly in the culture dishes. D10-Tagged antibody treatment had little effect on this prerequisite of collagen matrix for subsequent mineralization (FIG. 4). This means that an extracellular matrix is available for mineralization after all treatments.

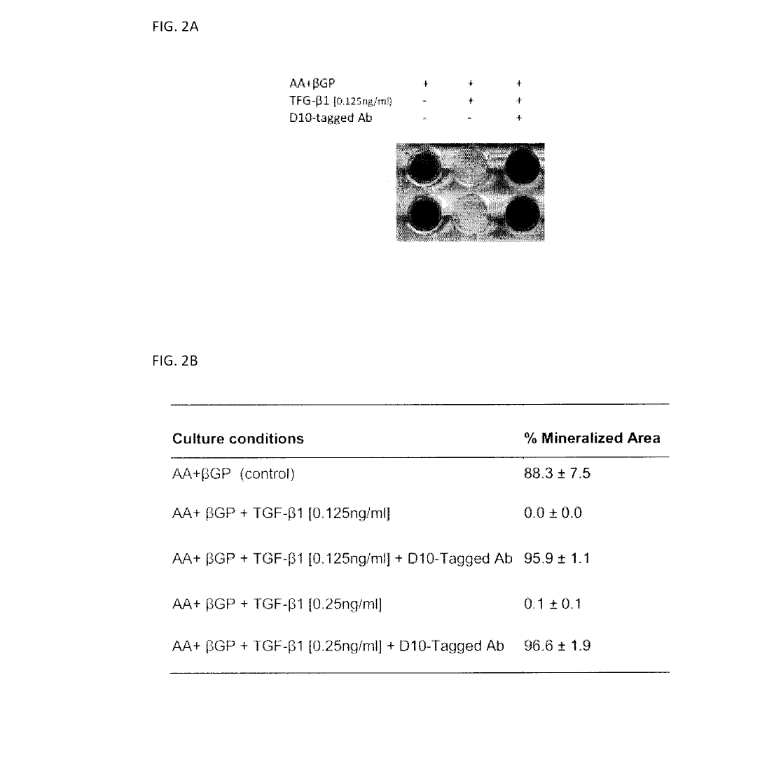

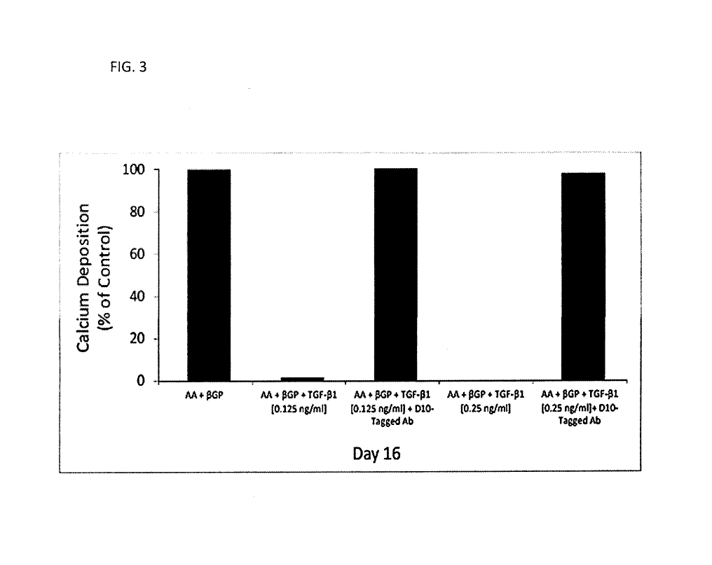

Mineralization of the osteoblast cultures was highly and significantly negatively affected by treatment with TGF-β. The treatment with TGF-β profoundly and essentially completely blocked mineralization of the cultures as assessed by von Kossa staining for mineral (FIGS. 2A and 2B) and calcium quantification (FIG. 3). This inhibitory effect on mineralization was completely rescued by the addition of neutralizing D10-Tagged antibody (FIGS. 2, 3).

### Mineral-Binding Assay

Using a cell-free, mineral-binding assay, direct assessment can be obtained for the binding of proteins to mineral. For this, the mineral of bone (a calcium-phosphate inorganic phase called hydroxyapatite) can be represented in vitro using highly pure synthetic mineral preparations that very closely approximate the mineral phase of bone. These preparations are readily amenable to protein binding assays using standard incubation steps and then assessment of mineral binding amounts using routine biochemical assays that typically measure depletion of the protein of interest from the solution (supernatant)(Refs. 2-4). These measurements all precise quantification of protein binding, and also direct visualization (imaging) approaches to be used.

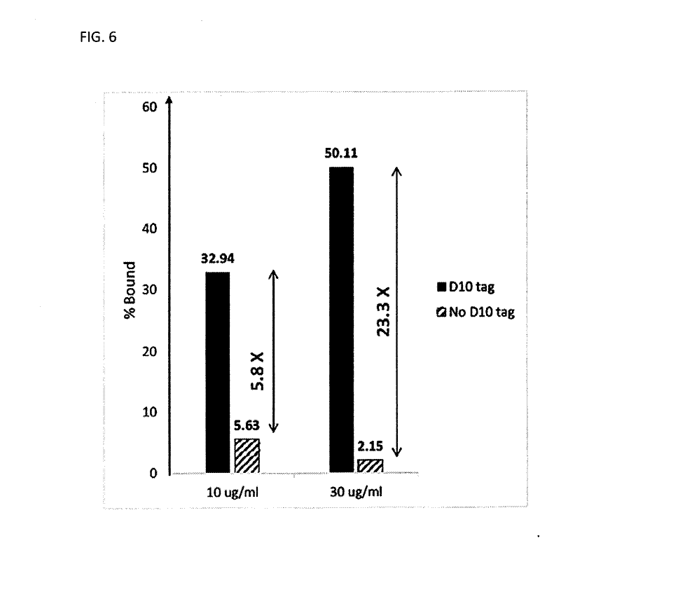

Comparative mineral-binding assays were performed using the D10-Tagged antibody and synthetic hydroxyapatite crystals, and bovine serum albumin measurements were also made to assess nonspecific binding of protein to mineral. Quantification of the mineral-binding reactions revealed that the D10-tagged antibody exhibited a markedly enhanced hydroxyapatite binding affinity relative to the untagged control antibody (1D11) (FIG. 6).

Taken together, the results of these experiments demonstrate the ability of a TGF-β antagonist conjugated to a bone-targeting moiety to ameliorate conditions associated with elevated bone turnover, such as osteogenesis imperfecta. TGF-β antagonists conjugated to bone-targeting moieties represent a useful paradigm for treating this and other skeletal disorders.

### Materials and Methods

### Cell Culture

Mouse calvarial pre-osteoblasts (MC3T3-E1, subclone 14) were cultured in minimum essential medium (MEM) lacking ascorbic acid (AA) and L-glutamine (Life Technology) supplemented with 10% fetal bovine serum (FBS) (Hyclone), 1% penicillin-streptomycin antibiotics, L-glutamine and L-aspartic in a humidified atmosphere at 37° C. and 5% CO₂.

For all experiments, cells were plated at 50,000 cells per cm². Cells differentiation into mature osteoblasts was induced 24 h after plating (day 0) by the addition of 50 μg/ml ascorbic acid (AA) plus 10 mM β-glycerophosphate (βGP) (Sigma) alone or with recombinant human Transforming Growth Factor Beta 1 (rhTGF-β1, R&D Systems) with/without 10 μg/ml custom-made D10-Tagged antibody (Genzyme). Cells were treated with medium plus supplements every second day for up to 16 days.

### Cell Viability

Cell viability was tested in the presence of all reagents by analyzing the incorporation of 3-(4,5-dimethyl-2-thiazolyl)-2,5-diphenyl-2H-tetrazolium bromide (MTT, Sigma) into viable cells at different time points (day 0, 2, 4, 6, 8 & 16).

### Osteoblast Differentiation and Mineralization Assays in Cell Culture

Mineralization assay by von Kossa (silver nitrate) staining and calcium kit quantification After 16 days of culture, cells were washed once with phosphate-buffered saline (PBS, Wisent), then fixed with 95% ethanol for 10 min at 37° C. Cell layer was hydrated twice with double-distilled H₂O, then incubated for an hour at 37° C. with 5% silver nitrate solution (von Kossa staining, Fisher). After incubation, silver nitrate solution was rinsed away and the cell culture wells were washed twice with distilled H₂O and exposed to direct light for 2 hours. Mineral deposits in the cultures turn brownish-black in color. Quantification of mineralization was done using scans of the wells and Image J software to record staining density.

Calcium concentrations were measured after an hour incubation from a 0.5 M HCl extract of the centrifuged cell/matrix pellet (day 6, 8 and 16) using a commercially available calcium kit (Sekisui Diagnostics).

### Collagen Type I Quantification by Picrosirius Red Staining

Cells were washed twice with PBS, then fixed with Bouin's fluid (75% saturated picric acid, 3.7% formaldehyde, 5% glacial acetic acid) for 1 h at room temperature. Cells were washed twice and incubated with distilled H₂O for 15 min to remove excess Bouin fluid. Collagen I (COL I) was stained by incubation with 2 mg/ml Sirius Red staining solution in saturated picric acid (Sigma) for 1 h at room temperature with gentle agitation, which stains collagen pink/red/purple, then the cells were exposed to two washes of 0.01 N HCl which removes unbound dye. For quantification of bound dye for collagen measurement, samples are dissolved in 0.1 N NaOH to release bound dye. For calibration, a standard curve of serial dilutions is generated using collagen type I from rat tail tendon (Sigma) that is plated and dried overnight and stained as above. Collagen standards and samples were run in triplicates and released stain amounts measured at 560 nm using spectrophotometer microplate reader. Collagen concentrations in the samples were calculated using the generated standard curve.

### Alkaline Phosphatase Enzyme Activity Assay

Alkaline phosphatase activity assay was measured from cells (day 6, 8 & 16 of culture) harvested and sonicated in 10 mM Tris-HCl (pH 7.4), 0.2% IGEBAL (Sigma) and 2 mM PMSF. Alkaline phosphatase activity was detected using a colorimetric method with p-nitrophenylphosphate (Sigma), an alkaline phosphatase substrate, with reference to a standard curve of alkaline phosphatase (Sigma) activity.

### Hydroxyapatite Mineral-Binding Assay

Equal amounts of hydroxyapatite crystals (Berkeley Advanced Biomaterials) in 20 mM Tris/HCl, 150 mM NaCl, at pH 7.4 with 0.1% Tween were incubated each with either 1D11 control antibody, D10-Tagged antibody (GC1008 conjugated to a decaaspartate peptide), or bovine serum albumin (BSA) for 1 h at room temperature, with constant shaking, followed by centrifugation at 10,000 g for 5 min. Protein concentration in the supernatant was measured in triplicate using the Micro BCA protein assay (Pierce). Absorbance was read spectrophotometrically in a microplate reader at 560 nm. The protein amount depleted from the supernatant was considered as the mineral-bound fraction.

### REFERENCES

1. Addison W N, Nelea V, Chicatun F, Chien Y C, Tran-Khanh N, Buschmann M D, Nazhat S N, Kaartinen M T, Vali H, Tecklenburg M M, Franceschi R T and McKee M D (2015) Extracellular matrix mineralization in murine MC3T3-E1 osteoblast cultures: An ultrastructural, compositional and comparative analysis with mouse bone. Bone 71:244-256.
2. Addison W N, Azari F, Sorensen E S, Kaartinen M T and McKee M D (2007) Pyrophosphate inhibits mineralization of osteoblast cultures by binding to mineral, upregulating osteopontin and inhibiting alkaline phosphatase activity. J. Biol. Chem., 282:15872-83.
3. Addison W N, Nakano Y, Loisel T, P. Crine and McKee M D (2008) MEPE-ASARM peptides control extracellular matrix mineralization by binding to hydroxyapatite: An inhibition regulated by PHEX cleavage of ASARM. J. Bone Miner. Res. 23:1638-1649.
4. McKee M D, Nakano Y, Masica D L, Gray J J, Lemire I, Heft R, Whyte M P, Crine P and Millan J L (2011) Enzyme replacement therapy prevents dental defects in a mouse model of hypophosphatasia. J. Dent. Res. 90:470-476.

### Example 3. Affinity of Anti-TGF-β Antibody Conjugated to Decaaspartate Bone-Targeting Peptide for Hydroxyapatite

The binding of bone-targeted anti-TGF-β antibody GC1008 and control non-targeted 1D11 antibody was examined by surface plasmon resonance using a BIACORE™ T200 instrument. Experiments were performed on CM5 sensor chips at 25° C. in PBS-Tween-20 (PBST) buffer. Lyophilized TGF-β was resuspended in 4 mM HCl (100 μg/mL) and then diluted to 1 μg/mL or 5 μg/mL in 10 mM sodium acetate pH 4.0 for immobilization using BIACORE™ Amine Coupling Kit. High density (˜3600 RU TGF-β1) and lower density (800 RU TGF-β2, 460 RU TGF-β3) TGF-β surfaces were achieved by adjusting TGF-β concentration and incubation times. The concentration of both bone-targeted and non-bone-targeted anti-TGF-β antibodies was 500 nM.

Corresponding reference surfaces were prepared in PBST without ligand. Injection parameters were 5 μL/min, contact time 180 s, dissociation time 600 s. The running buffer contained 10 mM glycine, and the sensor chip was regenerated in 10 mM glycine pH 1.5, 10 ul/min for 60 s. Surface plasmon resonance conditions used for these experiments are summarized in Tables 10 and 11, below.

TABLE 10
Surface plasmon resonance chip and sample conditions

| | |
|---|---|
| Chip | CM5 S series lot 1025131 (Expiry date 2018-05) |
| | Fresh immobilization of ligands on Jul. 28, 2018 |
| | to one chip: |
| | FC1 = control |
| | FC2 = 1001 RU TGFb2 |
| | FC3 = 851 RU TGFb1 |
| | FC4 = 872 RU TGFB3 |
| Concentration of | 500 nM |
| sample | |
| Condition sample | 5 ul/min, contact 180 s, dissociation 600 s |
| injection | |
| Condition regeneration | 10 mM glycine pH 1.5, 10 ul/min, 60 s |
| injection | |
| Running/Dilution | PBST running buffer |
| buffer | |
| Temperature | 25 C. |

TABLE 11
Surface plasmon resonance sample properties

| | Initial | Final | | Sample | PBST |
|---|---|---|---|---|---|
| Sample name | concentration | Concentration | | volume | volume |
| and lot | (nM) | (nM) | Final volume (uL) | (uL) | (uL) |
| 1D11 antibody | 52000 | 500 | 150 | 1.4 | 148.6 |
| (BioXcell | | | | | |
| Cat#BE0057) | | | | | |
| GC1008-D10 | 5533 | 500 | 150 | 13.6 | 136.4 |

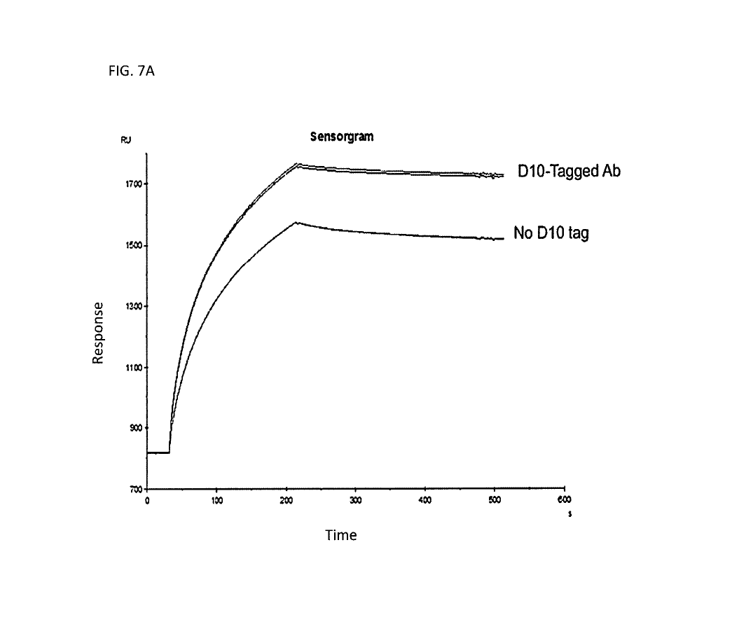

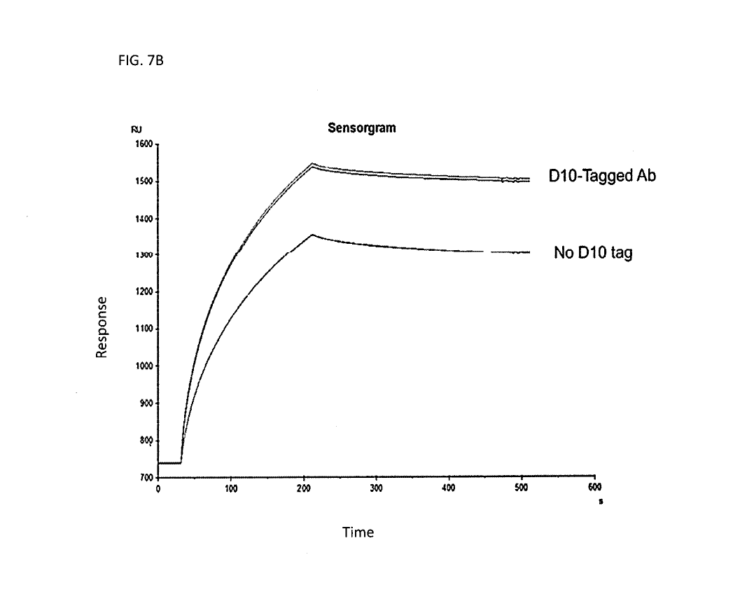

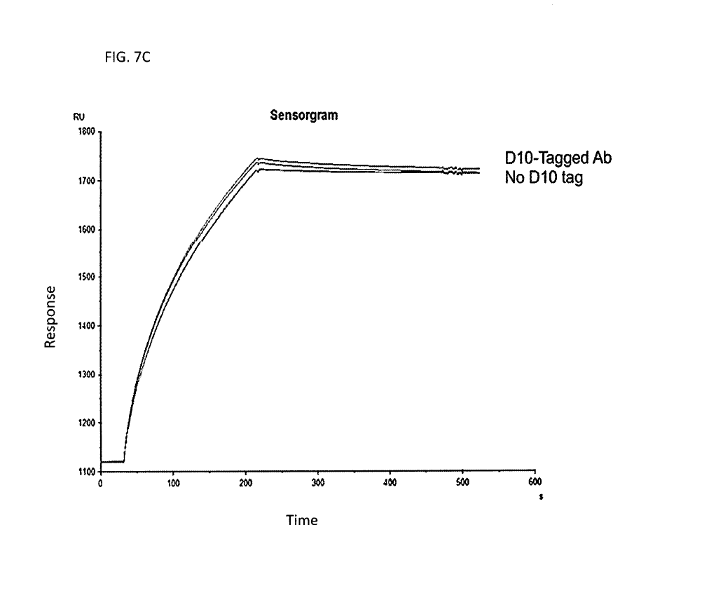

Using the methods described above, the affinity of anti-TGF-β antibody 1D11 alone, and the affinity of its humanized counterpart, GC1008, conjugated to decaaspartate (“GC1008-D10”), was determined for each of TGF-β isoforms TGF-β1, TGF-β2, and TGF-β3. As demonstrated in FIGS. 7A-7C, the GC1008-D10 conjugate exhibits high affinity for each of the TGF-β1, TGF-β2, and TGF-β3 isoforms. Particularly, GC1008-D10 binds TGF-β1 and TGF-β2 with a higher affinity than the unconjugated 1D11 antibody, and GC1008-D10 dissociates from TGF-β1 and TGF-β2 with a lower k_{off} relative to the unconjugated 1D11 antibody.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A conjugate comprising a TGF-β antagonist bound to a targeting moiety, wherein said targeting moiety binds a protein or mineral present in human bone tissue.

2. The conjugate of claim 1, wherein said TGF-β antagonist is an antibody, antigen-binding fragment thereof, protein, peptide, or small molecule that binds TGF-β.

3. The conjugate of claim 2, wherein said TGF-β antagonist is a peptide that binds TGF-β.

4. The conjugate of claim 3, wherein said peptide has the amino acid sequence IDGVYDNAEYAERFMEENEGHIVDIHDFSLGSS (SEQ ID NO: 5).

5. The conjugate of claim 3, wherein said peptide has the amino acid sequence WIWLDTNMGYRIYQEFEVT (SEQ ID NO: 1).

6. The conjugate of claim 3, wherein said peptide has the amino acid sequence of residues 21-1404 of SEQ ID NO: 2.

7. The conjugate of claim 3, wherein said peptide has the amino acid sequence of residues 21-1428 of SEQ ID NO: 2

8. The conjugate of claim 3, wherein said peptide has the amino acid sequence of SEQ ID NO: 2.

9. The conjugate of claim 3, wherein said peptide has the amino acid sequence WIWLDTNMGSRIYQEFEVT (SEQ ID NO: 3).

10. The conjugate of claim 3, wherein said peptide has the amino acid sequence of residues 21-1404 of SEQ ID NO: 4.

11. The conjugate of claim 3, wherein said peptide has the amino acid sequence of residues 21-1428 of SEQ ID NO: 4

12. The conjugate of claim 3, wherein said peptide has the amino acid sequence of SEQ ID NO: 4.

13. The conjugate of claim 3, wherein said peptide has the amino acid sequence of SEQ ID NO: 6.

14. The conjugate of claim 3, wherein said peptide has the amino acid sequence RKHFPETWIWLDTNMGYRIYQEFEV (SEQ ID NO: 7).

15. The conjugate of claim 3, wherein said peptide has an amino acid sequence selected from the group consisting of ANFCLGPCPYIWSLDT (SEQ ID NO: 8), ANFCSGPCPYLRSADT (SEQ ID NO: 9), PYIWSLDTQY (SEQ ID NO: 10), PYLWSSDTQH (SEQ ID NO: 11), PYLRSADTTH (SEQ ID NO: 12), WSXD (SEQ ID NO: 13), and RSXD (SEQ ID NO: 14), wherein X represents any naturally occurring amino acid.

16. The conjugate of claim 3, wherein said peptide has an amino acid sequence selected from the group consisting of TSLDATMIWTMM (SEQ ID NO: 15), SNPYSAFQVDIIVDI (SEQ ID NO: 16), TSLMIWTMM (SEQ ID NO: 17), TSLDASIIWAMMQN (SEQ ID NO: 18), SNPYSAFQVDITID (SEQ ID NO: 19), EAVLILQGPPYVSWL (SEQ ID NO: 20), and LDSLSFQLGLYLSPH (SEQ ID NO: 21).

17. The conjugate of claim 3, wherein said peptide has an amino acid sequence selected from the group consisting of TSLDASIIWAMMQN (SEQ ID NO: 22), KRIWFIPRSSWYERA (SEQ ID NO: 23), KRIWFIPRSSW (SEQ ID NO: 24), KRIWFIPRSSW (SEQ ID NO: 25), and KRIWFIPRSSW (SEQ ID NO: 26).

18. The conjugate of claim 3, wherein said peptide has the amino acid sequence of any one of SEQ ID NOs: 27-49.

19. The conjugate of claim 3, wherein said peptide has the amino acid sequence of SEQ ID NO: 50.

20. The conjugate of claim 2, wherein said TGF-β antagonist is an antibody or antigen-binding fragment thereof that binds TGF-β.

21. The conjugate of claim 20, wherein said antibody or antigen-binding fragment thereof comprises the following complementarity determining regions (CDRs):
a. a CDR-H1 having the amino acid sequence SNVIS (SEQ ID NO: 327);
b. a CDR-H2 having the amino acid sequence GVIPIVDIANYAQRFKG (SEQ ID NO: 328);
c. a CDR-H3 having the amino acid sequence TLGLVLDAMDY (SEQ ID NO: 329);
d. a CDR-L1 having the amino acid sequence RASQSLGSSYLA (SEQ ID NO: 330);
e. a CDR-L2 having the amino acid sequence GASSRAP (SEQ ID NO: 331); and
f. a CDR-L3 having the amino acid sequence QQYADSPIT (SEQ ID NO: 332).

22. The conjugate of claim 20, wherein said antibody or antigen-binding fragment thereof competitively inhibits the binding of TGF-β to a second antibody or antigen binding fragment thereof, wherein said second antibody or antigen-binding fragment thereof comprises the following CDRs:
a. a CDR-H1 having the amino acid sequence SNVIS (SEQ ID NO: 327);
b. a CDR-H2 having the amino acid sequence GVIPIVDIANYAQRFKG (SEQ ID NO: 328);
c. a CDR-H3 having the amino acid sequence TLGLVLDAMDY (SEQ ID NO: 329);
d. a CDR-L1 having the amino acid sequence RASQSLGSSYLA (SEQ ID NO: 330);
e. a CDR-L2 having the amino acid sequence GASSRAP (SEQ ID NO: 331); and
f. a CDR-L3 having the amino acid sequence QQYADSPIT (SEQ ID NO: 332).

23. The conjugate of any one of claims 20-22, wherein said antibody or antigen-binding fragment thereof comprises a heavy chain variable region having the amino acid sequence of SEQ ID NO: 333, or an amino acid sequence that is at least 85% identical thereto.

24. The conjugate of any one of claims 20-23, wherein said antibody or antigen-binding fragment thereof comprises a light chain variable region having the amino acid sequence of SEQ ID NO: 334, or an amino acid sequence that is at least 85% identical thereto.

25. The conjugate of any one of claims 20-24, wherein said antibody or antigen-binding fragment thereof is selected from the group consisting of a monoclonal antibody or antigen-binding fragment thereof, a polyclonal antibody or antigen-binding fragment thereof, a humanized antibody or antigen-binding fragment thereof, a bispecific antibody or antigen-binding fragment thereof, an optimized antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, and a tandem di-scFV.

26. The method of claim 25, wherein said antibody is a humanized antibody.

27. The method of claim 25, wherein said antibody is an optimized antibody.

28. The method of claim 27, wherein said optimized antibody is an affinity-matured antibody.

29. The method of any one of claims 1-28, wherein said antibody has an isotype selected from the group consisting of IgG, IgA, IgM, IgD, and IgE.

30. The conjugate of claim 1, wherein said TGF-β antagonist is a peptide that binds a TGF-β receptor.

31. The conjugate of claim 30, wherein said peptide has an amino acid sequence selected from the group consisting of HANFCLGPCPYIWSL (SEQ ID NO: 51), FCLGPCPYIWSLDT (SEQ ID NO: 52), and HEPKGYHANFCLGPCP (SEQ ID NO: 53).

32. The conjugate of any one of claims 1-31, wherein said targeting moiety is a peptide.

33. The conjugate of claim 32, wherein said targeting moiety is a peptide that binds a protein present in human bone tissue.

34. The conjugate of claim 33, wherein said protein present in human bone tissue is collagen.

35. The conjugate of claim 34, wherein said peptide that binds said protein has the amino acid sequence of any one of SEQ ID NOs: 54-56.

36. The conjugate of claim 34, wherein said peptide that binds said protein has the amino acid sequence of any one of SEQ ID NOs: 57-59.

37. The conjugate of claim 34, wherein said peptide that binds said protein has the amino acid sequence of SEQ ID NO: 56.

38. The conjugate of claim 33, wherein said targeting moiety is a peptide that binds a mineral present in human bone tissue.

39. The conjugate of claim 38, wherein said mineral present in human bone tissue is hydroxyapatite.

40. The conjugate of claim 39, wherein said targeting moiety is a polyanionic peptide.

41. The conjugate of claim 40, wherein said polyanionic peptide comprises one or more amino acids bearing a side-chain substituent selected from the group consisting of carboxylate, sulfonate, phosphonate, and phosphate.

42. The conjugate of claim 40 or 41, wherein said polyanionic peptide comprises one or more glutamate residues.

43. The conjugate of claim 42, wherein said polyanionic peptide comprises from 1 to 25 glutamate residues.

44. The conjugate of claim 43, wherein said polyanionic peptide comprises from 3 to 20 glutamate residues.

45. The conjugate of claim 44, wherein said polyanionic peptide comprises from 5 to 15 glutamate residues.

46. The conjugate of claim 45, wherein said polyanionic peptide comprises from 8 to 12 glutamate residues.

47. The conjugate of claim 46, wherein said polyanionic peptide comprises 10 glutamate residues.

48. The conjugate of claim 42, wherein said polyanionic peptide is a peptide of the formula Eₙ, wherein E designates a glutamate residue and n is an integer from 1 to 25.

49. The conjugate of claim 48, wherein n is an integer from 3 to 20.

50. The conjugate of claim 48, wherein n is an integer from 5 to 15.

51. The conjugate of claim 48, wherein n is an integer from 8 to 12.

52. The conjugate of claim 48, wherein n is 10.

53. The conjugate of any one of claims 42-47, wherein said glutamate residues are consecutive.

54. The conjugate of any one of claims 42-47, wherein said glutamate residues are discontinuous.

55. The conjugate of any one of claims 40-54, wherein said polyanionic peptide comprises one or more aspartate residues.

56. The conjugate of claim 55, wherein said polyanionic peptide comprises from 1 to 25 aspartate residues.

57. The conjugate of claim 56, wherein said polyanionic peptide comprises from 3 to 20 aspartate residues.

58. The conjugate of claim 57, wherein said polyanionic peptide comprises from 5 to 15 aspartate residues.

59. The conjugate of claim 58, wherein said polyanionic peptide comprises from 8 to 12 aspartate residues.

60. The conjugate of claim 59, wherein said polyanionic peptide comprises 10 aspartate residues.

61. The conjugate of claim 55, wherein said polyanionic peptide is a peptide of the formula Dₙ, wherein D designates an aspartate residue and n is an integer from 1 to 25.

62. The conjugate of claim 61, wherein n is an integer from 3 to 20.

63. The conjugate of claim 61, wherein n is an integer from 5 to 15.

64. The conjugate of claim 61, wherein n is an integer from 8 to 12.

65. The conjugate of claim 61, wherein n is 10.

66. The conjugate of any one of claims 55-60, wherein said aspartate residues are consecutive.

67. The conjugate of any one of claims 55-60, wherein said aspartate residues are discontinuous.

68. The conjugate of any one of claims 40-68, wherein the ratio of amino acids bearing a side-chain that is negatively-charged at physiological pH to the total quantity of amino acids in said polyanionic peptide is from about 0.5 to about 2.0.

69. The conjugate of claim 38, wherein said peptide that binds said mineral has the amino acid sequence of any one of SEQ ID NOs: 60-326.

70. The conjugate of any one of claims 1-69, wherein said TGF-β antagonist is bound to said targeting moiety by way of an immunoglobulin Fc domain.

71. The conjugate of claim 70, wherein said TGF-β antagonist is bound to the N-terminus of said immunoglobulin Fc domain and said targeting moiety is bound to the C-terminus of said immunoglobulin Fc domain.

72. The conjugate of claim 70, wherein said TGF-β antagonist is bound to the C-terminus of said immunoglobulin Fc domain and said targeting moiety is bound to the N-terminus of said immunoglobulin Fc domain.

73. The conjugate of any one of claims 70-72, wherein said immunoglobulin is selected from the group consisting of human IgG, human IgA, human IgM, human IgE, and human IgD.

74. A pharmaceutical composition comprising the conjugate of any one of claims 1-73 and a pharmaceutically acceptable excipient.

75. The pharmaceutical composition of claim 74, wherein said conjugate is formulated for subcutaneous, intradermal, intramuscular, intraperitoneal, intravenous, intranasal, epidural, or oral administration.

76. The pharmaceutical composition of claim 75, wherein said conjugate is formulated for intramuscular administration.

77. The pharmaceutical composition of claim 75, wherein said conjugate is formulated for intravenous administration.

78. The pharmaceutical composition of claim 75, wherein said conjugate is formulated for subcutaneous administration.

79. A method of treating a human patient suffering from a disease associated with elevated TGF-β signaling, said method comprising administering to said patient a therapeutically effective of the conjugate of any one of claims 1-73 or the pharmaceutical composition of any one of claims 74-78.

80. The method of claim 79, wherein said disease is a bone disease.

81. The method of claim 79 or 80, wherein said disease is selected from the group consisting of osteogenesis imperfecta, renal osteodystrophy, hyperparathyroid induced bone disease, diabetic bone disease, osteoarthritis, steroid induced bone disease, disuse osteoporosis, and Cerebral Palsy.

82. A method of treating a human patient suffering from a disease associated with elevated bone turnover, said method comprising administering to said patient a therapeutically effective of the conjugate of any one of claims 1-73 or the pharmaceutical composition of any one of claims 74-78.

83. The method of claim 79 or 82, wherein said disease is selected from the group consisting of osteogenesis imperfecta, McCune-Albright Syndrome, Gaucher Disease, Hyperoxaluria, Paget Disease of bone, and Juvenile Paget Disease.

84. The method of claim 81 or 83, wherein said disease is osteogenesis imperfecta.

85. The method of claim 84, wherein said osteogenesis imperfecta is Type I osteogenesis imperfecta, Type II osteogenesis imperfecta, Type III osteogenesis imperfecta, Type IV osteogenesis imperfecta, Type V osteogenesis imperfecta, Type VI osteogenesis imperfecta, Type VII osteogenesis imperfecta, Type VIII osteogenesis imperfecta, Type IX osteogenesis imperfecta, Type X osteogenesis imperfecta, or Type XI osteogenesis imperfecta.

86. The method of claim 79 or 82, wherein said disease is metastatic bone cancer.

87. The method of claim 86, wherein said patient is suffering from breast cancer or prostate cancer.

88. The method of claim 79 or 82, wherein said disease is selected from the group consisting of osteoporosis, fibrous dysplasia, Calmurati-Engleman Disease, Marfan's Syndrome, osteoglophonic dysplasia, autosomal dominant osteopetrosis, osteoporosis, osteoporosis-pseudoglioma syndrome, juvenile, geroderma osteodysplastica, osteogenesis imperfecta congenita, microcephaly, and cataracts.

89. The method of claim 79 or 82, wherein said disease is selected from the group consisting of pseudohypoparathyroidism, Cleidocranial Dysplasia, Dyskeratosis Congenita, Exudative Vitreoretinopathy 1, Schimmelpenning-Feuerstein-Mims Syndrome, Prader-Willi Syndrome, Achondrogenesis, Antley-Bixler Syndrome, Aspartylglucosaminuria, Celiac Disease, Cerebrooculofacioskeletal Syndrome 1, Lysinuric Protein Intolerance, neuropathy, dyskeratosis congenita, Ehlers-Danlos Syndrome, epiphyseal dysplasia, hyaline fibromatosis syndrome, Perrault Syndrome 1, hemochromatosis, homocystinuria (e.g., due to cystathionine beta-synthase deficiency), hypophosphatemic rickets with hypercalciuria, desbuquois dysplasia, multiple pterygium syndrome, lethal congenital contracture syndrome 1, mitochondrial DNA depletion Ssndrome 6 (hepatocerebral Type), Niemann-Pick Disease, osteopetrosis, porphyria, Rothmund-Thomson Syndrome, Wilson Disease, Dent Disease 1, occipital horn syndrome, hyperglycerolemia, hypophosphatemic rickets, Lowe Oculocerebrorenal Syndrome, renal tubulopathy, diabetes mellitus, cerebellar ataxia, vitamin D hydroxylation-deficient rickets, Warburg micro syndrome 1, Stuve-Wiedemann Syndrome, Blue Rubber Bleb Nevus syndrome, Singleton-Merten Syndrome, microcephalic osteodysplastic primordial dwarfism, dysosteosclerosis, Hallermann-Streiff Syndrome, Bruck Syndrome 1, multiple pterygium syndrome (e.g., X-Linked), spondylometaphyseal dysplasia with dentinogenesis imperfecta, Hall-Riggs Mental Retardation Syndrome, infantile multisystem neurologic disease with osseous fragility, acrocephalopolysyndactyly Type III, acroosteolysis, ACTH-independent macronodular adrenal hyperplasia, amino aciduria with mental deficiency, arthropathy, bone fragility (e.g., with craniosynostosis, ocular proptosis, hydrocephalus, and distinctive facial features), brittle cornea syndrome, cerebrotendinous xanthomatosis, Cri-Du-Chat Syndrome, dysplasia epiphysealis hemimelica, autosomal dominant Ehlers-Danlos Syndrome, familial osteodysplasia, Flynn-Aird Syndrome, geroderma osteodysplastica, glycogen storage disease Ia, Hutchinson-Gilford Progeria Syndrome, Infantile Systemic Hyalinosis, hypertrichotic osteochondrodysplasia, hyperzincemia with functional zinc depletion, hypophosphatasia, autosomal dominant hypophosphatemic rickets, X-linked recessive hypophosphatemic rickets, Lichtenstein Syndrome, macroepiphyseal dysplasia (e.g., with osteoporosis wrinkled skin, and agedappearance), Menkes Disease, Mental Retardation (e.g., X-Linked, Snyder-Robinson type), Jansen type metaphyseal chondrodysplasia, microspherophakia-metaphyseal dysplasia, morquio syndrome a, Morquio Syndrome B, ossified ear cartilages (e.g., with mental deficiency, muscle wasting, and osteocraniostenosis), osteoporosis and oculocutaneous hypopigmentation syndrome, osteoporosis-pseudoglioma syndrome, juvenile osteoporosis, osteosclerosis with ichthyosis and fractures, ovarian dysgenesis 1, ovarian dysgenesis 2, ovarian dysgenesis 3, ovarian dysgenesis 4, pituitary adenoma, polycystic lipomembranous osteodysplasia with sclerosing leukoencephalopathy, Prader-Willi Habitus, osteopenia, Okamoto type premature aging syndrome, Prieto X-linked mental retardation syndrome, pycnodysostosis, Pyle Disease, Reifenstein Syndrome, autosomal dominant distal renal tubular acidosis, Type 1 Schwartz-Jampel Syndrome, Type 2 Schwartz-Jampel Syndrome, Type 3 Schwartz-Jampel Syndrome, Type 4 Schwartz-Jampel Syndrome, X-linked spondyloepiphyseal dysplasia tarda, and Torg-Winchester Syndrome.

90. The method of any one of claims 79-89, said method comprising administering said conjugate or pharmaceutical composition to said patient subcutaneously, intradermally, intramuscularly, intraperitoneally, intravenously, or orally, or by nasal or by epidural administration.

91. The method of claim 90, said method comprising administering said conjugate or pharmaceutical composition to said patient intramuscularly.

92. The method of claim 90, said method comprising administering said conjugate or pharmaceutical composition to said patient intravenously.

93. The method of claim 90, said method comprising administering said conjugate or pharmaceutical composition to said patient subcutaneously.

94. A kit comprising the conjugate of any one of claims 1-73 and a package insert, wherein said package insert instructs a user of said kit to treat a human patient suffering from a disease associated with elevated TGF-β signaling by administering to said patient a therapeutically effective amount of said conjugate.

95. A kit comprising the pharmaceutical composition of any one of claims 74-78 and a package insert, wherein said package insert instructs a user of said kit to treat a human patient suffering from a disease associated with elevated bone turnover by administering to said patient a therapeutically effective amount of said conjugate.


SEQUENCE LISTING (337 sequences)

SEQ ID NO: 1 - protein, 19 aa, Artificial Sequence
  synthetic construct
WIWLDTNMGYRIYQEFEVT

SEQ ID NO: 2 - protein, 1428 aa, Artificial Sequence
  synthetic construct
MQGPPLLTAAHLLCVCTAALAVAPGPRFLVTAPGIIRPGGNVTIGVELLEHCPSQVTVKAELLKTASNLTVSVLEAEGVFEKGSFKTLTLPSLPLNSADEIYELRVTGRTQDEILFSNSTRLSFETKRISVFIQTDKALYKPKQEVKFRIVTLFSDFKPYKTSLNILIKDPKSNLIQQWLSQQSDLGVISKTFQLSSHPILGDWSIQVQVNDQTYYQSFQVSEYVLPKFEVTLQTPLYCSMNSKHLNGTITAKYTYGKPVKGDVTLTFLPLSFWGKKKNITKTFKINGSANFSFNDEEMKNVMDSSNGLSEYLDLSSPGPVEILTTVTESVTGISRNVSTNVFFKQHDYIIEFFDYTTVLKPSLNFTATVKVTRADGNQLTLEERRNNVVITVTQRNYTEYWSGSNSGNQKMEAVQKINYTVPQSGTFKIEFPILEDSSELQLKAYFLGSKSSMAVHSLFKSPSKTYIQLKTRDENIKVGSPFELVVSGNKRLKELSYMVVSRGQLVAVGKQNSTMFSLTPENSWTPKACVIVYYIEDDGEIISDVLKIPVQLVFKNKIKLYWSKVKAEPSEKVSLRISVTQPDSIVGIVAVDKSVNLMNASNDITMENVVHELELYNTGYYLGMFMNSFAVFQECGLWVLTDANLTKDYIDGVYDNAEYAERFMEENEGHIVDIHDFSLGSSPHVRKHFPETWIWLDTNMGYRIYQEFEVTVPDSITSWVATGFVISEDLGLGLTTTPVELQAFQPFFIFLNLPYSVIRGEEFALEITIFNYLKDATEVKVIIEKSDKFDILMTSNEINATGHQQTLLVPSEDGATVLFPIRPTHLGEIPITVTALSPTASDAVTQMILVKAEGIEKSYSQSILLDLTDNRLQSTLKTLSFSFPPNTVTGSERVQITAIGDVLGPSINGLASLIRMPYGCGEQNMINFAPNIYILDYLTKKKQLTDNLKEKALSFMRQGYQRELLYQREDGSFSAFGNYDPSGSTWLSAFVLRCFLEADPYIDIDQNVLHRTYTWLKGHQKSNGEFWDPGRVIHSELQGGNKSPVTLTAYIVTSLLGYRKYQPNIDVQESIHFLESEFSRGISDNYTLALITYALSSVGSPKAKEALNMLTWRAEQEGGMQFWVSSESKLSDSWQPRSLDIEVAAYALLSHFLQFQTSEGIPIMRWLSRQRNSLGGFASTQDTTVALKALSEFAALMNTERTNIQVTVTGPSSPSPLAVVQPTAVNISANGFGFAICQLNVVYNVKASGSSRRRRSIQNQEAFDLDVAVKENKDDLNHVDLNVCTSFSGPGRSGMALMEVNLLSGFMVPSEAISLSETVKKVEYDHGKLNLYLDSVNETQFCVNIPAVRNFKVSNTQDASVSIVDYYEPRRQAVRSYNSEVKLSSCDLCSDVQGCRPCEDGASGSHHHSSVIFIFCFKLLYFMELWL

SEQ ID NO: 3 - protein, 19 aa, Artificial Sequence
  synthetic construct
WIWLDTNMGSRIYQEFEVT

SEQ ID NO: 4 - protein, 1428 aa, Artificial Sequence
  synthetic construct
MQGPPLLTAAHLLCVCTAALAVAPGPRFLVTAPGIIRPGGNVTIGVELLEHCPSQVTVKAELLKTASNLTVSVLEAEGVFEKGSFKTLTLPSLPLNSADEIYELRVTGRTQDEILFSNSTRLSFETKRISVFIQTDKALYKPKQEVKFRIVTLFSDFKPYKTSLNILIKDPKSNLIQQWLSQQSDLGVISKTFQLSSHPILGDWSIQVQVNDQTYYQSFQVSEYVLPKFEVTLQTPLYCSMNSKHLNGTITAKYTYGKPVKGDVTLTFLPLSFWGKKKNITKTFKINGSANFSFNDEEMKNVMDSSNGLSEYLDLSSPGPVEILTTVTESVTGISRNVSTNVFFKQHDYIIEFFDYTTVLKPSLNFTATVKVTRADGNQLTLEERRNNVVITVTQRNYTEYWSGSNSGNQKMEAVQKINYTVPQSGTFKIEFPILEDSSELQLKAYFLGSKSSMAVHSLFKSPSKTYIQLKTRDENIKVGSPFELVVSGNKRLKELSYMVVSRGQLVAVGKQNSTMFSLTPENSWTPKACVIVYYIEDDGEIISDVLKIPVQLVFKNKIKLYWSKVKAEPSEKVSLRISVTQPDSIVGIVAVDKSVNLMNASNDITMENVVHELELYNTGYYLGMFMNSFAVFQECGLWVLTDANLTKDYIDGVYDNAEYAERFMEENEGHIVDIHDFSLGSSPHVRKHFPETWIWLDTNMGSRIYQEFEVTVPDSITSWVATGFVISEDLGLGLTTTPVELQAFQPFFIFLNLPYSVIRGEEFALEITIFNYLKDATEVKVIIEKSDKFDILMTSNEINATGHQQTLLVPSEDGATVLFPIRPTHLGEIPITVTALSPTASDAVTQMILVKAEGIEKSYSQSILLDLTDNRLQSTLKTLSFSFPPNTVTGSERVQITAIGDVLGPSINGLASLIRMPYGCGEQNMINFAPNIYILDYLTKKKQLTDNLKEKALSFMRQGYQRELLYQREDGSFSAFGNYDPSGSTWLSAFVLRCFLEADPYIDIDQNVLHRTYTWLKGHQKSNGEFWDPGRVIHSELQGGNKSPVTLTAYIVTSLLGYRKYQPNIDVQESIHFLESEFSRGISDNYTLALITYALSSVGSPKAKEALNMLTWRAEQEGGMQFWVSSESKLSDSWQPRSLDIEVAAYALLSHFLQFQTSEGIPIMRWLSRQRNSLGGFASTQDTTVALKALSEFAALMNTERTNIQVTVTGPSSPSPLAVVQPTAVNISANGFGFAICQLNVVYNVKASGSSRRRRSIQNQEAFDLDVAVKENKDDLNHVDLNVCTSFSGPGRSGMALMEVNLLSGFMVPSEAISLSETVKKVEYDHGKLNLYLDSVNETQFCVNIPAVRNFKVSNTQDASVSIVDYYEPRRQAVRSYNSEVKLSSCDLCSDVQGCRPCEDGASGSHHHSSVIFIFCFKLLYFMELWL

SEQ ID NO: 5 - protein, 33 aa, Artificial Sequence
  synthetic construct
IDGVYDNAEYAERFMEENEGHIVDIHDFSLGSS

SEQ ID NO: 6 - protein, 161 aa, Artificial Sequence
  synthetic construct
TMENVVHELELYNTGYYLGMFMNSFAVFQECGLWVLTDANLTKDYIDGVYDNAEYAERFMEENEGHIVDIHDFSLGSSPHVRKHFPETWIWLDTNMGSRIYQEFEVTVPDSITSWVATGFVISEDLGLGLTTTPVELQAFQPFFIFLNLPYSVIRGEEFAL

SEQ ID NO: 7 - protein, 25 aa, Artificial Sequence
  synthetic construct
RKHFPETWIWLDTNMGYRIYQEFEV

SEQ ID NO: 8 - protein, 16 aa, Artificial Sequence
  synthetic construct
ANFCLGPCPYIWSLDT

SEQ ID NO: 9 - protein, 16 aa, Artificial Sequence
  synthetic construct
ANFCSGPCPYLRSADT

SEQ ID NO: 10 - protein, 10 aa, Artificial Sequence
  synthetic construct
PYIWSLDTQY

SEQ ID NO: 11 - protein, 10 aa, Artificial Sequence
  synthetic construct
PYLWSSDTQH

SEQ ID NO: 12 - protein, 10 aa, Artificial Sequence
  synthetic construct
PYLRSADTTH

SEQ ID NO: 13 - protein, 4 aa, Artificial Sequence
  synthetic construct
  MISC_FEATURE (3)..(3): Xaa is any amino acid
WSXD

SEQ ID NO: 14 - protein, 4 aa, Artificial Sequence
  synthetic construct
  MISC_FEATURE (3)..(3): Xaa is any amino acid
RSXD

SEQ ID NO: 15 - protein, 12 aa, Artificial Sequence
  synthetic construct
TSLDATMIWTMM

SEQ ID NO: 16 - protein, 15 aa, Artificial Sequence
  synthetic construct
SNPYSAFQVDIIVDI

SEQ ID NO: 17 - protein, 9 aa, Artificial Sequence
  synthetic construct
TSLMIWTMM

SEQ ID NO: 18 - protein, 14 aa, Artificial Sequence
  synthetic construct
TSLDASIIWAMMQN

SEQ ID NO: 19 - protein, 14 aa, Artificial Sequence
  synthetic construct
SNPYSAFQVDITID

SEQ ID NO: 20 - protein, 15 aa, Artificial Sequence
  synthetic construct
EAVLILQGPPYVSWL

SEQ ID NO: 21 - protein, 15 aa, Artificial Sequence
  synthetic construct
LDSLSFQLGLYLSPH

SEQ ID NO: 22 - protein, 14 aa, Artificial Sequence
  synthetic construct
TSLDASIIWAMMQN

SEQ ID NO: 23 - protein, 15 aa, Artificial Sequence
  synthetic construct
KRIWFIPRSSWYERA

SEQ ID NO: 24 - protein, 11 aa, Artificial Sequence
  synthetic construct
KRIWFIPRSSW

SEQ ID NO: 25 - protein, 11 aa, Artificial Sequence
  synthetic construct
KRIWFIPRSSW

SEQ ID NO: 26 - protein, 11 aa, Artificial Sequence
  synthetic construct
KRIWFIPRSSW

SEQ ID NO: 27 - protein, 15 aa, Artificial Sequence
  synthetic construct
HANFCLGPCPYIWSL

SEQ ID NO: 28 - protein, 14 aa, Artificial Sequence
  synthetic construct
FCLGPCPYIWSLDT

SEQ ID NO: 29 - protein, 12 aa, Artificial Sequence
  synthetic construct
TSLDATMIWTMM

SEQ ID NO: 30 - protein, 15 aa, Artificial Sequence
  synthetic construct
SNPYSAFQVDIIVDI

SEQ ID NO: 31 - protein, 9 aa, Artificial Sequence
  synthetic construct
TSLMIWTMM

SEQ ID NO: 32 - protein, 14 aa, Artificial Sequence
  synthetic construct
TSLDASIIWAMMQN

SEQ ID NO: 33 - protein, 14 aa, Artificial Sequence
  synthetic construct
SNPYSAFQVDITID

SEQ ID NO: 34 - protein, 15 aa, Artificial Sequence
  synthetic construct
EAVLILQGPPYVSWL

SEQ ID NO: 35 - protein, 15 aa, Artificial Sequence
  synthetic construct
LDSLSFQLGLYLSPH

SEQ ID NO: 36 - protein, 23 aa, Artificial Sequence
  synthetic construct
HEPKGYHANFCLGPCPYIWSLDT

SEQ ID NO: 37 - protein, 15 aa, Artificial Sequence
  synthetic construct
WHKYFLRRPLSVRTR

SEQ ID NO: 38 - protein, 15 aa, Artificial Sequence
  synthetic construct
RFFTRFPWHYHASRL

SEQ ID NO: 39 - protein, 15 aa, Artificial Sequence
  synthetic construct
RKWFLQHRRMPVSVL

SEQ ID NO: 40 - protein, 15 aa, Artificial Sequence
  synthetic construct
SGRRHLHRHHIFSLP

SEQ ID NO: 41 - protein, 15 aa, Artificial Sequence
  synthetic construct
RLAHSHRHRSHVALT

SEQ ID NO: 42 - protein, 15 aa, Artificial Sequence
  synthetic construct
PPYHRFWRGHRHAVQ

SEQ ID NO: 43 - protein, 15 aa, Artificial Sequence
  synthetic construct
KRIWFIPRSSWYERA

SEQ ID NO: 44 - protein, 15 aa, Artificial Sequence
  synthetic construct
MPLSRYWWLFSHRPR

SEQ ID NO: 45 - protein, 14 aa, Artificial Sequence
  synthetic construct
KRIWFIPRSSWYER

SEQ ID NO: 46 - protein, 12 aa, Artificial Sequence
  synthetic construct
KRIWFIPRSSWY

SEQ ID NO: 47 - protein, 11 aa, Artificial Sequence
  synthetic construct
KRIWFIPRSSW

SEQ ID NO: 48 - protein, 11 aa, Artificial Sequence
  synthetic construct
KRIWFIPRSSW

SEQ ID NO: 49 - protein, 11 aa, Artificial Sequence
  synthetic construct
KRIWFIPRSSW

SEQ ID NO: 50 - protein, 662 aa, Artificial Sequence
  synthetic construct
MRPQILLLLALLTLGLAAQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNINLIIILTFILVSAILLTTLAACCCVRRQKFNQQYKA

SEQ ID NO: 51 - protein, 15 aa, Artificial Sequence
  synthetic construct
HANFCLGPCPYIWSL

SEQ ID NO: 52 - protein, 14 aa, Artificial Sequence
  synthetic construct
FCLGPCPYIWSLDT

SEQ ID NO: 53 - protein, 16 aa, Artificial Sequence
  synthetic construct
HEPKGYHANFCLGPCP

SEQ ID NO: 54 - protein, 121 aa, Artificial Sequence
  synthetic construct
PVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 55 - protein, 215 aa, Artificial Sequence
  synthetic construct
EIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 56 - protein, 183 aa, Artificial Sequence
  synthetic construct
QSGPLPKPSLQALPSSLVPLEKPVTLRCQGPPGVDLYRLEKLSSSRYQDQAVLFIPAMKRSLAGRYRCSYQNGSLWSLPSDQLELVATGVFAKPSLSAQPGPAVSSGGDVTLQCQTRYGFDQFALYKEGDPAPYKNPERWYRASFPIITVTAAHSGTYRCYSFSSRDPYLWSAPSDPLELVVT

SEQ ID NO: 57 - protein, 28 aa, Artificial Sequence
  synthetic construct
NGVFKYRPRYFLYKHAYFYPPLKRFPVQ

SEQ ID NO: 58 - protein, 14 aa, Artificial Sequence
  synthetic construct
NGVFKYRPRYFLYK

SEQ ID NO: 59 - protein, 14 aa, Artificial Sequence
  synthetic construct
HAYFYPPLKRFPVQ

SEQ ID NO: 60 - protein, 7 aa, Artificial Sequence
  synthetic construct
RPHTITN

SEQ ID NO: 61 - protein, 7 aa, Artificial Sequence
  synthetic construct
QSSYNPI

SEQ ID NO: 62 - protein, 7 aa, Artificial Sequence
  synthetic construct
QTHARHQ

SEQ ID NO: 63 - protein, 7 aa, Artificial Sequence
  synthetic construct
ETRTQLL

SEQ ID NO: 64 - protein, 7 aa, Artificial Sequence
  synthetic construct
HHQRSPA

SEQ ID NO: 65 - protein, 7 aa, Artificial Sequence
  synthetic construct
LQKSPSL

SEQ ID NO: 66 - protein, 7 aa, Artificial Sequence
  synthetic construct
PPKDSRG

SEQ ID NO: 67 - protein, 7 aa, Artificial Sequence
  synthetic construct
SAKKVFS

SEQ ID NO: 68 - protein, 7 aa, Artificial Sequence
  synthetic construct
SQHSTQD

SEQ ID NO: 69 - protein, 7 aa, Artificial Sequence
  synthetic construct
TIHSKPA

SEQ ID NO: 70 - protein, 7 aa, Artificial Sequence
  synthetic construct
TKDWLPS

SEQ ID NO: 71 - protein, 7 aa, Artificial Sequence
  synthetic construct
ANPPLSL

SEQ ID NO: 72 - protein, 7 aa, Artificial Sequence
  synthetic construct
AKQTVPV

SEQ ID NO: 73 - protein, 7 aa, Artificial Sequence
  synthetic construct
ATFSPPL

SEQ ID NO: 74 - protein, 7 aa, Artificial Sequence
  synthetic construct
DQYWGLR

SEQ ID NO: 75 - protein, 7 aa, Artificial Sequence
  synthetic construct
EPNHTRF

SEQ ID NO: 76 - protein, 7 aa, Artificial Sequence
  synthetic construct
HMLAQTF

SEQ ID NO: 77 - protein, 7 aa, Artificial Sequence
  synthetic construct
IGYPVLP

SEQ ID NO: 78 - protein, 7 aa, Artificial Sequence
  synthetic construct
KLSAWSF

SEQ ID NO: 79 - protein, 7 aa, Artificial Sequence
  synthetic construct
MYPLPAP

SEQ ID NO: 80 - protein, 7 aa, Artificial Sequence
  synthetic construct
FTLPTIR

SEQ ID NO: 81 - protein, 7 aa, Artificial Sequence
  synthetic construct
SMAAKSS

SEQ ID NO: 82 - protein, 7 aa, Artificial Sequence
  synthetic construct
SMYDTHS

SEQ ID NO: 83 - protein, 7 aa, Artificial Sequence
  synthetic construct
STLASMR

SEQ ID NO: 84 - protein, 7 aa, Artificial Sequence
  synthetic construct
TLMTTPP

SEQ ID NO: 85 - protein, 7 aa, Artificial Sequence
  synthetic construct
WLPPRTQ

SEQ ID NO: 86 - protein, 12 aa, Artificial Sequence
  synthetic construct
RTPLQPLEDFRP

SEQ ID NO: 87 - protein, 12 aa, Artificial Sequence
  synthetic construct
NTTTDIPSPSQF

SEQ ID NO: 88 - protein, 12 aa, Artificial Sequence
  synthetic construct
TLDKYTRLLSRY

SEQ ID NO: 89 - protein, 12 aa, Artificial Sequence
  synthetic construct
YPIMSHTCCHGV

SEQ ID NO: 90 - protein, 7 aa, Artificial Sequence
  synthetic construct
YEPAAAE

SEQ ID NO: 91 - protein, 7 aa, Artificial Sequence
  synthetic construct
ANPYHRH

SEQ ID NO: 92 - protein, 7 aa, Artificial Sequence
  synthetic construct
ASGPTNV

SEQ ID NO: 93 - protein, 7 aa, Artificial Sequence
  synthetic construct
QNYLLPK

SEQ ID NO: 94 - protein, 7 aa, Artificial Sequence
  synthetic construct
GTQTPQP

SEQ ID NO: 95 - protein, 7 aa, Artificial Sequence
  synthetic construct
HSTGPTR

SEQ ID NO: 96 - protein, 7 aa, Artificial Sequence
  synthetic construct
LSKNPLL

SEQ ID NO: 97 - protein, 7 aa, Artificial Sequence
  synthetic construct
LSKNPLL

SEQ ID NO: 98 - protein, 7 aa, Artificial Sequence
  synthetic construct
KLHASLA

SEQ ID NO: 99 - protein, 7 aa, Artificial Sequence
  synthetic construct
PLTQPSH

SEQ ID NO: 100 - protein, 7 aa, Artificial Sequence
  synthetic construct
PHNPGKL

SEQ ID NO: 101 - protein, 7 aa, Artificial Sequence
  synthetic construct
PTTMTRW

SEQ ID NO: 102 - protein, 7 aa, Artificial Sequence
  synthetic construct
VHLTHGQ

SEQ ID NO: 103 - protein, 7 aa, Artificial Sequence
  synthetic construct
TLAPTFR

SEQ ID NO: 104 - protein, 7 aa, Artificial Sequence
  synthetic construct
VHPRPSL

SEQ ID NO: 105 - protein, 7 aa, Artificial Sequence
  synthetic construct
TLLRTQV

SEQ ID NO: 106 - protein, 7 aa, Artificial Sequence
  synthetic construct
SSPPRVY

SEQ ID NO: 107 - protein, 7 aa, Artificial Sequence
  synthetic construct
SSVPGRP

SEQ ID NO: 108 - protein, 7 aa, Artificial Sequence
  synthetic construct
LPFQPPI

SEQ ID NO: 109 - protein, 7 aa, Artificial Sequence
  synthetic construct
IQHQAKT

SEQ ID NO: 110 - protein, 12 aa, Artificial Sequence
  synthetic construct
LPRDLHATPQQI

SEQ ID NO: 111 - protein, 12 aa, Artificial Sequence
  synthetic construct
LTPTMFNMHGVL

SEQ ID NO: 112 - protein, 12 aa, Artificial Sequence
  synthetic construct
SIPKMIPTESLL

SEQ ID NO: 113 - protein, 12 aa, Artificial Sequence
  synthetic construct
SFQSMSLMTLVV

SEQ ID NO: 114 - protein, 12 aa, Artificial Sequence
  synthetic construct
TQTWPQSSSHGL

SEQ ID NO: 115 - protein, 6 aa, Artificial Sequence
  synthetic construct
YELQMP

SEQ ID NO: 116 - protein, 12 aa, Artificial Sequence
  synthetic construct
AMSQTMTAAIEK

SEQ ID NO: 117 - protein, 12 aa, Artificial Sequence
  synthetic construct
GSAGLKYPLYKS

SEQ ID NO: 118 - protein, 12 aa, Artificial Sequence
  synthetic construct
INFQFLKPSTTR

SEQ ID NO: 119 - protein, 7 aa, Artificial Sequence
  synthetic construct
RHTLPLH

SEQ ID NO: 120 - protein, 7 aa, Artificial Sequence
  synthetic construct
NFAMNLR

SEQ ID NO: 121 - protein, 7 aa, Artificial Sequence
  synthetic construct
NFAMNLR

SEQ ID NO: 122 - protein, 7 aa, Artificial Sequence
  synthetic construct
NPQMQRS

SEQ ID NO: 123 - protein, 7 aa, Artificial Sequence
  synthetic construct
NPQMQRS

SEQ ID NO: 124 - protein, 7 aa, Artificial Sequence
  synthetic construct
NPQMQRS

SEQ ID NO: 125 - protein, 7 aa, Artificial Sequence
  synthetic construct
NYPTLKS

SEQ ID NO: 126 - protein, 7 aa, Artificial Sequence
  synthetic construct
NYPTLKS

SEQ ID NO: 127 - protein, 7 aa, Artificial Sequence
  synthetic construct
QNPRQIY

SEQ ID NO: 128 - protein, 7 aa, Artificial Sequence
  synthetic construct
QNPRQIY

SEQ ID NO: 129 - protein, 7 aa, Artificial Sequence
  synthetic construct
QNPRQIY

SEQ ID NO: 130 - protein, 7 aa, Artificial Sequence
  synthetic construct
QNPRQIY

SEQ ID NO: 131 - protein, 7 aa, Artificial Sequence
  synthetic construct
ETYARPL

SEQ ID NO: 132 - protein, 7 aa, Artificial Sequence
  synthetic construct
ETVCASS

SEQ ID NO: 133 - protein, 7 aa, Artificial Sequence
  synthetic construct
KPMQFVH

SEQ ID NO: 134 - protein, 7 aa, Artificial Sequence
  synthetic construct
KPMQFVH

SEQ ID NO: 135 - protein, 7 aa, Artificial Sequence
  synthetic construct
PAKQKAH

SEQ ID NO: 136 - protein, 7 aa, Artificial Sequence
  synthetic construct
PTTWGHL

SEQ ID NO: 137 - protein, 7 aa, Artificial Sequence
  synthetic construct
PTTWGHL

SEQ ID NO: 138 - protein, 7 aa, Artificial Sequence
  synthetic construct
SASGTPS

SEQ ID NO: 139 - protein, 7 aa, Artificial Sequence
  synthetic construct
SSYEYHA

SEQ ID NO: 140 - protein, 7 aa, Artificial Sequence
  synthetic construct
SSYEYHA

SEQ ID NO: 141 - protein, 7 aa, Artificial Sequence
  synthetic construct
STQAHPW

SEQ ID NO: 142 - protein, 7 aa, Artificial Sequence
  synthetic construct
TVLGTFP

SEQ ID NO: 143 - protein, 7 aa, Artificial Sequence
  synthetic construct
WYPNHLA

SEQ ID NO: 144 - protein, 7 aa, Artificial Sequence
  synthetic construct
TTYNSPP

SEQ ID NO: 145 - protein, 7 aa, Artificial Sequence
  synthetic construct
MTSQTLR

SEQ ID NO: 146 - protein, 12 aa, Artificial Sequence
  synthetic construct
WPANKLSTKSMY

SEQ ID NO: 147 - protein, 12 aa, Artificial Sequence
  synthetic construct
WPANKLSTKSMY

SEQ ID NO: 148 - protein, 12 aa, Artificial Sequence
  synthetic construct
NPYHPTIPQSVH

SEQ ID NO: 149 - protein, 7 aa, Artificial Sequence
  synthetic construct
DKLHRLA

SEQ ID NO: 150 - protein, 7 aa, Artificial Sequence
  synthetic construct
QPGLWPS

SEQ ID NO: 151 - protein, 7 aa, Artificial Sequence
  synthetic construct
ESLKSIS

SEQ ID NO: 152 - protein, 7 aa, Artificial Sequence
  synthetic construct
GSCPPKK

SEQ ID NO: 153 - protein, 7 aa, Artificial Sequence
  synthetic construct
GSLFKAL

SEQ ID NO: 154 - protein, 7 aa, Artificial Sequence
  synthetic construct
HQWDHKY

SEQ ID NO: 155 - protein, 7 aa, Artificial Sequence
  synthetic construct
LSAPMEY

SEQ ID NO: 156 - protein, 7 aa, Artificial Sequence
  synthetic construct
MKVHERS

SEQ ID NO: 157 - protein, 7 aa, Artificial Sequence
  synthetic construct
FVNLLGQ

SEQ ID NO: 158 - protein, 7 aa, Artificial Sequence
  synthetic construct
PIDAFFD

SEQ ID NO: 159 - protein, 7 aa, Artificial Sequence
  synthetic construct
PPNMARA

SEQ ID NO: 160 - protein, 7 aa, Artificial Sequence
  synthetic construct
PTNKPHT

SEQ ID NO: 161 - protein, 7 aa, Artificial Sequence
  synthetic construct
SPNNTRE

SEQ ID NO: 162 - protein, 7 aa, Artificial Sequence
  synthetic construct
SPEMKPR

SEQ ID NO: 163 - protein, 7 aa, Artificial Sequence
  synthetic construct
SSSMAKM

SEQ ID NO: 164 - protein, 7 aa, Artificial Sequence
  synthetic construct
TDHPPKA

SEQ ID NO: 165 - protein, 7 aa, Artificial Sequence
  synthetic construct
TLAFQTA

SEQ ID NO: 166 - protein, 7 aa, Artificial Sequence
  synthetic construct
APLSLSL

SEQ ID NO: 167 - protein, 7 aa, Artificial Sequence
  synthetic construct
HYPTVNF

SEQ ID NO: 168 - protein, 12 aa, Artificial Sequence
  synthetic construct
QHNFRGASSSAP

SEQ ID NO: 169 - protein, 12 aa, Artificial Sequence
  synthetic construct
  MISC_FEATURE (5)..(5): Xaa is Arg or Pro
HQFPXSNLVWKP

SEQ ID NO: 170 - protein, 12 aa, Artificial Sequence
  synthetic construct
LSLRASAATDFQ

SEQ ID NO: 171 - protein, 12 aa, Artificial Sequence
  synthetic construct
MQFTPAPSPSDH

SEQ ID NO: 172 - protein, 12 aa, Artificial Sequence
  synthetic construct
SVFLPTRHSPDL

SEQ ID NO: 173 - protein, 12 aa, Artificial Sequence
  synthetic construct
SVSVGMKPSPRP

SEQ ID NO: 174 - protein, 12 aa, Artificial Sequence
  synthetic construct
SVSVGMKPSPRP

SEQ ID NO: 175 - protein, 12 aa, Artificial Sequence
  synthetic construct
SVSVGMKPSPRP

SEQ ID NO: 176 - protein, 12 aa, Artificial Sequence
  synthetic construct
SVSVGMKPSPRP

SEQ ID NO: 177 - protein, 11 aa, Artificial Sequence
  synthetic construct
SVSVGMNAESA

SEQ ID NO: 178 - protein, 7 aa, Artificial Sequence
  synthetic construct
RHTLPLH

SEQ ID NO: 179 - protein, 7 aa, Artificial Sequence
  synthetic construct
NPQMQRS

SEQ ID NO: 180 - protein, 7 aa, Artificial Sequence
  synthetic construct
NYPTLKS

SEQ ID NO: 181 - protein, 7 aa, Artificial Sequence
  synthetic construct
NYPTLKS

SEQ ID NO: 182 - protein, 7 aa, Artificial Sequence
  synthetic construct
DMRQQRS

SEQ ID NO: 183 - protein, 7 aa, Artificial Sequence
  synthetic construct
QNPRQIY

SEQ ID NO: 184 - protein, 7 aa, Artificial Sequence
  synthetic construct
QNPRQIY

SEQ ID NO: 185 - protein, 7 aa, Artificial Sequence
  synthetic construct
QNPRQIY

SEQ ID NO: 186 - protein, 7 aa, Artificial Sequence
  synthetic construct
QNPRQIY

SEQ ID NO: 187 - protein, 7 aa, Artificial Sequence
  synthetic construct
QNPRQIY

SEQ ID NO: 188 - protein, 7 aa, Artificial Sequence
  synthetic construct
QNPRQIY

SEQ ID NO: 189 - protein, 7 aa, Artificial Sequence
  synthetic construct
QNPRQIY

SEQ ID NO: 190 - protein, 7 aa, Artificial Sequence
  synthetic construct
QNPRQIY

SEQ ID NO: 191 - protein, 7 aa, Artificial Sequence
  synthetic construct
QNPRQIY

SEQ ID NO: 192 - protein, 7 aa, Artificial Sequence
  synthetic construct
QTHSSLW

SEQ ID NO: 193 - protein, 7 aa, Artificial Sequence
  synthetic construct
ETYQQPL

SEQ ID NO: 194 - protein, 7 aa, Artificial Sequence
  synthetic construct
ETYARPL

SEQ ID NO: 195 - protein, 7 aa, Artificial Sequence
  synthetic construct
GTSRLFS

SEQ ID NO: 196 - protein, 7 aa, Artificial Sequence
  synthetic construct
LTQTLQY

SEQ ID NO: 197 - protein, 7 aa, Artificial Sequence
  synthetic construct
KAFDKHG

SEQ ID NO: 198 - protein, 7 aa, Artificial Sequence
  synthetic construct
RPMQFVH

SEQ ID NO: 199 - protein, 7 aa, Artificial Sequence
  synthetic construct
KPMQFVH

SEQ ID NO: 200 - protein, 7 aa, Artificial Sequence
  synthetic construct
KPMQFIH

SEQ ID NO: 201 - protein, 7 aa, Artificial Sequence
  synthetic construct
PAKQKAH

SEQ ID NO: 202 - protein, 7 aa, Artificial Sequence
  synthetic construct
SASGTPS

SEQ ID NO: 203 - protein, 7 aa, Artificial Sequence
  synthetic construct
SSHHHRH

SEQ ID NO: 204 - protein, 7 aa, Artificial Sequence
  synthetic construct
SSYEYHA

SEQ ID NO: 205 - protein, 7 aa, Artificial Sequence
  synthetic construct
TGPTSLS

SEQ ID NO: 206 - protein, 12 aa, Artificial Sequence
  synthetic construct
LRAFPSLPHTVT

SEQ ID NO: 207 - protein, 7 aa, Artificial Sequence
  synthetic construct
NPRSQAT

SEQ ID NO: 208 - protein, 7 aa, Artificial Sequence
  synthetic construct
HRLGHMS

SEQ ID NO: 209 - protein, 7 aa, Artificial Sequence
  synthetic construct
LLPLKFK

SEQ ID NO: 210 - protein, 7 aa, Artificial Sequence
  synthetic construct
LPSIHNL

SEQ ID NO: 211 - protein, 7 aa, Artificial Sequence
  synthetic construct
KATITGM

SEQ ID NO: 212 - protein, 7 aa, Artificial Sequence
  synthetic construct
PDIPLSR

SEQ ID NO: 213 - protein, 7 aa, Artificial Sequence
  synthetic construct
PSMKHWR

SEQ ID NO: 214 - protein, 7 aa, Artificial Sequence
  synthetic construct
SAKGRAD

SEQ ID NO: 215 - protein, 7 aa, Artificial Sequence
  synthetic construct
SRTGAHH

SEQ ID NO: 216 - protein, 7 aa, Artificial Sequence
  synthetic construct
SKTSSTS

SEQ ID NO: 217 - protein, 7 aa, Artificial Sequence
  synthetic construct
SPNNPRE

SEQ ID NO: 218 - protein, 7 aa, Artificial Sequence
  synthetic construct
TLQRMGQ

SEQ ID NO: 219 - protein, 7 aa, Artificial Sequence
  synthetic construct
TMTNMAK

SEQ ID NO: 220 - protein, 7 aa, Artificial Sequence
  synthetic construct
TTLSPRT

SEQ ID NO: 221 - protein, 7 aa, Artificial Sequence
  synthetic construct
TTKNFNK

SEQ ID NO: 222 - protein, 7 aa, Artificial Sequence
  synthetic construct
YPKALRN

SEQ ID NO: 223 - protein, 7 aa, Artificial Sequence
  synthetic construct
VVKSNGE

SEQ ID NO: 224 - protein, 5 aa, Artificial Sequence
  synthetic construct
ITGAY

SEQ ID NO: 225 - protein, 7 aa, Artificial Sequence
  synthetic construct
LPLTPLP

SEQ ID NO: 226 - protein, 12 aa, Artificial Sequence
  synthetic construct
HSMPHMGTYLLT

SEQ ID NO: 227 - protein, 12 aa, Artificial Sequence
  synthetic construct
MQFTPAPSPSDH

SEQ ID NO: 228 - protein, 12 aa, Artificial Sequence
  synthetic construct
MPQTLVLPRSLL

SEQ ID NO: 229 - protein, 12 aa, Artificial Sequence
  synthetic construct
SSTQVQHTLLQT

SEQ ID NO: 230 - protein, 12 aa, Artificial Sequence
  synthetic construct
SWPLYSRDSGLG

SEQ ID NO: 231 - protein, 12 aa, Artificial Sequence
  synthetic construct
  MISC_FEATURE (11)..(11): Xaa is any amino acid
SVSVGTEAESXA

SEQ ID NO: 232 - protein, 12 aa, Artificial Sequence
  Synthetic Construct
SVSVGMKPSPRP

SEQ ID NO: 233 - protein, 12 aa, Artificial Sequence
  Synthetic Construct
SVSVGMKPSPRP

SEQ ID NO: 234 - protein, 12 aa, Artificial Sequence
  Synthetic Construct
SVSVGMKPSPRP

SEQ ID NO: 235 - protein, 12 aa, Artificial Sequence
  Synthetic Construct
SVSVGMNAESYG

SEQ ID NO: 236 - protein, 12 aa, Artificial Sequence
  Synthetic Construct
THPVVFEDERLF

SEQ ID NO: 237 - protein, 12 aa, Artificial Sequence
  Synthetic Construct
TLPSPLALLTVH

SEQ ID NO: 238 - protein, 12 aa, Artificial Sequence
  Synthetic Construct
WPTYLNPSSLKA

SEQ ID NO: 239 - protein, 7 aa, Artificial Sequence
  Synthetic Construct
ASHNPKL

SEQ ID NO: 240 - protein, 7 aa, Artificial Sequence
  Synthetic Construct
PAKQKAH

SEQ ID NO: 241 - protein, 7 aa, Artificial Sequence
  Synthetic Construct
PAKQKAH

SEQ ID NO: 242 - protein, 7 aa, Artificial Sequence
  Synthetic Construct
SASGTPS

SEQ ID NO: 243 - protein, 7 aa, Artificial Sequence
  Synthetic Construct
TRFYDSL

SEQ ID NO: 244 - protein, 7 aa, Artificial Sequence
  Synthetic Construct
QNPRQIY

SEQ ID NO: 245 - protein, 7 aa, Artificial Sequence
  Synthetic Construct
QNPRQIY

SEQ ID NO: 246 - protein, 7 aa, Artificial Sequence
  Synthetic Construct
QNPRQIY

SEQ ID NO: 247 - protein, 7 aa, Artificial Sequence
  Synthetic Construct
QNPRQIY

SEQ ID NO: 248 - protein, 7 aa, Artificial Sequence
  Synthetic Construct
TGPTSLS

SEQ ID NO: 249 - protein, 7 aa, Artificial Sequence
  Synthetic Construct
TGPTSLS

SEQ ID NO: 250 - protein, 7 aa, Artificial Sequence
  Synthetic Construct
NPQMQRS

SEQ ID NO: 251 - protein, 7 aa, Artificial Sequence
  Synthetic Construct
NPQMQRS

SEQ ID NO: 252 - protein, 7 aa, Artificial Sequence
  Synthetic Construct
NPQMQRS

SEQ ID NO: 253 - protein, 7 aa, Artificial Sequence
  Synthetic Construct
NPQMQRS

SEQ ID NO: 254 - protein, 7 aa, Artificial Sequence
  Synthetic Construct
NPQMQRS

SEQ ID NO: 255 - protein, 7 aa, Artificial Sequence
  Synthetic Construct
KPMQFVH

SEQ ID NO: 256 - protein, 7 aa, Artificial Sequence
  Synthetic Construct
SSYEYHA

SEQ ID NO: 257 - protein, 7 aa, Artificial Sequence
  Synthetic Construct
STQAHPW

SEQ ID NO: 258 - protein, 7 aa, Artificial Sequence
  Synthetic Construct
GTSRLFS

SEQ ID NO: 259 - protein, 7 aa, Artificial Sequence
  Synthetic Construct
NYPTLKS

SEQ ID NO: 260 - protein, 7 aa, Artificial Sequence
  Synthetic Construct
NYPTLKS

SEQ ID NO: 261 - protein, 7 aa, Artificial Sequence
  Synthetic Construct
NYPTLKS

SEQ ID NO: 262 - protein, 7 aa, Artificial Sequence
  Synthetic Construct
NYPTLKS

SEQ ID NO: 263 - protein, 7 aa, Artificial Sequence
  Synthetic Construct
NYPTLKS

SEQ ID NO: 264 - protein, 7 aa, Artificial Sequence
  Synthetic Construct
NYPTLKS

SEQ ID NO: 265 - protein, 7 aa, Artificial Sequence
  Synthetic Construct
NYPTLKS

SEQ ID NO: 266 - protein, 7 aa, Artificial Sequence
  Synthetic Construct
HAPVQPN

SEQ ID NO: 267 - protein, 12 aa, Artificial Sequence
  Synthetic Construct
NPYHPTIPQSVH

SEQ ID NO: 268 - protein, 12 aa, Artificial Sequence
  Synthetic Construct
NPYHPTIPQSVH

SEQ ID NO: 269 - protein, 12 aa, Artificial Sequence
  Synthetic Construct
NPYHPTIPQSVH

SEQ ID NO: 270 - protein, 12 aa, Artificial Sequence
  Synthetic Construct
NPYHPTIPQSVH

SEQ ID NO: 271 - protein, 12 aa, Artificial Sequence
  Synthetic Construct
HQFISPEPFLIS

SEQ ID NO: 272 - protein, 17 aa, Artificial Sequence
  Synthetic Construct
SPNFSWLPLGTTSPNFS

SEQ ID NO: 273 - protein, 7 aa, Artificial Sequence
  Synthetic Construct
WLPLGTT

SEQ ID NO: 274 - protein, 12 aa, Artificial Sequence
  Synthetic Construct
SVSVGMKPSPRP

SEQ ID NO: 275 - protein, 12 aa, Artificial Sequence
  Synthetic Construct
SVSVGMKPSPRP

SEQ ID NO: 276 - protein, 12 aa, Artificial Sequence
  Synthetic Construct
TPLTSPSLVRPQ

SEQ ID NO: 277 - protein, 12 aa, Artificial Sequence
  Synthetic Construct
TPLSYLKGLVTV

SEQ ID NO: 278 - protein, 7 aa, Artificial Sequence
  Synthetic Construct
NPMIMNQ

SEQ ID NO: 279 - protein, 7 aa, Artificial Sequence
  Synthetic Construct
NPMIMNQ

SEQ ID NO: 280 - protein, 7 aa, Artificial Sequence
  Synthetic Construct
NITQLGS

SEQ ID NO: 281 - protein, 7 aa, Artificial Sequence
  Synthetic Construct
HTLLSTT

SEQ ID NO: 282 - protein, 7 aa, Artificial Sequence
  Synthetic Construct
HTLLSTT

SEQ ID NO: 283 - protein, 7 aa, Artificial Sequence
  Synthetic Construct
HTLLSTT

SEQ ID NO: 284 - protein, 7 aa, Artificial Sequence
  Synthetic Construct
LGPGKAF

SEQ ID NO: 285 - protein, 7 aa, Artificial Sequence
  Synthetic Construct
LGPGKAF

SEQ ID NO: 286 - protein, 7 aa, Artificial Sequence
  Synthetic Construct
LGPGKAF

SEQ ID NO: 287 - protein, 7 aa, Artificial Sequence
  Synthetic Construct
LGPGKAF

SEQ ID NO: 288 - protein, 7 aa, Artificial Sequence
  Synthetic Construct
KTSSWAN

SEQ ID NO: 289 - protein, 7 aa, Artificial Sequence
  Synthetic Construct
KMNHMPN

SEQ ID NO: 290 - protein, 7 aa, Artificial Sequence
  Synthetic Construct
SLLTPWL

SEQ ID NO: 291 - protein, 7 aa, Artificial Sequence
  Synthetic Construct
TLGLPML

SEQ ID NO: 292 - protein, 6 aa, Artificial Sequence
  Synthetic Construct
TGLAKT

SEQ ID NO: 293 - protein, 5 aa, Artificial Sequence
  Synthetic Construct
IRLIS

SEQ ID NO: 294 - protein, 7 aa, Artificial Sequence
  Synthetic Construct
LGPGKAF

SEQ ID NO: 295 - protein, 7 aa, Artificial Sequence
  Synthetic Construct
LGPGKAF

SEQ ID NO: 296 - protein, 7 aa, Artificial Sequence
  Synthetic Construct
LGPGKAF

SEQ ID NO: 297 - protein, 12 aa, Artificial Sequence
  Synthetic Construct
DLNYFTLSSKRE

SEQ ID NO: 298 - protein, 12 aa, Artificial Sequence
  Synthetic Construct
DLNYFTLSSKRE

SEQ ID NO: 299 - protein, 12 aa, Artificial Sequence
  Synthetic Construct
TMGFTAPRFPHY

SEQ ID NO: 300 - protein, 12 aa, Artificial Sequence
  Synthetic Construct
TMGFTAPRFPHY

SEQ ID NO: 301 - protein, 12 aa, Artificial Sequence
  Synthetic Construct
TMGFTAPRFPHY

SEQ ID NO: 302 - protein, 12 aa, Artificial Sequence
  Synthetic Construct
TMGFTAPRFPHY

SEQ ID NO: 303 - protein, 7 aa, Artificial Sequence
  Synthetic Construct
HTLLSTT

SEQ ID NO: 304 - protein, 7 aa, Artificial Sequence
  Synthetic Construct
HTLLSTT

SEQ ID NO: 305 - protein, 7 aa, Artificial Sequence
  Synthetic Construct
LASTTHV

SEQ ID NO: 306 - protein, 7 aa, Artificial Sequence
  Synthetic Construct
LGPGKAF

SEQ ID NO: 307 - protein, 7 aa, Artificial Sequence
  Synthetic Construct
LGPGKAF

SEQ ID NO: 308 - protein, 7 aa, Artificial Sequence
  Synthetic Construct
LGPGKAF

SEQ ID NO: 309 - protein, 7 aa, Artificial Sequence
  Synthetic Construct
SLLTPWL

SEQ ID NO: 310 - protein, 7 aa, Artificial Sequence
  Synthetic Construct
NERQMEL

SEQ ID NO: 311 - protein, 7 aa, Artificial Sequence
  Synthetic Construct
NKPLSTL

SEQ ID NO: 312 - protein, 7 aa, Artificial Sequence
  Synthetic Construct
HTLLSTT

SEQ ID NO: 313 - protein, 7 aa, Artificial Sequence
  Synthetic Construct
LKPFSGA

SEQ ID NO: 314 - protein, 7 aa, Artificial Sequence
  Synthetic Construct
LGPGKAF

SEQ ID NO: 315 - protein, 7 aa, Artificial Sequence
  Synthetic Construct
LGPGKAF

SEQ ID NO: 316 - protein, 7 aa, Artificial Sequence
  Synthetic Construct
LGPGKAF

SEQ ID NO: 317 - protein, 7 aa, Artificial Sequence
  Synthetic Construct
LGPGKAF

SEQ ID NO: 318 - protein, 7 aa, Artificial Sequence
  Synthetic Construct
STSAKHW

SEQ ID NO: 319 - protein, 12 aa, Artificial Sequence
  Synthetic Construct
TMGFTAPRFPHY

SEQ ID NO: 320 - protein, 12 aa, Artificial Sequence
  Synthetic Construct
TMGFTAPRFPHY

SEQ ID NO: 321 - protein, 12 aa, Artificial Sequence
  Synthetic Construct
TMGFTAPRFPHY

SEQ ID NO: 322 - protein, 12 aa, Artificial Sequence
  Synthetic Construct
TMGFTAPRFPHY

SEQ ID NO: 323 - protein, 12 aa, Artificial Sequence
  Synthetic Construct
TMGFTAPRFPHY

SEQ ID NO: 324 - protein, 12 aa, Artificial Sequence
  Synthetic Construct
TMGFTAPRFPHY

SEQ ID NO: 325 - protein, 12 aa, Artificial Sequence
  Synthetic Construct
TMGFTAPRFPHY

SEQ ID NO: 326 - protein, 9 aa, Artificial Sequence
  Synthetic Construct
CNYPTLKSC

SEQ ID NO: 327 - protein, 5 aa, Artificial Sequence
  Synthetic Construct
SNVIS

SEQ ID NO: 328 - protein, 17 aa, Artificial Sequence
  Synthetic Construct
GVIPIVDIANYAQRFKG

SEQ ID NO: 329 - protein, 11 aa, Artificial Sequence
  Synthetic Construct
TLGLVLDAMDY

SEQ ID NO: 330 - protein, 12 aa, Artificial Sequence
  Synthetic Construct
RASQSLGSSYLA

SEQ ID NO: 331 - protein, 7 aa, Artificial Sequence
  Synthetic Construct
GASSRAP

SEQ ID NO: 332 - protein, 9 aa, Artificial Sequence
  Synthetic Construct
QQYADSPIT

SEQ ID NO: 333 - protein, 120 aa, Artificial Sequence
  Synthetic Construct
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSS

SEQ ID NO: 334 - protein, 108 aa, Artificial Sequence
  Synthetic Construct
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 335 - protein, 3 aa, Artificial Sequence
  Synthetic Construct
GGG

SEQ ID NO: 336 - protein, 5 aa, Artificial Sequence
  Synthetic Construct
GGGGS

SEQ ID NO: 337 - protein, 16 aa, Artificial Sequence
  Synthetic Construct
GGGGSGGGGSGGGGSG